(19) United States
(12) Patent Application Publication
(10) Pub. No.: US 2025/0368645 A1
(43) Pub. Date: Dec. 4, 2025

(54) BETA CARBOLINE ANALOGUES AS SELECTIVE AND BIASED KAPPA OPIOID RECEPTORS AGONISTS FOR TREATING VARIOUS ASSOCIATED PATHOPHYSIOLOGICAL CONDITIONS

(71) Applicant: COUNCIL OF SCIENTIFIC AND INDUSTRIAL RESEARCH, New Dehli (IN)

(72) Inventors: Sanjay BATRA, Lucknow (IN); Prem Narayan YADAV, Lucknow (IN); Veena YADAV, Lucknow (IN); Lalan KUMAR, Lucknow (IN); Shalini DOGRA, Lucknow (IN); Poonam KUMARI, Lucknow (IN); Ajeet KUMAR, Lucknow (IN); Adilakshmi VUTLA, Lucknow (IN)

(21) Appl. No.: 18/876,855

(22) PCT Filed: Jun. 24, 2023
(86) PCT No.: PCT/IN2023/050611

§ 371 Date: Dec. 19, 2024

(30) Foreign Application Priority Data
Jun. 24, 2022 (IN) 202211036633

(51) Int. Cl. C07D 471/14; A61K 31/437; A61K 31/475; A61P 25/02; A61P 25/04

(52) U.S. Cl. CPC C07D 471/14; A61K 31/437; A61K 31/475; A61P 25/02; A61P 25/04

(57) ABSTRACT

The present invention relates selective for Kappa opioid receptor (KOR) agonist compounds, their tautomeric forms, their pharmaceutically accepted salts, or prodrugs thereof, which are useful for the treatment or prevention of various diseases in which the KOR are implicated, such as treatment of prevention of neuropathic pain, visceral pain, drug addiction, hyperalgesia, arthritic inflammation or autoimmune inflammation. The invention also relates to a process for the manufacture of said compounds and pharmaceutical compositions containing them and their use.

### FIELD OF INVENTION

The present invention relates to selective Kappa opioid receptor (KOR) agonist compounds, their tautomeric forms, their pharmaceutically accepted salts and prodrugs thereof, which are useful in the treatment or prevention of various diseases in which the KOR is implicated. The invention also relates to the process for preparing the said compounds and the pharmaceutical compositions containing them and their use in treatment of prevention of neuropathic pain, visceral pain, drug addiction, hyperalgesia, arthritic inflammation or autoimmune inflammation.

### BACKGROUND OF THE INVENTION

G protein-coupled receptors (GPCRs) represent the largest family of receptors on the cell surface in eukaryotes. The opioid receptors belong to this superfamily of GPCRs and are the molecular target of extensively prescribed analgesics such as morphine. Similar to other opioids, morphine produces adverse effects, including euphoria, respiratory depression, nausea. Repeated administration of most opioids leads to the development of tolerance and physical dependence, the most prominent challenge in effective clinical usage of opioids. The specific biological targets of opioids were first detected in the early 1970s by using radioactive ligand binding assays (Pert, C. B.; Pasternak, G.; Snyder, S. H. Science. 1973, 182, 1359-1361; Simon, E. J.; Hiller, J. M.; Edelman, I. Proc. Nat. Acad. Sci. 1973, 70, 1947-1949). Based on the binding profiles with the specific types of ligands, opioid receptors are categorized into three distinct receptors: μ opioid receptor (MOR), δ opioid receptor (DOR), and κ opioid receptor (KOR). In contrast to MOR and DOR, stimulation of KOR by its endogenous ligand dynorphin-A or synthetic agonists inhibits dopamine release in various brain areas. Besides induction of analgesia, several preclinical studies have shown that activation of KOR induces an aversive state and thereby counter the rewarding and addictive effects of nicotine, alcohol and cocaine (Bruchas, M. R.; Land, B. B.; Chavkin, C. Brain Res. 2010, 1314, 44-55; Knoll, A. T.; Carlezon Jr, W. A. Brain Res. 2010, 1314, 56-73). However, KOR agonists have also been shown to produce aversive mood disorders and facilitate drug relapse (Land, B. B.; Bruchas, M. R.; Schattauera, S.; Giardino, W. J.; Aita, M.; Messinger, D.; Hnasko, T. S.; Palmiter, R. D.; Chavkin, C. PNAS. 2009, 106, 19168-19173). These contradictory two-fold actions of KOR agonists allude to the complex physiological role of KOR and underline the unmet need for diverse KOR selective chemical probes to facilitate further research in this direction.

Compounds, which exhibit full agonist activity at the KOR have been widely shown to be efficacious in the preclinical models of pain, particularly visceral pain. KOR agonists are understood to lack several of the side effects of MOR agonists, including abuse liability, gastrointestinal transit inhibition and respiratory depression. However, they are unknown to produce complicating side effects, such as dysphoria and sedation at analgesic doses, which limit the development of KOR agonists as clinically useful analgesics.

Beyond analgesia, KOR agonists have shown anti-inflammatory effects both in vitro and in vivo (Soeberdt, M.; Molenveld, P.; Storcken, R. P. M.; Mazery, R. B. D.; Sterk, G. J.; Autar, R.; Bolster, M. G.; Wagner, C.; Aerts, S. N. H.; Holst, F. R. V.; Wegert, A.; Tangherlini, G.; Frehland, B.; Schepmann, D.; Metze, D.; Lotts, T.; Knie, U.; Lin, K. U.; Huang, T. Y.; Lai, C. C.; Stander, S.; Wiinsch, B.; Abels, C. J. Med. Chem. 2017, 60, 2526-2551; Parkhill, A. L.; Bidlack. J. M. Int. Immunopharmacol. 2006, 6, 1013-1019). Additionally, asimadoline, a peripheral kappa opioid agonist (moderately restricted to the periphery due to lack of blood-brain barrier penetration), is currently in a phase-2 clinical trial for the treatment of pruritus and being researched for irritable bowel syndrome (Joshi, S. K.; Su, X.; Porreca, F.; Gebhart, G. F. J. Neurosci. 2000, 20, 5874-5829). Due to its limited CNS entry, asimadoline was thought to have reduced adverse effects related to CNS-specific KOR activation such as dysphoria and anhedonia may reduce the extent of side-effects associated with mood. Additional known kappa opioid agonists, such as enadoline and spiradoline, enter the central nervous system (CNS) causing dysphoria, and thus have not been developed clinically. Further, while mixed agonists (acting at MOR and KOR) have been marketed, to date, no full KOR agonist has been approved for use in humans.

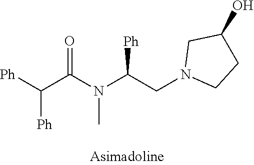 [SMILES: CN(C(=O)C(c1ccccc1)c1ccccc1)[C@H](CN1CC[C@H](O)C1)c1ccccc1]

Given that GPC mediates multiple signalling pathways in the cell, agonists that show higher potency to specific signalling pathways over others are known as “biased agonists” and have been shown to exhibit better therapeutic index (Walters, R. W.; Whalen, E. J.; Lefkowitz, R. J. J. Clin. Invest. 2009, 119, 1312-1321). This new and rapidly evolving concept has revitalized interest for many GPCR targets. To support this notion, KOR selective G-protein biased agonist 22-thiocyanatosalvinorin A (RB-64) has been shown to induce analgesia and aversion, and unlike unbiased KOR agonists (such as U69593) it does not induce sedation and anhedonia. These data indicate that a mechanism other than activation of G protein signalling might mediate these adverse effects (White, K. L.; Robinson, J. E.; Zhu, H.; DiBerto, J. F.; Polepally, P. R. Zjawiony, J. K.; Nichols, D. E.; Malanga, C. J.; Roth, B. L. J Pharmacol Exp Ther. 2015, 352, 98-109). However, this study lacks the data on RB64 availability in the brain, which limits the interpretation of the lack of CNS-specific adverse effects.

Thus, there is a need for a KOR agonist that retains sufficient efficacy to treat various types of pain and other symptoms or disease states involving KOR, and simultaneously reduce the CNS-associated side effects. The present invention seeks to address these and other needs.

Crystal structure of human KOR in complex with MP1104, a potent dual agonist of DOR and KOR, and an active-state-stabilizing nanobody revealed substantial conformational changes in the binding pocket and intracellular and extracellular regions (Che, T.; Majumdar, S.; Zaidi, S. A.; Katritch, V.; Wacker, D.; Roth, B. L. Cell, 2018, 172,55-67). Although MP1 104 exhibited robust analgesic efficacy in a rodent model without the adverse effect of conditioned place aversion or preference, it is not known if it has biased agonist property (Varadi, A.; Marrone, G. F.; Eans, S. O.; Ganno, M. L.; Subrath, J. J.; Rouzic, V. L.; Hunkele, A.; Pasternak, G. W.; McLaughlin, J. P. ACS Chem. Neurosci. 2015, 6, 1813-1824). Arylacetamide derivatives are considered to be the most important selective KOR agonists developed so far (Szmuszkovicz, J.; Voigtlander, P. F. V. J. Med. Chem. 1982, 25, 1125-1126; Hunter, J. C.; Leighton, G. E.; Meecham, K. G.; Boyle, S. J.; Horwell, D. C.; Rees, D. C.; Hughes, J. Br. J. Pharmacol. 1990, 101, 183-189; Emmerson, p. j.; Liu, M. R.; Woods, J. H.; Medzihradsky, F. J Pharmacol Exp Ther. 1994, 271, 1630-1637). After the discovery of the first selective arylacetamide κ-agonists U-50,488, in the early 1970s, which displayed analgesic effects in vivo without producing respiratory depression, constipation, or tolerance, several new diverse arylacetamide κ-agonists have been disclosed (Lahti, R. A.; VonVoigtlander, P. F.; Barsuhn. C. Life Sci. 1982, 31, 2257-2260; Lathi, R. A.; Mickelson, M. M.; Mccall, J. M.; Voigtlander, P. F. V. Eur. J. Pharmacol, 1985, 109, 281-284; Rajagopalan, K. V.; Johnson, J. L. J. Biol. Chem, 1992, 267, 10199-10202). Among the disclosed compounds, ICI 199441, was 146-fold more potent than U-50,488 in the in vitro assay. Unfortunately, these centrally acting κ-agonists produced their own set of CNS side-effects such as dysphoria and diuresis, which prevented their further development as analgesics. Nevertheless, there is a growing interest to develop peripherally acting KOR agonists that have limited or no access to the CNS in an effort to reduce or eliminate their side effects (Stein, C.; Lang, J. L. Curr Opin Pharmacol. 2009, 9, 3-8).

Several modifications such as the introduction of a charged group into ligands were attempted for enhancing their CNS/peripheral nervous system selectivity. This underscores the need for identifying selective and potent opioid ligands with high KOR-agonist activity and low CNS penetration (DeHaven-Hudkins. D. L.; Dolle, R. E. Curr. Pharm. Des. 2004, 10, 743-757).

U.S. Pat. No. 5,688,955 discloses substituted piperidines, substituted naphthalenes, aryl-substituted amides and cyclohexyl-substituted amides bearing the following general formula having KOR agonistic activity.

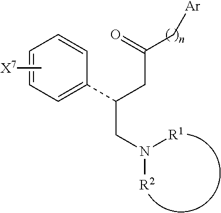

U.S. Pat. No. 5,804,595 discloses amino acid conjugates of substituted 2-phenyl-N-[1-(phenyl)-2-(1-heterocycloalkyl- or heteroccycloaryl)-ethyl]acetamides allegedly useful for selectively agonizing KOR in mammalian tissue.

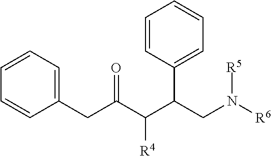

U.S. Pat. No. 6,133,307 discloses KOR agonists, which are useful in the treatment of arthritis, hypertension, pain, inflammation, migraine, inflammatory disorders of the gastrointestinal tract and psoriasis.

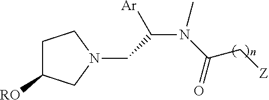

WO patent no 2015/114660 A1 disclose selective and peripherally acting KOR agonists which are useful in the treatment or prevention of diseases such as visceral pain, hyperalgesia, rheumatoid arthritis inflammation, osteoarthritic inflammation, otitic inflammation, IBS inflammation, ocular inflammation or autoimmune inflammation.

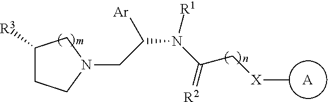

US patent number 20160235759 discloses a new series of potent and selective KOR agonist, which may offer an effective, better-tolerated treatment for chronic pain.

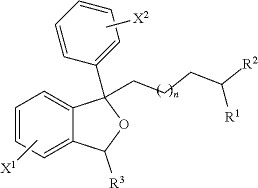

Cara Therapeutics recently announced their collaboration with the Japanese firm Maruishi to develop and commercialize CR845, a peripherally restricted KOR agonist for the treatment of chronic pain. This compound did not display dysphoric behaviour and hallucination in Phase II clinical trials in the US, it also showed a reduced incidence of nausea and vomiting in post-operative patient. Nektar Therapeutics of San Francisco, Calif. has also presented the pre-clinical data for their http://ir.nektar.com/releasedetail.cfm?ReleaseID=871723) oral peripherally acting KOR agonist NKTR-195.

Yadav et al., 2021 (Yadav. V. D.; Kumar, L.; Kumari. P.; Kumar, S.; Singh, M.; Siddiqi. M. I.; Yadav P. N.; Batra, S. Chem Med Chem 2021, 16, 1917-1926) reported new carboline derivatives, which were found to display significant biased KOR agonist activity that may be useful for treating pain.

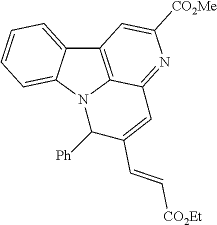 [SMILES: CCOC(=O)/C=C/C1=Cc2nc(C(=O)OC)cc3c4ccccc4n(c23)C1c1ccccc1]

Until date, only mixed agonists (acting at MOR and KOR) have been marketed and no biased KOA has been approved for use in humans. Therefore, there is the need to discover diverse biased KOR selective chemical probes (biased KOA) that retain sufficient efficacy to treat various types of pain and other symptoms or disease states associated with the KOR and simultaneously either have reduced or are devoid of the CNS associated side effects. The compounds of the present invention display potent biased KOR agonist activity and were found to be highly efficacious in alleviating hyperalgesia and allodynia and importantly did not induce sedation and neuromuscular incoordination. Consequently, these compounds may be advanced to clinics for treating pain.

### SUMMARY OF THE INVENTION

Accordingly, the present invention provides a compound of formula I,

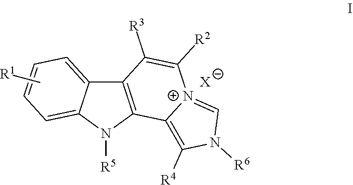

tautomer, stereoisomer or pharmaceutically acceptable salts thereof, having biased KOR agonist activity and are useful for the treatment of pain, substance abuse, and pruritus wherein:
   R¹ is selected from hydrogen, halogen, alkoxy, benzyloxy, sulphonic acid, sulphonamide, cyano, nitro or amide;
   R² is selected from hydrogen, C₁₋₂ alkyl, alkyloycarbonyl or aryloxycarbonyl;
   R³ is selected from hydrogen or aryl;
   R² and R³ may jointly form an aromatic ring having substituent selected from hydrogen, trifluoromethyl, nitro or halogen;
   R₄ is selected from substituted or unsubstituted alkyl, aryl or heteroaryl, wherein the substitutents are selected from the groups consisting of halogen, alkyl, aryl, alkoxy, aryloxy, thiomethyl, trifluoromethyl, trifluoromethyloxy, thioalkyl, sulphonylamide and nitro;
   R⁵ is selected from H, alkyl, alkenyl, cycloalkyl aryl, arylalkyl, heteroaryl, benzoyl, sulphonylphenyl, or alkoxycarbonyl;
   R⁶ is selected from substituted or unsubstituted alkyl, aryl, arylalkyl, 5 or 6-membered heteroaryl, alkoxycarbonylalkyl, cycloalkyl, or hetercycloalkyl, wherein the substitutents are selected from halogen, alkyl, alkoxy, aryl or alkyl aminoalkyl; and
   X is selected from Cl, I, ClO₄, CF₃SO₂, BF₄ or PF₆.

In an embodiment, the present invention provides a compound of formula I, wherein R¹, R², R³ and R⁵ are H; R⁴ is selected from aryl, heteroaryl optionally substituted with halogen, alkyl, alkoxy or nitro and R⁶ is selected from alkyl, aryl, arylalkyl, 5 or 6-membered heteroaryl optionally substituted with halogen, alkyl, alkoxy and aryl.

In an embodiment, the present invention provides a compound of formula I, wherein R¹ and R⁵ are H; R² and R³ jointly form an aromatic ring, R⁴ is selected from aryl, heteroaryl optionally substituted with halogen, alkyl, alkoxy or nitro and R⁶ is alkyl, aryl, arylalkyl, 5 or 6-membered heteroaryl optionally substituted with halogen, alkyl, alkoxy and aryl.

The present invention also provides a process for preparing the compound of formula I,

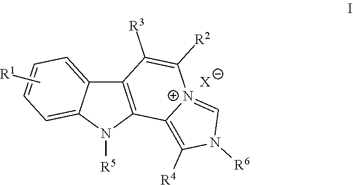

wherein:
R¹ is selected from hydrogen, halogen, alkoxy, benzyloxy, sulphonic acid, sulphonamide, cyano, nitro or amide;
R² is selected from hydrogen, C₁₋₂ alkyl, alkyloycarbonyl or aryloxycarbonyl;
R³ is selected from hydrogen or aryl;
R² and R³ may jointly form an aromatic ring having substituent selected from hydrogen, trifluoromethyl, nitro or halogen;
R⁴ is selected from substituted or unsubstituted alkyl, aryl or heteroaryl, wherein the substitutents are selected from the groups consisting of halogen, alkyl, aryl, alkoxy, aryloxy, thiomethyl, trifluoromethyl, trifluoromethyloxy, thioalkyl, sulphonylamide or nitro;
R⁵ is selected from H, alkyl, alkenyl, aryl, arylalkyl, heteroaryl, benzoyl, sulphonylphenyl, or alkoxycarbonyl;
R⁶ is selected from substituted or unsubstituted alkyl, aryl, arylalkyl, 5 or 6-membered heteroaryl, alkoxycarbonylalkyl, cycloalkyl, hetercycloalkyl, wherein the substitutents are selected from halogen, alkyl, alkoxy, aryl or alkyl aminoalkyl; and
X is selected from Cl, I, ClO₄, CF₃SO₂, BF₄ or PF₆.
the process comprising the steps of:
a) reacting an indole derivative of formula A with a compound of formula A′ selected from arylpyruvaldehyde, terminal alkyne or substituted acetophene in the presence of iodine in DMSO as solvent for 1 to 12 h at rt −110° C., washing the reaction mixture with Na₂S₂O₃ solution and extracting with an organic solvent to obtain a compound of formula B,

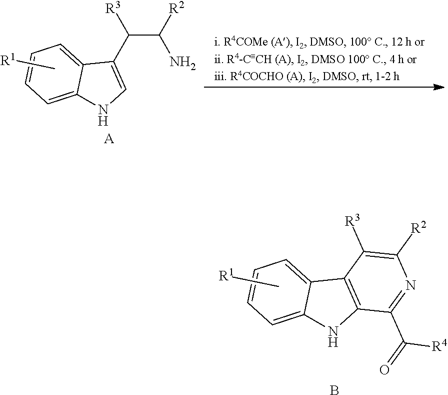

wherein R¹, R², R³ and R⁴ are as defined above;
b) evaporating the organic solvent and performing the column chromatography over silica gel using hexane:ethylacetate as eluent to purify the compound of formula B;
c) reacting the compound of formula B with R⁵X, wherein R⁵ is selected from H, alkyl, alkenyl, aryl, arylalkyl, heteroaryl, benzoyl, sulphonylphenyl, alkoxycarbonyl and X is a halogen, in the presence of cesium carbonate in DMF and adding crushed ice to obtain solid precipitate of the N-substituted carboline compound of formula C;

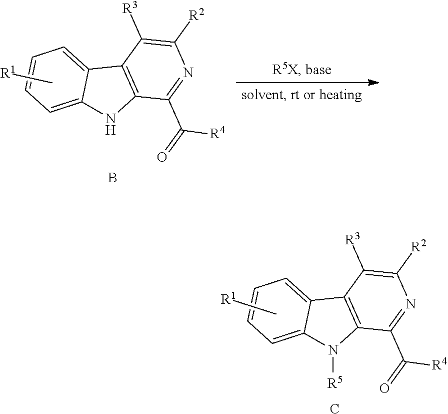

d) reacting formaldehyde with the substituted β-carboline compound of formula B or C and amine of formula R⁶NH₂, wherein R⁶ is selected from substituted or unsubstituted alkyl, aryl, arylalkyl, 5 or 6-membered heteroaryl, alkoxycarbonylalkyl, cycloalkyl, or hetercycloalkyl, wherein the substitutents are selected from halogen, alkyl, alkoxy, aryl or alkyl aminoalkyl, to obtain compound for formula I;

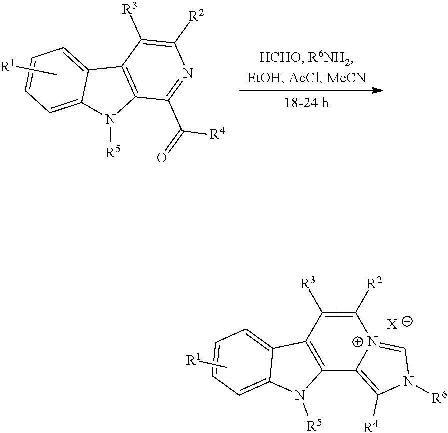

e) optionally purifying the compound for formula I by column chromatography over silica gel (MeOH/EtOAc).

In a preferred embodiment of the present invention the indole derivative used in the process is selected from the group consisting of tryptamine, alkyl tryptophan ester and 2-(1H-indole-3-yl)aniline.

In a preferred embodiment of the present invention the organic solvent is selected from ethyl acetate, dichloromethane or chloroform.

In a preferred embodiment of the present invention in step (a) 1-formyl-9H-β-carboline is reacted with nitromethane and NH₄OAc at 80-90° C. for 15 min, extracted with ethylacetate and subjected to column chromatography over silica gel to obtain 1-acetyl-9H-β-carboline.

The present invention also provides a pharmaceutical composition comprising of an effective amount of compound as for formula I as described herein, or a pharmaceutical acceptable salt thereof and a pharmaceutically acceptable carrier.

In a preferred embodiment of the present invention the pharmaceutically acceptable carrier is suitable for systemic or oral administration.

In one embodiment of the present invention the compounds as described herein, or the composition as described herein, for use in a method for preventing, ameliorating or treating a disease, disorder or syndrome. The disease, disorder or syndrome as described herein is neuropathic pain, visceral pain, drug addiction, hyperalgesia, arthritic inflammation, autoimmune inflammation or a combination thereof.

The present invention further provides a method for preventing, ameliorating or treating a disease, disorder or syndrome in a subject in need thereof comprising administering to the subject a therapeutically effective amount of the compounds as described herein or the composition as described herein. The disease, disorder or syndrome as described herein is neuropathic pain, visceral pain, drug addiction, hyperalgesia, arthritic inflammation, autoimmune inflammation, or a combination thereof.

The present invention further provides a use of a compounds as described herein, or the composition as described herein, in the manufacture of a medicament for the treatment of pain, substance abuse, and pruritus in a subject in need thereof, wherein the medicament is to be administered to the subject.

An embodiment of the present invention provides compounds of general formula I, their stereoisomers, their tautomeric forms, their enantiomers, or their pharmaceutically acceptable salt.

In further embodiment of the present invention is provided pharmaceutical composition containing compounds of general formula I, their stereoisomers, their tautomeric forms, their enantiomers, or their pharmaceutically acceptable salts or their mixtures in combination with suitable carriers, solvents, diluents and other media normally employed in preparing such compositions.

In still further embodiment is provided the use of compounds of the present invention as biased KOR agonist, by administering an effective and non-toxic amount of compound of general formula I.

Biased agonist of KOR which may be generally described as 2,11-dihydroimidazo[1′,5′:1,2]pyrido[3,4-b]indol-4-ium chloride and related compounds are provided herein

The invention is directed to the compounds belonging to the general formula I, their stereoisomers, their tautomeric forms, their enantiomers, or their pharmaceutically acceptable salt thereof having full and biased KOR agonist activity, which are useful for the treatment of pain with reduced adverse effect.

The present invention also provides process and intermediates for making the compounds of present invention or stereoisomers, tautomers, their enantiomers, or their pharmaceutically acceptable salt, solvates or prodrugs thereof.

The present invention also provides the methods for treating pain without any side-effect comprising administering to a host in a need of such treatment a therapeutically effective amount of at least one of the compounds of the present invention or stereoisomers, tautomers, enantiomers, or their pharmaceutically acceptable salt, solvates or prodrugs thereof.

The present invention also provides the compounds of present invention or stereoisomers, tautomers, their enantiomers, or their pharmaceutically acceptable salt, solvates or prodrugs thereof, for use in therapy.

The present invention also provides the compounds of present invention or stereoisomers, tautomers, their enantiomers, or their pharmaceutically acceptable salt, solvates or prodrugs thereof, for the manufacture of a medicament for the treatment of neuropathic pain without any severe side-effect.

### LIST OF ABBREVIATIONS

KOR—Kappa-opioid receptor
MOR—Mu-opioid receptor
DOR—Delta-opioid receptor
DMSO—Dimethyl sulphoxide

### BRIEF DESCRIPTION OF THE DRAWINGS

FIG. 1: Analgesic effect of 8abd in mice measured using tail flick assay

FIG. 2: Effect of 8abd on novelty induced locomotor in open field.

FIG. 3: Effect of 8abd on motor coordination as evaluated by rotarod test.

FIG. 4: Beneficial effect of8abd on neuropathic pain (mechanical and thermal hyperalgesia).

FIG. 5: Chronic effect of 8abd on Paclitaxel-induced peripheral neuropathic pain

FIG. 6: Behavioral effects of intracerebroventricular (i.e.v.) of 8abd

### DETAILED DESCRIPTION OF THE INVENTION

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

The present disclosure relates to compounds and to their use as selective agonists of the KOR. The disclosure also relates to methods for the preparation of these compounds and to pharmaceutical compositions containing such compounds. The compounds described herein relate to and/or have the application(s) in (among others) the fields of drug discovery, pharmacotherapy, physiology, organic chemistry.

The KOR agonist compounds of the general formula I represented below and include their pharmaceutically acceptable salts,

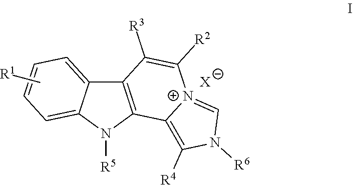

wherein
R¹ is selected from hydrogen, halogen, alkoxy, benzyloxy, sulphonic acid, sulphonamide, cyano, nitro or amide;
R² is selected from hydrogen, C₁₋₂ alkyl, alkyloycarbonyl or aryloxycarbonyl;
R³ is selected from hydrogen or aryl;
R² and R³ may jointly form an aromatic ring having substituent selected from hydrogen, trifluoromethyl, nitro or halogen;
R⁴ is selected from substituted or unsubstituted alkyl, aryl or heteroaryl, wherein the substitutents are selected from the groups consisting of, halogen, alkyl, aryl, alkoxy, aryloxy, thiomethyl, trifluoromethyl, trifluoromethyloxy, thioalkyl, sulphonylamide or nitro;
R⁵ is selected from H, alkyl, alkenyl, aryl, arylalkyl, heteroaryl, benzoyl, sulphonylphenyl, or alkoxycarbonyl;
R⁶ is selected from substituted or unsubstituted alkyl, aryl, arylalkyl, 5 or 6-membered heteroaryl, alkoxycarbonylalkyl, cycloalkyl, hetercycloalkyl, wherein the substitutents are selected from halogen, alkyl, alkoxy, aryl or alkyl aminoalkyl; and
X is selected from Cl, I, ClO₄, CF₃SO₂, BF₄ or PF₆.

In a preferred embodiment, the groups described above may be selected from:

“Alkyl”, as well as other groups having a prefix “alk”, such as alkoxy and alkanoyl, means carbon chain which may be linear or branched, and a combination thereof, unless the carbon chain is defined otherwise. Examples of alkyl group include but not limited to methyl ethyl, butyl, tert-butyl etc.

“Alkenyl” means carbon chains that contain at least one carbon-carbon double bond, which may be either linear or branched or combinations thereof, unless the carbon chain is defined otherwise. Examples of alkenyl include but not limited to vinyl, allyl, isopropenyl, 1-propenyl, 2-butenyl, 2-methyl-2-butenyl etc.

The “alkoxy” refers to the straight or branched chain alkoxides of the number of carbon atoms specified.

“Aryl” means a mono- or polycyclic aromatic ring system containing carbon ring atoms. The preferred aryls are monocyclic or bicyclic 6-10 membered aromatic ring systems. Phenyl and naphthyl are preferred aryls.

“Heteroaryl” refer to aromatic ring systems containing at least one heteroatom selected from O, S and N. Heteroaryl thus includes heteroaryls fused to other kinds of rings, such as aryls, cycloakyls, and heterocycles that not aromatic. Examples of heteroaryl include furanyl, benzofuranyl, thienyl, benzothienyl, pyridyl, pyrazolyl, oxazolyl, isoxazolyl, pyrrolyl, imidazolyl, benzisoxazolyl, benzoxazolyl, indazolyl, isoindolyl, indolinyl, pyradazinyl, carbazolyl, quinazolyl, indolyl, isoquinolyl etc.

Halogen refers to fluorine, chlorine, bromine and iodine. Bromine, chlorine and fluorine are generally preferred.

Suitable groups and substituents on the groups may be selected from those described anywhere in the specification.

The term “substituted,” as used herein, means that any one or more hydrogens on the designated atom is replaced with the selection from the indicated group, provided that the designated atom's normal valency is not exceeded, and that the substitution results into stable compound.

The term optional or optionally means that the subsequent described event or circumstance may or may not occur and the description includes instances in which it does not. For example, optionally substituted alkyl means either alkyl or substituted alkyl. Further, an optionally substituted group means unsubstituted.

Unless otherwise stated, stated in the specification, structures depicted herein are also meant to include compounds, which differ only in the presence of one or more isotopically enriched atom.

Particularly useful compounds may be selected from but not limited to
1. 1,2-Diphenyl-2,11-dihydroimidazo[1′,5′:1,2]pyrido[3,4-b]indol-4-ium chloride. (8aaa).
2. 2-(4-Bromophenyl)-1-phenyl-2,11-dihydroimidazo[1′,5′:1,2]pyrido[3,4-b]indol-4-ium chloride (8aab).
3. 2-(4-Chlorophenyl)-1-phenyl-2,11-dihydroimidazo[1′,5′:1,2]pyrido[3,4-b]indol-4-ium chloride (8aac).
4. 2-(4-Fluorophenyl)-1-phenyl-2,11-dihydroimidazo[1′,5′:1.2]pyrido[3,4-b]indol-4-ium chloride (8aad)
5. 2-(3-Bromophenyl)-1-phenyl-2,11-dihydroimidazo[1′,5′:1.2]pyrido[3,4-b]indol-4-ium chloride (8aae).
6. 2-(3-Chlorophenyl)-1-phenyl-2,11-dihydroimidazo[′,5′:1.2]pyrido[3,4-b]indol-4-ium chloride (8af).
7. 1-(4-Bromophenyl)-2-phenyl-2,11-dihydroimidazo[1′,5′:1.2]pyrido[3,4-b]indol-4-ium chloride (8aba).
8. 1,2-Bis(4-bromophenyl)-2,11-dihydroimidazo[1′,5′:1,2]pyrido[3,4-b]indol-4-ium chloride (8abb).
9. 1-(4-Bromophenyl)-2-(4-chlorophenyl)-2,11-dihydroimidazo[1′,5′:1,2]pyrido[3,4-b]indol-4-ium chloride (8abc).
10. 1-(4-Bromophenyl)-2-(4-fluorophenyl)-2,11-dihydroimidazo[1′,5′:1,2]pyrido[3,4-b]indol-4-ium chloride (8abd).
11. 2-(3-Bromophenyl)-1-(4-bromophenyl)-2,11-dihydroimidazo[1′,5′:1,2]pyrido[3,4-b]indol-4-ium chloride (8abe).
12. 1-(4-Bromophenyl)-2-(3-chlorophenyl)-2,11-dihydroimidazo[1′,5′:1,2]pyrido[3,4-b]indol-4-ium chloride (8abf).
13. 1-(4-Bromophenyl)-2-(4-methoxyphenyl)-2,11-dihydroimidazo[1′,5′:1,2]pyrido[3,4-b]indol-4-ium chloride (8abg).
14. 1-(4-Bromophenyl)-2-(4-(trifluoromethoxy)benzyl)-2,11dihydroimidazo[1′,5′:1,2]pyrido[3,4-b]indol-4-ium chloride (8abh).
15. 1-(4-Bromophenyl)-2-(4-5′:1,2]pyrido[3,4-b]indol-4-ium chloride (5abh). 1-(4-Bromophenyl)-2-(3-fluorophenyl)-2,11-dihydroimidazo[1′,5′:1,2]pyrido[3,4-b]indol-4-ium chloride (8abi).
16. 1-(4-Bromophenyl)-2-(2-chlorophenyl)-2,11-dihydroimidazo[1′,5′:1,2]pyrido[3,4-b]indol-4-ium (8abj).
17. 1-(4-Bromophenyl)-2-(2-fluorophenyl)-2,11-dihydroimidazo[1′,5′:1,2]pyrido[3,4-b]indol-4-ium chloride (8abk).
18. 1-(4-Bromophenyl)-2-(2-chloro-4-fluorophenyl)-2,11-dihydroimidazo[1′,5′:1,2]pyrido[3,4-b]indol-4-ium chloride (8abl).
19. 1-(4-Bromophenyl)-2-(3,4-difluorophenyl)-2,11-dihydroimidazo[1′,5′:1,2]pyrido[3,4-b]indol-4-ium chloride (8abm).
20. 1-(2-Bromophenyl)-2-phenyl-2,11-dihydroimidazo[1′,5′:1,2]pyrido[3,4-b]indol-4-ium chloride (8aca).
21. 1-(2-Bromophenyl)-2-(4-bromophenyl)-2,11-dihydroimidazo[1′,5′:1,2]pyrido[3,4-b]indol-4-ium chloride (8acb).
22. 1-(2-Bromophenyl)-2-(4-chlorophenyl)-2,11-dihydroimidazo[1′,5′:1,2]pyrido[3,4-b]indol-4-ium (acc).
23. 1-(2-Bromophenyl)-2-(4-fluorophenyl)-2,11-dihydroimidazo[1′,5′:1,2]pyrido[3,4-b]indol-4-ium chloride (8acd).
24. 1-(2-Bromophenyl)-2-(3-chlorophenyl)-2,11-dihydroimidazo[1′,5′:1,2]pyrido[3,4-b]indol-4-ium chloride (8acf).
25. 1-(2-Bromophenyl)-2-(3-fluorophenyl)-2,11-dihydroimidazo[1′,5′:1,2]pyrido[3,4-b]indol-4-ium (aci).
26. 2-(4-Bromophenyl)-1-(4-chlorophenyl)-2,11-dihydroimidazo[1′,5′:1,2]pyrido[3,4-b]indol-4-ium chloride (8adb).
27. 1,2-Bis(4-chlorophenyl)-2,11-dihydroimidazo[1′,5′:1,2]pyrido[3,4-b]indol-4-ium chloride (8adc).
28. 1-(4-Chlorophenyl)-2-(4-fluorophenyl)-2,11-dihydroimidazo[1′,5′:1,2]pyrido[3,4-b]indol-4-ium chloride (8add).
29. 2-(3-Bromophenyl)-1-(4-chlorophenyl)-2,11-dihydroimidazo[1′,5′:1,2]pyrido[3,4-b]indol-4-ium chloride (8ade).
30. 2-(3-Chlorophenyl)-1-(4-chlorophenyl)-2,11-dihydroimidazo[1′,5′:1,2]pyrido[3,4-b]indol-4-ium chloride (8adf).
31. 1-(4-Chlorophenyl)-2-(4-methoxyphenyl)-2,11-dihydroimidazo[1′,5′:1,2]pyrido[3,4-b]indol-4-ium chloride (8adg).
32. 1-(2-Chlorophenyl)-2-phenyl-2,11-dihydroimidazo[1′,5′:1,2]pyrido[3,4-b]indol-4-ium chloride (8aea).
33. 2-(4-Bromophenyl)-1-(2-chlorophenyl)-2,11-dihydroimidazo[1′,5′:1,2]pyrido[3,4-b]indol-4-ium chloride (8aeb).
34. 1-(2-Chlorophenyl)-2-(4-chlorophenyl)-2,11-dihydroimidazo[1′,5′:1,2]pyrido[3,4-b]indol-4-ium chloride (8aec).
35. 1-(2-Chlorophenyl)-2-(4-fluorophenyl)-2,11-dihydroimidazo[1′,5′:1,2]pyrido[3,4-b]indol-4-ium chloride (8aed).
36. 1-(3-Chlorophenyl)-2-(4-chlorophenyl)-2,11-dihydroimidazo[1′,5′:1,2]pyrido[3,4-b]indol-4-ium chloride (8afc).
37. 1-(3-chlorophenyl)-2-(4-fluorophenyl)-2,11-dihydroimidazo[1′,5′:1,2]pyrido[3,4-b]indol-4-ium chloride (8afd).
38. 1-(4-Fluorophenyl)-2-phenyl-2,11-dihydroimidazo[1′,5′:1,2]pyrido[3,4-b]indol-4-ium chloride (8aga).
39. 2-(4-Bromophenyl)-1-(4-fluorophenyl)-2,11-dihydroimidazo[1′,5′:1,2]pyrido[3,4-b]indol-4-ium chloride (8agb).
40. 2-(4-Chlorophenyl)-1-(4-fluorophenyl)-2,11-dihydroimidazo[1′,5′:1,2]pyrido[3,4-b]indol-4-ium chloride (8agc).
41. 1,2-Bis(4-fluorophenyl)-2,11-dihydroimidazo[1′,5′:1,2]pyrido[3,4-b]indol-4-ium chloride (8agd).
42. 2-(3-Bromophenyl)-1-(4-fluorophenyl)-2,11-dihydroimidazo[1′,5′:1,2]pyrido[3,4-b]indol-4-ium chloride (8age).
43. 2-(3-Chlorophenyl)-1-(4-fluorophenyl)-2,11-dihydroimidazo[1′,5′:1,2]pyrido[3,4-b]indol-4-ium chloride (8agf)
44. 1-(4-Bromophenyl)-2-(4-chlorophenyl)-2,11-dihydroimidazo[1′,5′:1,2]pyrido[3,4-b]indol-4-ium (8ahc).
45. 2-(4-Chlorophenyl)-1-(3-fluorophenyl)-2,11-dihydroimidazo[1′,5′:1,2]pyrido[3,4-b]indol-4-ium chloride (8aic).
46. 1-(3-Fluorophenyl)-2-(4-fluorophenyl)-2,11-dihydroimidazo[1′,5′:1,2]pyrido[3,4-b]indol-4-ium (8aid).
47. 1-(2-Bromo-4-fluorophenyl)-2-(4-chlorophenyl)-2,11-dihydroimidazo[1′,5′:1,2]pyrido[3,4-b]indol-4-ium (8ac).
48. 2-(4-Fluorophenyl)-1-(p-tolyl)-2,11-dihydroimidazo[1′,5′:1,2]pyrido[3,4-b]indol-4-ium chloride (8akd).
49. 2-(4-Fluorophenyl)-1-(4-nitrophenyl)-2,11-dihydroimidazo[1′,5′:1,2]pyrido[3,4-b]indol-4-ium chloride (8ald).
50. 1-(Benzofuran-2-yl)-2-phenyl-2,11-dihydroimidazo[1′,5′:1,2]pyrido[3,4-b]indol-4-ium chloride (8amd).
51. 2-(4-Fluorophenyl)-1-(thiophen-2-yl)-2,11-dihydroimidazo[1′,5′:1,2]pyrido[3,4-b]indol-4-ium chloride (8and).
52. 2-(4-Fluorophenyl)-1-(4-methoxyphenyl)-2,11-dihydroimidazo[1′,5′:1,2]pyrido[3,4-b]indol-4-ium (8aod).
53. 2-(4-fluorophenyl)-1-(3,4,5-trimethoxyphenyl)-2,11-dihydroimidazo[1′,5′:1,2]pyrido[3,4-b]indol-4-ium chloride (8apd).
54. 1-(4-Bromophenyl)-2-(4-chlorophenyl)-5-(methoxycarbonyl)-2,11-dihydroimidazo[1′,5′:1,2]pyrido[3,4-b]indol-4-iumchloride (8bbc)
55. 1-(4-Bromophenyl)-2-(4-fluorophenyl)-5-(methoxycarbonyl)-2,11-dihydroimidazo[1′,5′:1,2]pyrido[3,4-b]indol-4-iumchloride (8bbd)
56. 2-(4-Fluorophenyl)-5-(methoxycarbonyl)-1-(p-tolyl)-2,11-dihydroimidazo[1′,5′:1,2]pyrido[3,4-b]indol-4-iumchloride (8bkd).
57. 2-(4-Fluorophenyl)-5-(methoxycarbonyl)-1-methyl-2,11-dihydroimidazo[1′,5′:1,2]pyrido[3,4-b]indol-4-ium chloride (8bpd).
58. 8-Bromo-1-(2-bromophenyl)-2-phenyl-2,11-dihydroimidazo[1′,5′:1,2]pyrido[3,4-b]indol-4-ium chloride (8ccd).
59. 1-(4-Bromophenyl)-2-(4-chlorophenyl)-11-methyl-2,11 dihydroimidazo[1′,5′:1,2]pyrido[3,4-b]indol-4-iumchloride (8dbc)
60. 1-(4-Bromophenyl)-2-(4-fluorophenyl)-11-methyl-2,11-dihydroimidazo[1′,5′:1,2]pyrido[3,4-b]indol-4-iumchloride (8dbd).
61. 1,2-Bis(4-chlorophenyl)-11-methyl-2,11-dihydroimidazo[1′,5′:1,2]pyrido[3,4-b]indol-4-iumchloride (8ddc).
62. 2-Benzyl-1-(4-bromophenyl)-2,11-dihydroimidazo[1′,5′:1,2]pyrido[3,4-b]indol-4-ium chloride (9aaa).
63. 1-(4-Bromophenyl)-2-(4-fluorobenzyl)-2,11-dihydroimidazo[1′,5′:1,2]pyrido[3,4-b]indol-4-ium chloride (9aab).
64. 1-(4-Bromophenyl)-2-(4-(tert-butyl)benzyl)-2,11-dihydroimidazo[1′,5′:1,2]pyrido[3,4-b]indol-4-ium chloride (9aac).
65. 1-(4-Bromophenyl)-2-(4-(trifluoromethyl)benzyl)-2,11-dihydroimidazo[1′,5′:1,2]pyrido[3,4-b]indol-4-ium chloride (9aad).
66. 2-(4-Methoxybenzyl)-1-phenyl-2,11-dihydroimidazo[1′,5′:1,2]pyrido[3,4-b]indol-4-ium chloride (9aae).
67. 1-(4-Bromophenyl)-2-(2,5-difluorophenyl)-2,11-dihydroimidazo[1′,5′:1,2]pyrido[3,4-b]indol-4-ium chloride (9aaf)
68. 1-(4-Bromophenyl)-2-(3,4-dimethoxybenzyl)-2,11-dihydroimidazo[1′,5′:1,2]pyrido[3,4-b]indol-4-ium chloride (9aag)
69. 1-(4-Chlorophenyl)-2-(4-methoxybenzyl)-2,11-dihydroimidazo[1′,5′:1,2]pyrido[3,4-b]indol-4-ium chloride (9abe)
70. 1-(4-Chlorophenyl)-2-(4-(trifluoromethoxy)benzyl)-2,11-dihydroimidazo[1′,5′:1,2]pyrido[3,4-b]indol-4-ium chloride (9abh)
71. 1-(4-Chlorophenyl)-2-(3,4-dimethoxybenzyl)-2,11-dihydroimidazo[1′,5′:1,2]pyrido[3,4-b]indol-4-ium chloride (9abg)
72. 1-(4-Chlorophenyl)-2-(thiophen-3-ylmethyl)-2,11-dihydroimidazo[1′,5′:1,2]pyrido[3,4-b]indol-4-ium chloride (9acj).
73. 2-(4-Fluorophenyl)-1-phenyl-2,13-dihydroimidazo[1,5-a]indolo[2,3-c]quinolin-4-ium chloride (12ad)
74. 1-(4-Bromophenyl)-2-(4-chlorophenyl)-2,13-dihydroimidazo[1,5-a]indolo[2,3-c]quinolin-4-ium chloride (12bc)
75. 1-(4-Bromophenyl)-2-(4-fluorophenyl)-2,13-dihydroimidazo[1,5-a]indolo[2,3-c]quinolin-4-ium chloride (12bd)
76. 1,2-Bis(4-chlorophenyl)-2,13-dihydroimidazo[1,5-a]indolo[2,3-c]quinolin-4-ium chloride (12cc)
77. 2-(4-Bromophenyl)-1-(4-chlorophenyl)-2,13-dihydroimidazo[1,5-a]indolo[2,3-c]quinolin-4-ium chloride (12db)
78. 2-(4-Fluorophenyl)-1-(p-tolyl)-2,13-dihydroimidazo[1,5-a]indolo[2,3-c]quinolin-4-ium chloride (12dd)

The compounds of the present invention may be prepared using the reactions and techniques described below together with conventional techniques known to those skilled in the art of organic synthesis or variations thereof as appreciated by those skilled in the art. Compounds of the present invention can be isolated in the form of chloride salt. The compounds can be purified wherever required by recrystallization, trituration, column chromatography, Preparative thin layer chromatography, flash chromatography, or by preparative HPLC method. The reactions are performed in solvents appropriate to the reagents and materials employed and are suitable for the transformations being effected. Preferred methods include, but not limited to those described below.

Scheme A provides the general process for preparing the compound of formula I, wherein R¹, R², R³, R⁴, R⁵, R⁶ and X are as defined above.

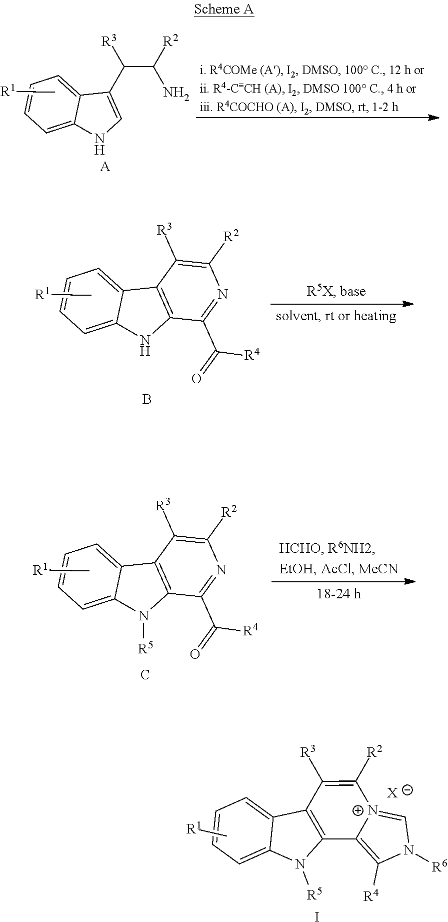

The first step of Scheme A involves reacting an indole derivative of formula A with a compound of formula A′ selected from arylpyruvaldehyde, terminal alkyne or substituted acetophene in the presence of iodine in DMSO as solvent for 1 to 5 h at a temperature from rt-110° C. to obtain a compound of formula B. Purifying the compound of formula B by column chromatography over silica gel using hexane:ethylacetate as eluent. Compound of formula B is reacted with R⁵X, wherein R⁵ is selected from H, alkyl, alkenyl, aryl, arylalkyl, heteroaryl, benzoyl, sulphonylphenyl, or alkoxycarbonyl and X is a halogen, in the presence of cesium carbonate in DMF and adding crushed ice to obtain solid precipitate of the N-substituted carboline compound of formula C. In the final step formaldehyde is reacted with the substituted β-carboline compound of formula B or C and amine of formula R⁶NH₂, wherein R⁶ is selected from substituted or unsubstituted alkyl, aryl, arylalkyl, 5 or 6-membered heteroaryl, alkoxycarbonylalkyl, cycloalkyl, or hetercycloalkyl, wherein the substitutents are selected from halogen, alkyl, alkoxy, aryl or alkyl aminoalkyl, to obtain compound for formula I.

The compounds of formula I can be prepared as described in Scheme A along with suitable modifications/variations, which are well within the scope of a person skilled in the art.

### Steps for Preparation of the Compounds

Step 1 The 2-(1H-indol-3-yl)ethan-1-amine 1 or alkyltryptophanate 2 or 2-(1H-indol-3-yl)aniline 3 can be reacted with acetophenone or substituted acetophenones 4 or terminal alkynes 5 in the presence of iodine and a suitable oxidant such as hydrogen peroxide or oxone under suitable condition of solvent and temperature to obtain the 1-aroyl-β-carbolines or (7H-benzo[c]carbazole-6-yl)(phenyl)methanone 7 (as solid or oil) as described in the prior art (Zhu, Y. P., Liu, M. C., Cai, Q., Jia, F. C., Wu. A. X. Chem. Eur. J. 2013, 19, 10132-10137; Dighe, S. U., Samanta, S. K., Kolle, S., Batra, S. Tetrahedron 2017, 73, 2455-2467; Chalotra, N.; Ahmed, A.; Rizvi, Masood A.; Hussain, Z.; Ahmed, Q. N.; Shah, B. A. J. Org. Chem. 2018, 83, 14443-14456) (Scheme 1). Alternatively, 1-3 may be treated with arylpyruvaldehyde 6 in the presence of iodine under suitable solvent and temperature to prepare 7 (as solid or oil).

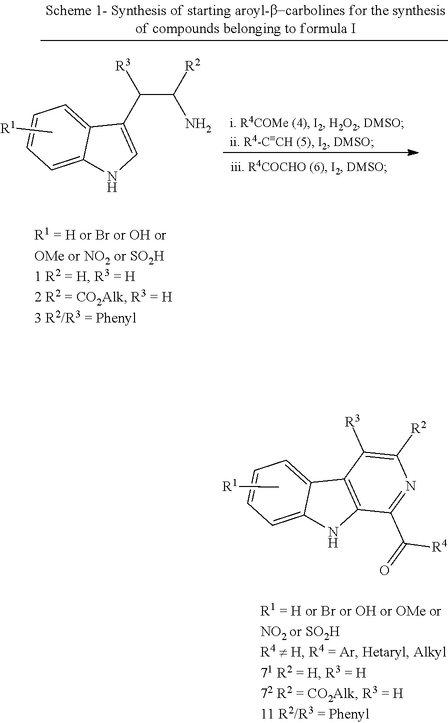

For preparing 1-acetyl-9H-β-carboline 7² (R⁴=Me), the 1-formyl-9H-β-carboline 7^{2′} (R⁴=H) prepared by the method described in the literature (Singh, V; Hutait, S; Batra, S. Eur. J. Org. Chem. 2009, 6211-6216) was treated with nitromethane in the presence of ammonium acetate under heating at a suitable temperature (Scheme 2).

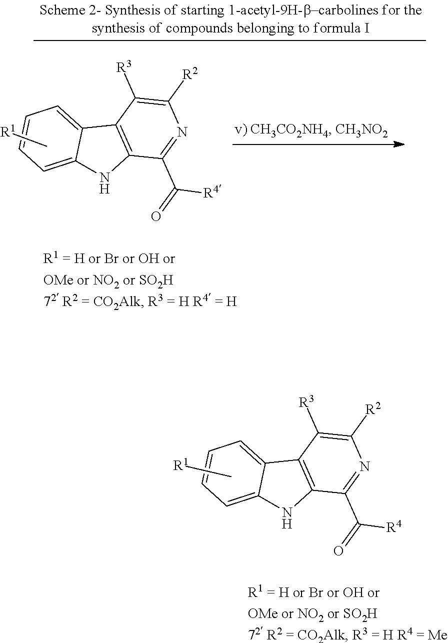

Step 2. For preparing N-substituted carbolines 10, compounds 7 were treated with suitable reagent from alkyl halide, alkylbromoacetate, arylmethylbromide, aroylbromide, phenylsulphonylchloride in the presence of appropriate bases and suitable solvent and temperature as described in prior art (Singh, M.; Jamra, R., Paul, A. K., Malakar, C. C.; Singh, V. Asian J. Org. Chem. 2022, 11, e202100653; Zhang, G.; Cao, R.; Guo, L.; Ma, Q.; Fan, W.; Chen, X.; Li, J.; Shao, G.; Qiu, L.; Ren, Z. Eur. J. Med Chem. 2013, 6S, 21-31) (Scheme 3)

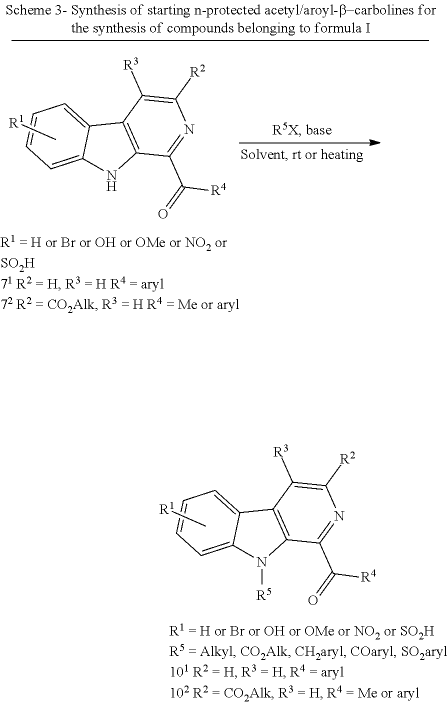

Step 3. Compound I was obtained via a Mannich reaction between formaldehyde, suitable amine and the appropriately substituted β-carboline derivative 7 or 10 or 11 under suitable solvent and temperature as described in the prior art with some modification (Dighe, S. U., Khan, S., Soni, I., Jain, P., Shukla, S., Yadav, R., Sen, P., Meeran, S. M., Batra, S. J. Med. Chem. 2015, 58, 3485-3499).

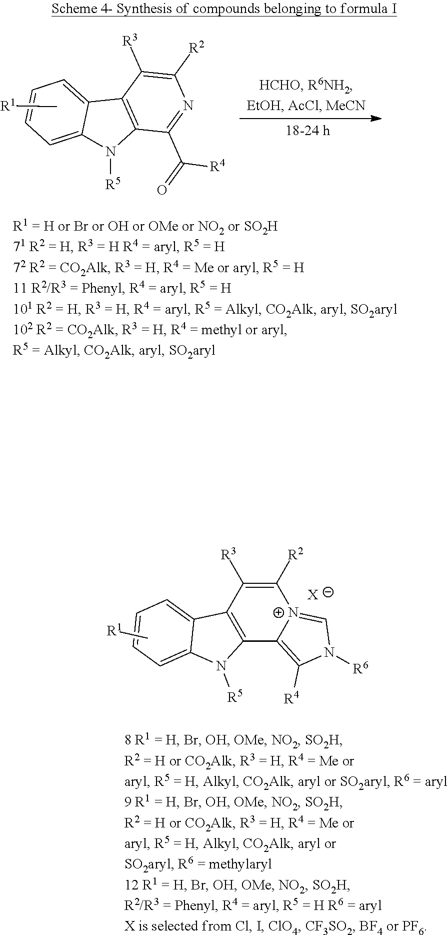

The examples and preparations provided below further illustrate and exemplify the compounds of the present invention and methods for preparing such compounds. It is to be understood that the scope of the present invention is not limited to anyway by the scope of the following examples and the preparation.

The invention is further illustrated by the following non-limiting examples, which describe the preferred way of carrying out the present invention. These are provided without limiting the scope

### Example 1

### Synthesis of compound 11-(4-Bromophenyl)-2-(4-fluorophenyl)-2,11-dihydroimidazo[1′,5′:1,2]pyrido[3,4-b]indol-4-ium chloride (8abd)

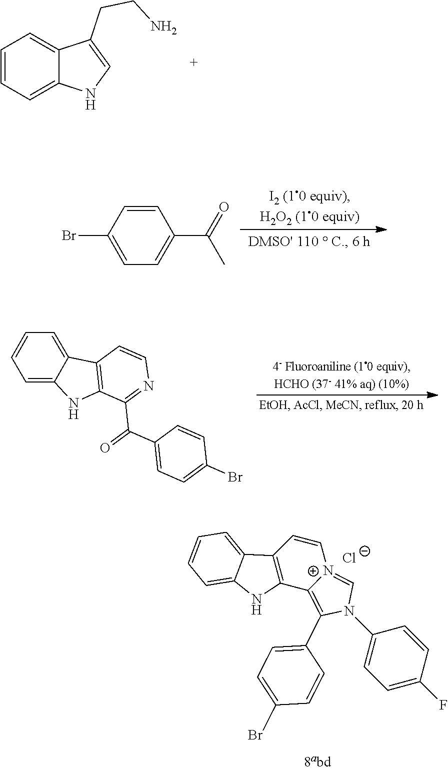

Step A. Hydrogen peroxide (30% aqueous solution, 2.19 mL, 93.75 mmol) was added to a mixture of tryptamine (12.30 g, 62.5 mmol), 4-bromoacetophenone (10 g, 62.5 mmol), and I₂ 60 (12.65 g, 50 mmol, 7.0 g) in DMSO (120 mL) at room temperature and the resulting mixture was stirred at 110° C. for 5 h and monitored via thin layer chromatography (TLC). On completion, the solution was cooled to room temperature, diluted with water (150 mL), and extracted with EtOAc (3×100 mL). The extract was washed with 10% Na₂S₂O₃ aqueous solution, dried over anhydrous Na₂SO₄, and concentrated under reduced pressure. The residue 65 was purified by column chromatography on silica gel (eluent: petroleum ether or hexanes/ethyl acetate, 8:2) to afford the desired product (4-bromophenyl)(9H-pyrido[3,4-b]indol-1-yl)methanone as yellow solid (17.02 g, 73.14%).

Step B. To a stirred solution of formaldehyde 37-41% solution in water (125 pL, 1.6 mmol) and 4-fluoroaniline (1.58 mL, 14.28 mmol) was added a freshly prepared ethanolic 70 hydrochloride (5.0 mL) following the literature procedure (Dighe, S. U., Khan, S., Soni, I., Jain, P., Shukla, S., Yadav, R., Sen, P., Meeran, S. M., Batra, S. J. Med. Chem. 2015, 58, 3485-3499) and the reaction was allowed to proceed for 20 min. A solution of (4-bromophenyl)(9H-pyrido[3,4-b]indol-1-yl)methanone (5.0 g, 14.28 mmol) in acetonitrile (60 mL) was added and the reaction was heated at 45° C. until it was dissolved and then continued for 18 h at room 75 temperature. After completion of the reaction, as monitored by TLC, the solvent was evaporated under reduced pressure to afford the crude product, which was purified by column chromatography (MeOH/EtOAc, 3:7) to afford 5.2 g (74.13%) as a white solid. The product was recrystallized by triturating with diethyl ether/ethanol mixture to give pure solid product. Mp: 258-260° C.; R_{f}=0.47 (MeOH:EtOAc, 5:5. v/v); IR (KBr) νₘₐₓ: 3454 cm⁻¹. ¹H NMR (400 MHz, DMSO-d⁶): δ (ppm)=7.32 (t, J=7.6 Hz, 1H), 7.41-7.49 (m, 5H), 7.64-7.68 (m, 3H), 7.76-7.75 (m, 1H), 7.79 (s, 1H), 8.17-8.20 (m, 2H), 8.58 (d, J=7.2 Hz, 1H), 10.63 (s, 1H), 11.41 (s, 1H). ¹³C NMR (100 MHz, DMSO-d⁶): δ (ppm)=113.5, 113.9, 114.1, 116.7, 117.1, 117.3, 120.3, 120.5, 121.5, 122.5, 122.6, 124.5, 124.6, 126.1, 129.7, 129.9, 130.1, 130.4, 133.1, 133.6, 139.4, 163.2 (d, J=250 Hz). MS (ESI+): m/z=456.0. ESI-HR-MS calculated for C₂₅H₁₆BrFN₃[M⁺]: 456.0506 (Br=79), found: 456.0514.

Following compounds can be readily prepared via an identical experimental procedure

1,2-Diphenyl-2,11-dihydroimidazo[1′,5′:1,2]pyrido[3,4-b]indol-4-ium chloride. (8aaa). Yield: 68.3% (0.32 g from 0.3 g); a light yellow solid; mp: 256-258° C.; R_{f}=0.59 (MeOH:EtOAc, 5:5. v/v); IR (KBr) νₘₐₓ: 3373 cm⁻¹. ¹H NMR (400 MHz, DMSO-d⁶): δ (ppm)=7.31 (t, J=7.3 Hz, 1H), 7.42 (t, J=7.4 Hz, 1H), 7.48-7.54 (m, 5H), 7.58 (t, J=6.8 Hz, 3H), 7.66 (d, J=8.0 Hz, 1H), 7.80 (d, J=7.5 Hz, 1H), 7.88-7.92 (m, 1H), 8.18-8.19 (m, 2H), 8.54 (d, J=7.1 Hz, 1H), 10.58 (s, 1H), 11.31 (s, 1H). ¹³C NMR (100 MHz, DMSO-d⁶): δ (ppm)=113.6, 113.8, 113.9, 116.6, 120.1, 120.2, 121.5, 122.0, 122.6, 123.3, 124.8, 124.9, 125.9, 126.6, 129.5, 129.9, 130.3, 130.9, 131.5, 131.7, 133.9, 139.3. MS (ESI+): m/z=360.1. ESI-HR-MS calcd for C₂₅H₁₈N₃ [M⁺]: 360.1501, found: 360.1509.

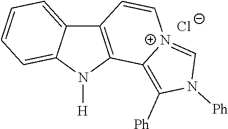 [SMILES: c1ccc(-c2c3c4[nH]c5ccccc5c4cc[n+]3cn2-c2ccccc2)cc1 ; [Cl-]]

2-(4-Bromophenyl)-1-phenyl-2,11-dihydroimidazo[1′,5′:1,2]pyrido[3,4-b]indol-4-ium chloride 8aab. Yield: 60% (0.313 g from 0.3 g); a white solid; mp: 301-303° C.; R_{f}=0.45 (MeOH:EtOAc, 5:5. v/v); IR (KBr) νₘₐₓ: 3401 cm⁻¹. ¹H NMR (400 MHz, DMSO-d⁶): δ (ppm)=7.31 (t, J=7.7 Hz, 1H), 7.42 (t, J=7.6 Hz, 1H), 7.50-7.51 (m, 4H), 7.55-7.61 (m, 3H), 7.67 (d, J=8.2 Hz, 1H), 7.79 (d, J=8.6 Hz, 2H), 8.16-8.19 (m, 2H), 8.56 (d, J=7.1 Hz, 1H), 10.61 (s, 1H). ¹³C NMR (100 MHz, DMSO-d⁶): δ (ppm)=113.7, 113.9, 116.7, 120.1, 120.4, 121.5, 122.7, 123.4, 124.4, 125.1, 126.0, 129.4, 129.5, 129.9, 130.9, 131.5, 133.1, 133.6, 139.4. MS (ESI+): m/z=438.0. ESI-HR-MS calcd for C₂₅H₁₇BrN₃[M⁺]: 438.0600, found: 438.0609.

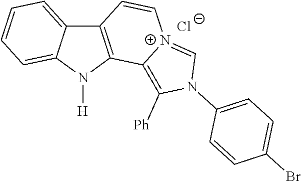 [SMILES: Brc1ccc(-n2c[n+]3ccc4c5ccccc5[nH]c4c3c2-c2ccccc2)cc1 ; [Cl-]]

2-(4-Chlorophenyl)-1-phenyl-2,11-dihydroimidazo[1′,5′:1,2]pyrido[3,4-b]indol-4-ium chloride (8aac). Yield: 78% (0.369 g from 0.3 g); a light green solid; mp: 297-301° C.; R_{f}=0.47 (MeOH:EtOAc, 5:5. v/v); IR (KBr) νₘₐₓ: 3409 cm⁻¹. ¹H NMR (400 MHz, DMSO-d⁶): δ (ppm)=7.31 (t, J=7.2 Hz, 1H), 7.42 (t, J=7.2 Hz, 1H), 7.52 (d, J=7.2 Hz, 2H), 7.56-7.60 (m, 5H), 7.65-7.68 (m, 3H), 8.17-8.20 (m, 2H), 8.59 (d, J=7.1 Hz, 1H), 10.65 (s, 1H), 11.30 (s, 1H). ¹³C NMR (100 MHz, DMSO-d⁶): δ (ppm)=118.4, 118.7, 118.8, 124.9, 125.1, 126.3, 127.4, 128.2, 129.7, 129.9, 130.7, 133.2, 133.9, 134.1, 134.6, 134.7, 134.8, 135.6, 136.3, 137.9, 140.5, 144.1. MS (ESI+): m/z=394.1. ESI-HR-MS calcd for C₂₅H₁₇ClN₃[M⁺]: 394.1106, found: 394.1108.

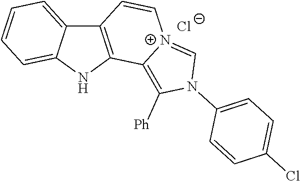 [SMILES: Clc1ccc(-n2c[n+]3ccc4c5ccccc5[nH]c4c3c2-c2ccccc2)cc1 ; [Cl-]]

2-(4-Fluorophenyl)-1-phenyl-2,11-dihydroimidazo[1′,5′:1,2]pyrido[3,4-b]indol-4-ium chloride (8aad). Yield: 79% (0.359 g from 0.3 g); a white solid; mp: 315-317° C.; R_{f}=0.42 (MeOH:EtOAc, 5:5. v/v); IR (KBr) νₘₐₓ: 3388 cm⁻¹. ¹H NMR (400 MHz, DMSO-d⁶): δ (ppm)=7.32 (t, J=7.6 Hz, 1H), 7.40-7.46 (m, 3H), 7.52 (d, J=6.5 Hz, 2H), 7.58 (d, J=7.5 Hz, 3H), 7.62-7.68 (m, 3H), 8.18 (t, J=6.6 Hz, 2H), 8.53 (d, J=7.2 Hz, 1H), 10.51 (s, 1H), 11.32 (s, 1H). ¹³C NMR (100 MHz, DMSO-d⁶): δ (ppm)=113.7, 113.9, 116.7, 116.9, 117.1, 120.2, 120.3, 121.5, 122.6, 123.7, 125.0, 125.2, 126.0, 129.5, 129.9, 130.0, 130.1, 130.6, 130.9, 131.5, 139.4, 163.1 (d, J=190 Hz). MS (ESI+): m/z=378.1. ESI-HR-MS calcd for C₂₅H₁₇FN₃ [M⁺]: 378.1401, found: 378.1400.

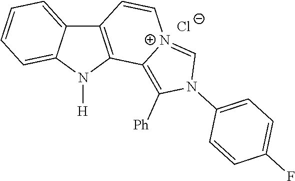 [SMILES: Fc1ccc(-n2c[n+]3ccc4c5ccccc5[nH]c4c3c2-c2ccccc2)cc1 ; [Cl-]]

2-(3-Bromophenyl)-1-phenyl-2,11-dihydrolmidazo[1′,5′:1,2]pyrido[3,4-b]indol-4-ium chloride (8aae). Yield: 68% (0.354 g from 0.3 g); a white solid; mp: 251-253° C.; R_{f}=0.48 (MeOH:EtOAc, 5:5. v/v), IR (KBr) μₘₐₓ: 3381 cm⁻¹. ¹H NMR (400 MHz, DMSO-d⁶): δ (ppm)=7.31 (t, J=7.4 Hz, 1H), 7.42 (t, J=7.7 Hz, 1H), 7.50-7.54 (m, 4H), 7.59 (d, J=6.0 Hz, 3H), 7.67 (d, J=8.3 Hz, 1H), 7.81 (d, J=7.3 Hz, 1H), 7.89-7.93 (m, 1H), 8.19 (d, J=5.7 Hz, 2H), 8.58 (d, J=7.0 Hz, 1H), 10.67 (s, 1H), 11.31 (s, 1H). ¹³C NMR (100 MHz, DMSO-d⁶): δ (ppm)=113.7, 113.9, 116.7, 120.2, 120.3, 121.5, 122.1, 122.6, 123.4, 124.9, 125.0, 126.0, 126.7, 129.6, 130.0, 130.3, 131.0, 131.6, 131.8, 134.0, 135.5, 139.4. MS (ESI+): m/z=438.0. ESI-HR-MS calcd for C₂₅H₁₇BrN₃[M⁺]: 438.0600, found: 438.0602.

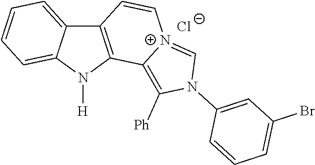 [SMILES: Brc1cccc(-n2c[n+]3ccc4c5ccccc5[nH]c4c3c2-c2ccccc2)c1 ; [Cl-]]

2-(3-Chlorophenyl)-1-phenyl-2,11-dihydroimidazo[1′,5′:1,2]pyrido[3,4-b]indol-4-ium chloride (8aaf). Yield: 70% (0.331 g from 0.3 g); a light yellow solid; mp: 230-232° C.; R_{f}=0.47 MeOH:EtOAc 5:5. v/v); IR (KBr) μₘₐₓ: 3355 cm⁻¹. ¹H NMR (400 MHz, DMSO-d⁶): δ (ppm)=7.29 (t, J=7.4 Hz, 1H), 7.40 (t, J=7.3 Hz, 1H), 7.52-7.59 (m, 6H), 7.65-7.68 (m, 3H), 7.77 (s, 1H), 8.17 (d, J=7.2 Hz, 2H), 8.59 (d, J=7.0 Hz, 1H), 10.74 (s, 1H). ¹³C NMR (100 MHz, DMSO-d⁶): δ (ppm)=113.8, 113.9, 114.0, 116.6, 120.2, 120.4, 121.4, 122.7, 123.4, 125.1, 125.9, 126.4, 127.6, 129.6, 129.9, 130.9, 131.1, 131.6, 131.7, 133.9, 135.5, 139.6. MS (ESI+): m/z=394.1. ESI-HR-MS calcd for C₂₅H₁₇ClN₃[M⁺]: 394.1106, found: 394.1108.

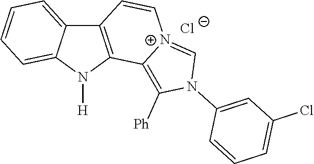 [SMILES: Clc1cccc(-n2c[n+]3ccc4c5ccccc5[nH]c4c3c2-c2ccccc2)c1 ; [Cl-]]

1-(4-Bromophenyl)-2-phenyl-2,11-dihydroimidazo[1′,5′:1,2]pyrido[3,4-b]indol-4-ium chloride (8aba). Yield: 78% (0.316 g from 0.3 g); a light yellow solid; mp: 230-232° C.; R_{f}=0.44 MeOH:EtOAc 5:5. v/v); IR (KBr) μₘₐₓ: 3450 cm⁻¹. ¹H NMR (400 MHz, DMSO-d⁶): δ (ppm)=7.31 (t, J=7.4 Hz, 1H), 7.42 (t, J=7.5 Hz, 1H), 7.48-7.54 (m, 4H), 7.58 (d, J=7.2 Hz, 3H), 7.66 (d, J=8.1 Hz, 1H), 7.80 (d, J=7.3 Hz, 1H), 7.88-7.92 (m, 1H), 8.17-8.20 (m, 2H), 8.55 (d, J=7.1 Hz, 1H), 10.61 (s, 1H), 11.31 (s, 1H). ¹³C NMR (100 MHz, DMSO-d⁶): δ (ppm)=113.7, 114.1, 116.6, 120.2, 120.3, 121.6, 122.3, 122.7, 123.5, 124.9, 126.1, 126.7, 129.5, 129.9, 130.3, 130.4, 131.6, 131.8, 132.5. MS (ESI+): m/z=438.0. ESI-HR-MS calcd for C₂₅H₁₇BrN₃[M⁺]: 438.0600, found: 438.0601.

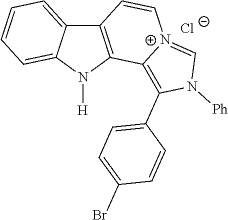 [SMILES: Brc1ccc(-c2c3c4[nH]c5ccccc5c4cc[n+]3cn2-c2ccccc2)cc1 ; [Cl-]]

1,2-Bis(4-bromophenyl)-2,11-dihydroimidazo[1′,5′:1,2]pyrido[3,4-b]indol-4-ium chloride (8abb). Yield: 62% (0.292 g from 0.3 g); a light yellow solid; mp: 280-282° C.; R_{f}=0.51 MeOH:EtOAc 5:5. v/v); IR (KBr) μₘₐₓ: 3395 cm⁻¹. ¹H NMR (400 MHz, DMSO-d⁶): δ (ppm)=7.33 (t, J=7.3 Hz, 1H), 7.43-7.53 (m, 4H), 7.65 (t, J=8.1 Hz, 2H), 7.80 (d, J=8.2 Hz, 3H), 7.95-8.03 (m, 1H), 8.19-8.20 (m, 2H), 8.48 (d, J=6.6 Hz, 1H), 10.42 (d, J=11.3 Hz, 1H), 11.39 (s, 1H). ¹³C NMR (100 MHz, DMSO-d⁶): δ (ppm)=113.5, 114.1, 116.7, 120.3, 121.6, 122.2, 122.3, 122.6, 124.3, 124.8, 125.0, 126.2, 126.8, 129.6, 129.9, 131.1, 133.1, 133.6, 139.4. MS (ESI+): m/z=515.9. ESI-HR-MS calcd for C₂₅H₁₆Br₂N₃+ [M⁺]: 515.9705, found: 515.9711.

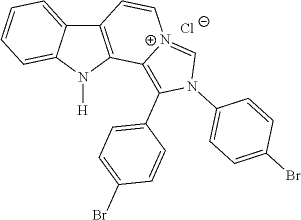 [SMILES: Brc1ccc(-c2c3c4[nH]c5ccccc5c4cc[n+]3cn2-c2ccc(Br)cc2)cc1 ; [Cl-]]

1-(4-Bromophenyl)-2-(4-chlorophenyl)-2,11-dihydromidazo[1′,5′:1,2]pyrido[3,4-b]indol-4-ium chloride (8abc). Yield: 86% (0.372 g from 0.3 g); a white solid; mp: 291-292° C.; R_{f}=0.45 (MeOH:EtOAc, 5:5. v/v); IR (KBr) νₘₐₓ: 3391 cm⁻¹. ¹H NMR (400 MHz, DMSO-d⁶): δ (ppm)=7.27 (t, J=7.3 Hz, 1H), 7.39 (t, J=7.2 Hz, 1H), 7.47 (d, J=8.4 Hz, 2H), 7.58-7.61 (m, 2H), 7.63 (d, J=8.2 Hz, 1H), 7.68-7.71 (m, 2H), 7.77 (d, J=8.5 Hz, 2H), 8.14-8.17 (m, 2H), 8.43 (t, J=6.4 Hz, 1H), 10.41 (s, 1H). ¹³C NMR (100 MHz, DMSO-d⁶): δ (ppm)=113.6, 114.1, 116.6, 120.3, 120.7, 121.5, 122.3, 122.7, 124.5, 124.9, 126.1, 129.4, 129.6, 130.2, 133.0, 133.1, 133.6, 135.9, 139.6. MS (ESI+): m/z=472.0. ESI-HR-MS calcd for C₂₅H₁₆BrClN₃[M⁺+H]: 472.0211, found: 472.0215.

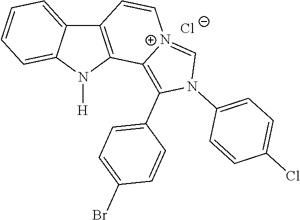 [SMILES: Clc1ccc(-n2c[n+]3ccc4c5ccccc5[nH]c4c3c2-c2ccc(Br)cc2)cc1 ; [Cl-]]

1-(4-Bromophenyl)-2-(4-fluorophenyl)-2,11-dihydrolmidazo[1′,5′:1,2]pyrido[3,4-b]indol-4-ium chloride (8abd). Yield: 81% (0.341 g from 0.3 g); a white solid; mp: 258-260° C.; R_{f}=0.47 (MeOH:EtOAc, 5:5. v/v); IR (KBr) νₘₐₓ: 3454 cm⁻¹. ¹H NMR (400 MHz, DMSO-d⁶): (ppm)=7.32 (t, J=7.6 Hz, 1H), 7.41-7.49 (m, 5H), 7.64-7.68 (m, 3H), 7.76-7.75 (m, 1H), 7.79 (s, 1H), 8.17-8.20 (m, 2H), 8.58 (d, J=7.2 Hz, 1H), 10.63 (s, 1H), 11.41 (s, 1H). ¹³C NMR (100 MHz, DMSO-d⁶): δ (ppm)=113.5, 113.9, 114.1, 116.7, 117.1, 117.3, 120.3, 120.5, 121.5, 122.5, 122.6, 124.5, 124.6, 126.1, 129.7, 129.9, 130.1, 130.4, 133.1, 133.6, 139.4, 163.2 (d, J=250 Hz). MS (ESI+): m/z=456.0. ESI-HR-MS calculated for C₂₅H₁₆BrFN₃[M⁺]: 456.0506, found: 456.0512.

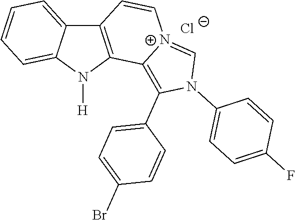 [SMILES: Fc1ccc(-n2c[n+]3ccc4c5ccccc5[nH]c4c3c2-c2ccc(Br)cc2)cc1 ; [Cl-]]

2-(3-Bromophenyl)-1-(4-bromophenyl)-2,11-dihydroimidazo[1′,5′:1,2]pyrido[3,4-b]indol-4-ium chloride (8abe). Yield: 68% (0.320 g from 0.3 g); a white solid; mp: 230-232° C.; R_{f}=0.59 (MeOH:EtOAc, 5:5. v/v); IR (KBr) νₘₐₓ: 3393 cm⁻¹. ¹H NMR (400 MHz, DMSO-d⁶): δ (ppm)=7.27 (s, 1H), 7.37-7.43 (m, 5H), 7.52-7.55 (m, 1H), 7.69 (s, 4H), 8.08 (s, 2H), 8.51 (s, 1H), 10.06 (s, 1H), 11.30 (s, 1H). ¹³C NMR (100 MHz, DMSO-d⁶): δ (ppm)=113.5, 114.0, 114.1, 116.7, 120.3, 120.6, 121.5, 122.2, 122.6, 124.4, 124.6, 124.8, 124.9, 126.1, 129.6, 133.1, 133.2, 133.4, 133.6, 139.4. MS (ESI+): m/z=515.9. ESI-HR-MS calcd for C₂₅H₁₆Br₂N₃ [M⁺]: 515.9705, found: 515.9710.

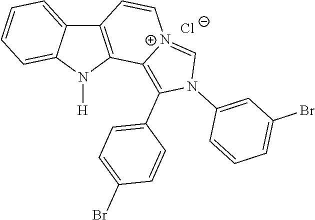 [SMILES: Brc1ccc(-c2c3c4[nH]c5ccccc5c4cc[n+]3cn2-c2cccc(Br)c2)cc1 ; [Cl-]]

1-(4-Bromophenyl)-2-(3-chlorophenyl)-2,11-dihydroimidazo[1′,5′:1,2]pyrido[3,4-b]indol-4-ium chloride (8abf). Yield: 71% (0.307 g from 0.3 g); a brown solid; mp: 272-274° C.; R_{f}=0.45 (MeOH:EtOAc, 5:5. v/v); IR (KBr) νₘₐₓ: 3018 cm⁻¹. ¹H NMR (400 MHz, DMSO-d⁶): δ (ppm)=7.27 (t, J=7.4 Hz, 1H), 7.39 (t, J=7.2 Hz, 1H), 7.47 (d, J=8.5 Hz, 2H), 7.59 (d, J=8.8 Hz, 2H), 7.64 (d, J=8.4 Hz, 1H), 7.67-7.71 (m, 2H), 7.58-7.78 (m, 2H), 8.13-8.17 (m, 2H), 8.37 (s, 1H), 8.45 (d, J=7.0 Hz, 1H), 10.44 (s, 1H). ¹³C NMR (100 MHz, DMSO-d⁶): δ (ppm)=113.6, 114.0, 116.5, 120.2, 120.7, 121.5, 122.3, 122.7, 124.5, 124.8, 125.0, 126.1, 129.4, 129.6, 130.2, 133.0, 133.1, 133.6, 135.9, 139.6. MS (ESI+): m/z=472.0. ESI-HR-MS calcd for C₂₅H₁₆BrClN₃ [M⁺]: 472.0211, found: 472.0216.

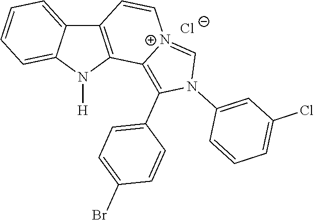 [SMILES: Clc1cccc(-n2c[n+]3ccc4c5ccccc5[nH]c4c3c2-c2ccc(Br)cc2)c1 ; [Cl-]]

1-(4-Bromophenyl)-2-(4-methoxyphenyl)-2,11-dihydrolmidazo[1′,5′:1,2]pyrido[3,4-b]indol-4-ium chloride (8abg). Yield: 78% (0.336 g from 0.3 g); a white solid; mp: 273-275° C.; R_{f}=0.44 (MeOH:EtOAc, 5:5. v/v); IR (KBr) νₘₐₓ: 3363 cm⁻¹. ¹H NMR (400 MHz, DMSO-d⁶): δ (ppm)=3.82 (S, 3H), 7.11 (s, 2H), 7.32-7.48 (m, 6H), 7.67-7.77 (m, 3H), 8.18 (s, 2H), 8.58 (s, 1H), 10.61 (s, 1H), 11.38 (s, 1H). ¹³C NMR (100 MHz, DMSO-d⁶): δ (ppm)=113.5, 113.8, 115.2, 116.8, 120.2, 121.5, 122.5, 124.7, 124.8, 124.9, 126.1, 126.7, 128.8, 129.6, 133.0, 133.6, 133.7, 139.4, 139.5, 160.9. MS (ESI+): m/z=468.0. ESI-HR-MS calcd for C₂₆H₁₉BrN₃O [M⁺]: 468.0706, found: 468.0711.

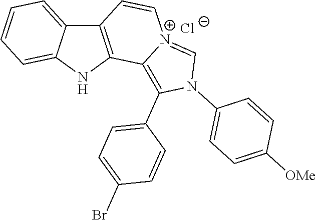 [SMILES: COc1ccc(-n2c[n+]3ccc4c5ccccc5[nH]c4c3c2-c2ccc(Br)cc2)cc1 ; [Cl-]]

1-(4-Bromophenyl)-2-(4-(trifluoromethoxy)benzyl)-2,11-dihydroimidazo[1′,5′:1,2]pyrido[3,4-b]indol-4-ium chloride (8abh). Yield: 81% (0.372 g from 0.3 g); a white solid; mp: 288-290° C.; R_{f}=0.42 (MeOH:EtOAc, 5:5. v/v); IR (KBr) νₘₐₓ: 3431 cm⁻¹. ¹H NMR (400 MHz, DMSO-d⁶): δ (ppm)=7.28 (t, J=7.5 Hz, 1H), 7.39 (t, J=7.8 Hz, 3H), 7.56 (d, J=8.4 Hz, 2H), 7.61 (d, J=8.2 Hz, 1H), 7.66 (d, J=8.1 Hz, 2H), 7.81 (d, J=8.3 Hz, 2H), 8.08 (d, J=7.2 Hz, 1H), 8.15 (d, J=7.9 Hz, 1H), 8.55 (d, J=7.2 Hz, 1H), 10.41 (s, 1H), 11.29 (s, 1H). ¹³C NMR (100 MHz, DMSO-d⁶): δ (ppm)=113.3, 113.4, 113.9, 117.1, 120.2, 121.1, 121.4, 121.5, 122.6, 123.1, 124.4, 124.9, 125.2, 125.8, 125.9, 126.0, 126.1, 128.8, 129.1, 129.2, 129.5, 131.3, 133.2, 133.6, 139.1, 139.3. MS (ESI+): m/z=522.0. ESI-HR-MS calcd for C₂₆H₁₆BrF₃N₃O [M⁺]: 522.0423, found: 522.0429.

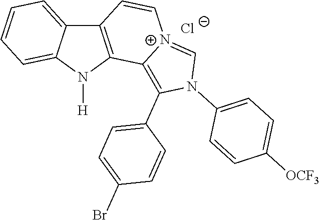 [SMILES: FC(F)(F)Oc1ccc(-n2c[n+]3ccc4c5ccccc5[nH]c4c3c2-c2ccc(Br)cc2)cc1 ; [Cl-]]

1-(4-Bromophenyl)-2-(3-fluorophenyl)-2,11-dihydroimidazo[1′,5′:1,2]pyrido[3,4-b]indol-4-ium chloride (8abi). Yield: 73% (0.307 g from 0.3 g); a brown solid; mp: 261-263° C.; R_{f}=0.43 (MeOH:EtOAc, 5:5. v/v); IR (KBr) νₘₐₓ: 3390 cm⁻¹. ¹H NMR (400 MHz, DMSO-d⁶): δ (ppm)=7.31 (t, J=7.5 Hz, 1H), 7.41-7.48 (m, 3H), 7.53 (d, J=7.2 Hz, 2H), 7.63-7.67 (m, 5H), 8.17-8.20 (m, 2H), 8.59 (d, J=7.2 Hz, 1H), 10.64 (s, 1H), 11.44 (s, 1H). ¹³C NMR (100 MHz, DMSO-d⁶): δ (ppm)=113.5, 114.0, 116.7, 117.0, 117.3, 120.3, 120.6, 121.5, 122.5, 122.7, 124.1, 124.8, 126.1, 129.6, 129.9, 130.0, 130.1, 130.4, 133.4, 136.0, 163.3 (d, J=300 Hz). MS (ESI+): m/z=456.0. ESI-HR-MS calcd for C₂₅H₁₆BrFN₃[M⁺]: 456.0506, found: 456.0502.

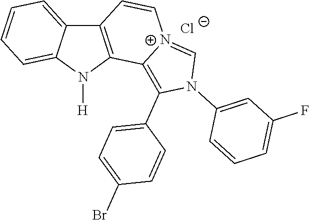 [SMILES: Fc1cccc(-n2c[n+]3ccc4c5ccccc5[nH]c4c3c2-c2ccc(Br)cc2)c1 ; [Cl-]]

1-(4-Bromophenyl)-2-(2-chlorophenyl)-2,11-dihydroimidazo[1′,5′:1,2]pyrido[3,4-b]indol-4-ium (8abj). Yield: 82% (0.355 g from 0.3 g); a light brown solid; mp: 280-282° C.; R_{f}=0.45 (MeOH:EtOAc, 5:5. v/v); IR (KBr) νₘₐₓ: 3371 cm⁻¹. ¹H NMR (400 MHz, DMSO-d⁶): δ (ppm)=7.31 (t, J=7.2 Hz, 1H), 7.39-7.47 (m, 4H), 7.57-7.60 (m, 2H), 7.64-7.67 (m, 4H), 8.16-8.19 (m, 2H), 8.56 (d, J=7.1 Hz, 1H), 10.59 (s, 1H), 11.38 (s, 1H). ¹³C NMR (100 MHz, DMSO-d⁶): δ (ppm)=113.5, 113.9, 114.1, 116.7, 117.0, 117.3, 120.4, 121.4, 121.6, 122.6, 126.1, 126.3, 129.0, 129.4, 129.5, 129.7, 133.8, 135.2. MS (ESI+): m/z=472.0. ESI-HR-MS calcd for C₂₅H₁₆BrClN₃[M⁺]: 472.0211, found: 472.0218.

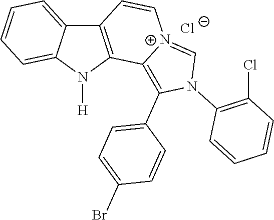 [SMILES: Clc1ccccc1-n1c[n+]2ccc3c4ccccc4[nH]c3c2c1-c1ccc(Br)cc1 ; [Cl-]]

1-(4-Bromophenyl)-2-(2-fluorophenyl)-2,11-dihydroimidazo[1′,5′:1,2]pyrido[3,4-b]indol-4-ium chloride (8abk). Yield: 71% (0.298 g from 0.3 g); a light green solid; mp: 295-297° C.; R_{f}=0.43 (MeOH:EtOAc, 5:5. v/v); IR (KBr) νₘₐₓ: 3403 cm⁻¹. ¹H NMR (400 MHz, DMSO-d⁶): δ (ppm)=7.21 (d, J=7.2 Hz, 1H), 7.32-7.39 (m, 5H), 7.54-7.66 (m, 4H), 7.74 (t, J=6.2 Hz, 1H), 8.08 (d, J=6.0 Hz, 2H), 8.48 (d, J=5.6 Hz, 1H), 10.59 (s, 1H), 11.63 (s, 1H). ¹³C NMR (100 MHz, DMSO-d⁶): δ (ppm)=113.4, 114.1, 116.8, 119.6, 120.4, 121.6, 122.6, 123.9, 124.5, 124.6, 126.2, 128.7, 128.8, 129.8, 130.8, 133.1, 133.4, 133.7, 135.0, 135.6, 139.6. MS (ESI+): m/z=456.0. ESI-HR-MS calcd for C₂₅H₁₆BrFN₃[M⁺]: 456.0506, found: 456.0510.

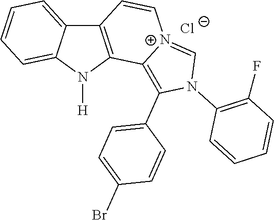 [SMILES: Fc1ccccc1-n1c[n+]2ccc3c4ccccc4[nH]c3c2c1-c1ccc(Br)cc1 ; [Cl-]]

1-(4-Bromophenyl)-2-(2-chloro-4-fluorophenyl)-2,11-dihydrolmidazo[1′,5′:1,2]pyrido[3,4-b]indol-4-ium chloride (8abl). Yield: 62% (0.278 g from 0.3 g); a light yellow solid; mp: 260-262° C.; R_{f}=0.49 (MeOH:EtOAc, 5:5. v/v); IR (KBr) νₘₐₓ: 3454 cm⁻¹. ¹H NMR (400 MHz, DMSO-d⁶): δ (ppm)=7.05 (D, 7.48 (d, J=4.8 Hz, 1H), 7.32 (d, J=5.4 Hz, 1H), J=6.2 Hz, 3H), 7.61 (d, J=6.1 Hz, 1H), 7.66 (d, J=6.1 Hz, 1H), 7.71 (d, J=6.2 Hz, 1H), 7.78 (d, J=8.2 Hz, 1H), 7.86 (d, J=6.7 Hz, 1H), 8.08 (s, 1H), 8.18 (d, J=6.2 Hz, 1H), 8.35 (s, 1H), 11.43 (s, 1H). ¹³C NMR (100 MHz, DMSO-d⁶): δ (ppm)=113.4, 113.8, 113.9, 116.6, 116.9, 117.2, 120.2, 121.5, 122.4 (d, J=10 Hz), 124.4, 124.7, 124.8, 126.0, 129.9, 130.3, 132.9, 133.5, 139.3, 163.0 (d, J=250 Hz). MS (ESI+): m/z=490.0. ESI-HR-MS calcd for C₂₅H₁₅BrClFN₂[M⁺]: 490.0116, found: 490.0119.

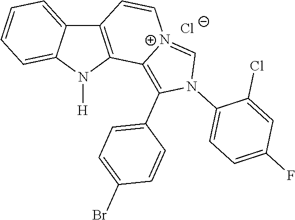 [SMILES: Fc1ccc(-n2c[n+]3ccc4c5ccccc5[nH]c4c3c2-c2ccc(Br)cc2)c(Cl)c1 ; [Cl-]]

1-(4-Bromophenyl)-2-(3,4-difluorophenyl)-2,11-dihydroimidazo[1′,5′:1,2]pyrido[3,4-b]indol-4-ium chloride (8abm). Yield: 68% (0.296 g from 0.3 g); a white solid; mp: 250-252° C.; R_{f}=0.48 (MeOH:EtOAc, 5:5. v/v); IR (KBr) νₘₐₓ: 3387 cm⁻¹. H NMR (400 MHz, DMSO-d⁶): δ (ppm)=7.31 (t, J=7.6 Hz, 1H), 7.41-7.43 (m, 1H), 7.45-7.49 (m, 4H), 7.66 (d, J=8.2 Hz, 1H), 7.71 (m, 1H), 7.78 (d, J=8.2 Hz, 1H), 7.90-7.94 (d, J=6.9 Hz, 1H), 8.19 (d, J=7.0 Hz, 2H), 8.60 (d, J=6.9 Hz, 1H), 10.63 (s, 1H), 11.43 (s, 1H). ¹³C NMR (100 MHz, DMSO-d⁶): δ (ppm)=110.4, 113.5, 114.1, 116.4 (d, J=60 Hz), 117.9, 118.1, 119.0, 119.2, 120.3, 120.5, 122.0 (d, J=90 Hz), 122.6, 124.2, 124.7, 124.9, 125.5, 126.2, 129.8, 130.5, 133.1, 133.6, 139.4, 153.5, 160.2, 163.1. MS (ESI+): m/z=474.0. ESI-HR-MS calcd for C₂₅H₁₅BrF₂N₃[M⁺]: 474.0412, found: 474.0418.

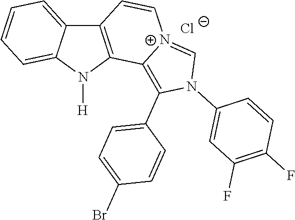 [SMILES: Fc1ccc(-n2c[n+]3ccc4c5ccccc5[nH]c4c3c2-c2ccc(Br)cc2)cc1F ; [Cl-]]

1-(2-Bromophenyl)-2-phenyl-2,11-dihydroimidazo[1′,5′:1,2]pyrido[3,4-b]indol-4-ium chloride (8aca). Yield: 78% (0.316 g from 0.3 g); a brown solid; mp: 273-275° C.; R_{f}=0.43 (MeOH:EtOAc, 5:5. v/v); IR (KBr) νₘₐₓ: 3357 cm⁻¹. ¹H NMR (400 MHz, DMSO-d⁶): δ (ppm)=7.29 (t, J=7.2 Hz, 1H), 7.40 (d, J=7.2 Hz, 1H), 7.51 (t, J=6.0 Hz, 3H), 7.57 (d, J=6.5 Hz, 3H), 7.65 (d, J=7.9 Hz, 1H), 7.79 (d, J=8.4 Hz, 2H), 8.17 (t, J=6.1 Hz, 2H), 8.18 (s, 1H), 8.50 (d, J=7.2 Hz, 2H), 10.48 (s, 1H). ¹³C NMR (100 MHz, DMSO-d⁶): δ (ppm)=113.3, 113.7, 113.8, 116.5, 116.9, 117.2, 120.1, 120.4, 121.3, 122.3, 122.5, 124.4, 124.7, 125.9, 127.2, 129.1, 129.4, 132.9, 133.3, 134.0, 134.1, 139.2. MS (ESI+): m/z=438.0. ESI-HR-MS calcd for C₂₅H₁₇BrN₃[M⁺]: 438.0600, found: 438.0611.

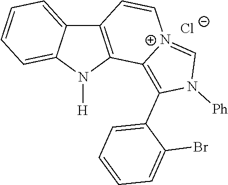 [SMILES: Brc1ccccc1-c1c2c3[nH]c4ccccc4c3cc[n+]2cn1-c1ccccc1 ; [Cl-]]

1-(2-Bromophenyl)-2-(4-bromophenyl)-2,11-dihydroimidazo[1′,5′:1,2]pyrido[3,4-b]indol-4-ium chloride (8acb).Yield: 67% (0.316 g from 0.3 g); a white solid; mp: 252-254° C.; R_{f}=0.45 (MeOH:EtOAc, 5:5. v/v); IR (KBr) νₘₐₓ: 3410 cm⁻¹. ¹H NMR (400 MHz, DMSO-d⁶): δ (ppm)=7.32 (d, J=7.6 Hz, 1H), 7.43 (t, J=7.7 Hz, 1H), 7.49 (d, J=8.6 Hz, 2H), 7.58-7.61 (m, 1H), 7.64 (d, J=8.3 Hz, 1H), 7.70-7.73 (m, 1H), 7.79 (d, J=8.6 Hz, 2H), 7.86-7.88 (m, 1H), 8.20 (d, J=7.2 Hz, 2H), 8.58 (s, 1H), 10.69 (s, 1H), 11.61 (s, 1H). ¹³C NMR (100 MHz, DMSO-d⁶): δ (ppm)=113.5, 114.0, 114.2, 116.7, 120.4, 121.3, 121.5, 122.6, 124.6, 126.1, 126.2, 126.3, 128.9, 129.1, 129.6, 133.1, 133.3, 133.8, 133.9, 135.2, 139.8. MS (ESI+): m/z=515.9. ESI-HR-MS calcd for C₂₅H₁₆Br₂N₃[M⁺]: 515.9705, found: 515.9710.

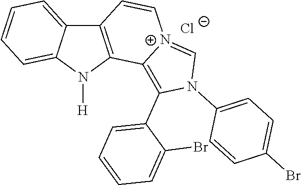 [SMILES: Brc1ccc(-n2c[n+]3ccc4c5ccccc5[nH]c4c3c2-c2ccccc2Br)cc1 ; [Cl-]]

1-(2-Bromophenyl)-2-(4-chlorophenyl)-2,11-dihydrolmidazo[1′,5′:1,2]pyrido[3,4-b]indol-4-ium (8acc). Yield: 78% (0.338 g from 0.3 g); a white solid; mp: 282-284° C.; R_{f}=0.44 (MeOH:EtOAc, 5:5. v/v); IR (KBr) νₘₐₓ: 3390 cm⁻¹. ¹H NMR (400 MHz, DMSO-d⁶): δ (ppm)=7.29 (t, J=7.6 Hz, 1H), 7.41 (t, J=7.1 Hz, 1H), 7.56-7.58 (m, 4H), 7.62-7.66 (m, 3H), 7.70-7.73 (m, 1H), 7.84 (m, 1H), 8.19 (d, J=6.8 Hz, 2H), 8.60 (d, J=7.0 Hz, 1H), 10.78 (s, 1H). ¹³C NMR (100 MHz, DMSO-d⁶): δ (ppm)=113.6, 113.9, 114.1, 116.6, 120.4, 121.4, 121.6, 122.6, 124.8, 126.0, 126.1, 126.3, 128.7, 129.0, 129.6, 130.1, 132.9, 133.8, 135.2, 135.9, 139.9. MS (ESI+): m/z=472.0. ESI-HR-MS calcd for C₂₅H₁₆BrClN₃[M⁺]: 472.0211, found: 472.0208.

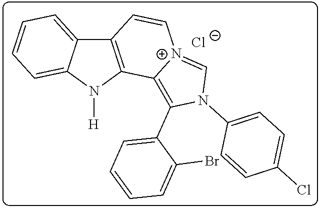 [SMILES: Clc1ccc(-n2c[n+]3ccc4c5ccccc5[nH]c4c3c2-c2ccccc2Br)cc1 ; [Cl-]]

1-(2-Bromophenyl)-2-(4-fluorophenyl)-2,11-dihydroimidazo[1′,5′:1,2]pyrido[3,4-b]indol-4-ium chloride (8acd). Yield: 70% (0.294 g from 0.3 g); a light green solid; mp: 267-269° C.; =0.43 (MeOH:EtOAc, 5:5. v/v); IR (KBr) νₘₐₓ: 3310 cm⁻¹. ¹H NMR (400 MHz, DMSO-d⁶): δ (ppm)=7.33 (d, J=6.6 Hz, 1H), 7.44 (s, 3H), 7.59-7.64 (m, 5H), 7.72 (s, 1H), 7.87 (s, 1H), 8.21 (d, J=5.7 Hz, 2H), 8.64 (d, J=5.7 Hz, 1H), 10.77 (s, 1H), 11.59 (s, 1H). ¹³C NMR (100 MHz, DMSO-d⁶): δ (ppm)=113.5, 114.0, 114.1, 116.8, 117.0, 117.3, 120.4, 121.4, 121.5 (d, J=10 Hz), 122.6, 124.6, 126.1, 126.2, 126.3, 129.1, 129.5 (d, J=10 Hz), 129.7, 130.4, 133.8, 135.3, 139.6, 163.1 (d, J=10 Hz). MS (ESI+): m/z=456.0. ESI-HR-MS calcd for C₂₅H₁₆BrFN₃[M⁺]: 456.0506, found: 456.0501.

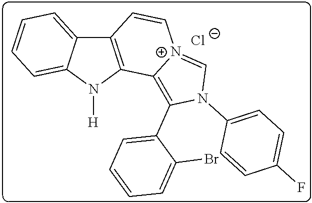 [SMILES: Fc1ccc(-n2c[n+]3ccc4c5ccccc5[nH]c4c3c2-c2ccccc2Br)cc1 ; [Cl-]]

1-(2-Bromophenyl)-2-(3-chlorophenyl)-2,11-dihydroimidazo[1′,5′:1,2]pyrido[3,4-b]indol-4-ium chloride (8acf). Yield: 76% (0.329 g from 0.3 g); a brown solid; mp: 259-261° C.; R_{f}=0.46 MeH:EtOAc, 5:5. v/v); IR (KBr) νₘₐₓ: 3361 cm⁻¹. ¹H NMR (400 MHz, DMSO-d⁶): δ (ppm)=7.32 (t, J=7.7 Hz, 1H), 7.41-7.46 (m, 3H), 7.52 (d, J=8.6 Hz, 2H), 7.57-7.60 (m, 2H), 7.65 (d, J=8.2 Hz, 1H), 7.81 (d, J=8.5 Hz, 2H), 8.18 (d, J=6.3 Hz, 2H), 8.56 (d, J=7.0 Hz, 1H), 10.57 (s, 1H), 11.35 (s, 1H). ¹³C NMR (100 MHz, DMSO-d⁶): δ (ppm)=113.5, 114.0, 116.6, 117.2, 117.4, 120.3, 120.6, 121.6, 122.6, 124.6, 124.9, 126.1, 129.6, 133.1, 133.5, 134.2, 134.3, 139.4. MS (ESI+): m/z=472.0. ESI-HR-MS calcd for C₂₅H₁₆BrClN₃[M⁺]: 472.0211, found: 472.0215.

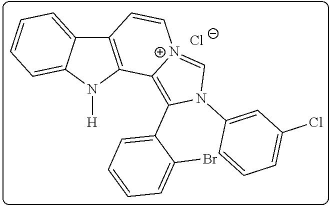 [SMILES: Clc1cccc(-n2c[n+]3ccc4c5ccccc5[nH]c4c3c2-c2ccccc2Br)c1 ; [Cl-]]

1-(2-Bromophenyl)-2-(3-fluorophenyl)-2,11-dihydroimidazo[1′,5′:1,2]pyrido[3,4-b]indol-4-ium (8aci). Yield: 75% (0.293 g from 0.3 g); a light brown solid; mp: 280-282° C.; R_{f}=0.45 (MeOH:EtOAc, 5:5. v/v); IR (KBr) νₘₐₓ: 3360 cm⁻¹. ¹H NMR (400 MHz, DMSO-d⁶): δ (ppm)=7.29 (m, 1H), 7.39-7.47 (m, 5H), 7.57-7.60 (m, 2H), 7.63-7.67 (m, 3H), 8.18 (t, J=6.5 Hz, 2H), 8.56 (d, J=7.2 Hz, 1H), 10.58 (s, 1H), 11.36 (s, 1H). ¹³C NMR (100 MHz, DMSO-d⁶_): δ (ppm)=113.5, 113.9, 114.0, 116.7, 117.2 (d, J=30 Hz), 118.6, 120.3, 120.6, 121.6, 122.5, 122.7, 123.6, 124.1, 124.0 (d, J=30 Hz), 126.1, 127.7, 129.0, 129.6, 129.9, 130.0, 130.1, 133.4, 133.9, 135.8, 139.4, 140.7. MS (ESI+): m/z=456.0. ESI-HR-MS calcd for C₂₅H₁₆BrFN₃[M⁺]: 456.0506, found: 456.0512.

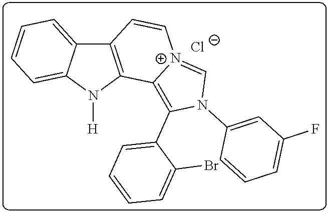 [SMILES: Fc1cccc(-n2c[n+]3ccc4c5ccccc5[nH]c4c3c2-c2ccccc2Br)c1 ; [Cl-]]

2-(4-Bromophenyl)-1-(4-chlorophenyl)-2,11-dihydroimidazo[1′,5′:1,2]pyrido[3,4-b]indol-4-ium chloride (8adb). Yield: 69% (0.342 g from 0.3 g); a white solid; mp: 278-281° C.; R_{f}=0.44 (MeOH:EtOAc, 5:5. v/v); IR (KBr) νₘₐₓ: 3402 cm⁻¹. ¹H NMR (400 MHz, DMSO-d⁶): δ (ppm)=7.26-7.30 (m, 1H), 7.38-7.42 (m, 1H), 7.52-7.56 (m, 4H), 7.64 (d, J=8.3 Hz, 3H), 7.82 (d, J=8.4 Hz, 2H), 8.15-8.19 (m, 2H), 8.49 (d, J=4.1 Hz, 1H), 10.52 (s, 1H). ¹³C NMR (100 MHz, DMSO-d⁶): δ (ppm)=113.4, 114.1, 116.8, 119.6, 120.4, 121.5, 121.6, 122.6, 124.5, 124.6, 126.2, 128.7, 128.9, 129.9, 130.8, 133.1, 133.4, 133.7, 135.0, 135.6, 139.6. MS (ESI+): m/z=472.0. ESI-HR-MS calcd for C₂₅H₁₆BrClN₃[M⁺]: 472.0211, found: 472.0217.

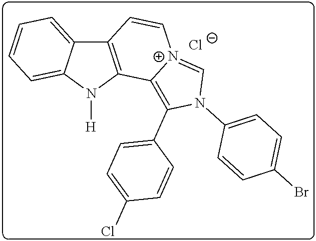 [SMILES: Clc1ccc(-c2c3c4[nH]c5ccccc5c4cc[n+]3cn2-c2ccc(Br)cc2)cc1 ; [Cl-]]

1,2-Bis(4-chlorophenyl)-2,11-dihydrolmidazo[1′,5′:1,2]pyrido[3,4-b]indol-4-ium chloride (8adc). Yield: 83% (0.348 g from 0.3 g); a white solid; mp: 269-271° C.; R_{f}=0.45 (MeOH:EtOAc, 5:5. v/v); IR (KBr) νₘₐₓ: 3386 cm⁻¹. ¹H NMR (400 MHz, DMSO-d⁶): δ (ppm)=7.28-7.31 (m, 1H), 7.40-7.43 (m, 1H), 7.54 (d, J=6.5 Hz, 2H), 7.60-7.62 (m, 4H), 7.64-7.68 (m, 3H), 8.16-8.18 (m, 2H), 8.58 (d, J=5.4 Hz, 1H), 10.68 (s, 1H). ¹³C NMR (100 MHz, DMSO-d⁶): δ (ppm)=113.6, 114.0, 114.1, 116.7, 120.3, 120.7, 121.5, a 122.2, 122.7, 124.1, 124.9, 126.1, 128.8, 129.2, 129.4, 129.5, 130.2, 130.5, 133.0, 133.4, 135.9, 136.0, 139.5. MS (ESI+): m/z=428.0. ESI-HR-MS calcd for C₂₅H₁₆Cl₂N₃[M⁺]: 428.0716, found: 428.0717.

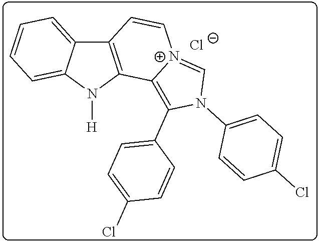 [SMILES: Clc1ccc(-c2c3c4[nH]c5ccccc5c4cc[n+]3cn2-c2ccc(Cl)cc2)cc1 ; [Cl-]]

1-(4-Chlorophenyl)-2-(4-fluorophenyl)-2,11-dihydroimidazo[1′,5′:1,2]pyrido[3,4-b]indol-4-ium chloride (8add). Yield: 86% (0.376 g from 0.3 g); a light yellow solid; mp: 230-232° C.; R_{f}=0.47 (MeOH:EtOAc, 5:5. v/v); IR (KBr) νₘₐₓ: 3400 cm⁻¹. ¹H NMR (400 MHz, DMSO-d⁶): δ (ppm)=7.32 (t, J=7.4 Hz, 1H), 7.41-7.48 (m, 3H), 7.54 (d, J=8.2 Hz, 2H), 7.63-7.68 (m, 5H), 8.18-8.21 (m, 2H), 8.60 (d, J=7.1 Hz, 1H), 10.68 (s, 1H), 11.43 (brs, 1H). ¹³C NMR (100 MHz, DMSO-d⁶): δ (ppm)=113.5, 113.9, 114.0, 116.7, 117.1, 117.2, 120.3, 121.5, 122.4, 122.6, 124.1, 124.8, 125.0, 126.1, 129.7, 130.0, 130.1, 130.2, 130.4, 133.4, 135.9, 139.4, 162.2. MS (ESI+): m/z=412.1. ESI-HR-MS calcd for C₂₅H₁₆ClFN₃[M⁺+H]: 412.1011, found: 412.1018.

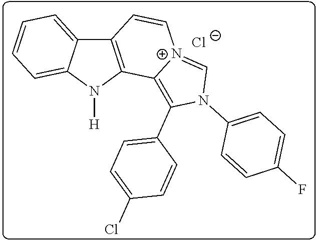 [SMILES: Fc1ccc(-n2c[n+]3ccc4c5ccccc5[nH]c4c3c2-c2ccc(Cl)cc2)cc1 ; [Cl-]]

2-(3-Bromophenyl)-1-(4-chlorophenyl)-2,11-dihydroimidazo[1′,5′:1,2]pyrido[3,4-b]indol-4-ium chloride (8ade). Yield: 76% (0.377 g from 0.3 g); a white solid; mp: 301-303° C.; R_{f}=0.44 (MeOH:EtOAc, 5:5. v/v); IR (KBr) νₘₐₓ: 3402 cm⁻¹. ¹H NMR (400 MHz, DMSO-d⁶): δ (ppm)=7.32 (t, J=7.1 Hz, 1H), 7.44 (t, J=6.7 Hz, 1H), 7.56 (d, J=8.3 Hz, 3H), 7.57-7.59 (m, 4H), 7.94-7.98 (m, 1H), 8.20 (d, J=6.8 Hz, 2H), 8.60 (s, 1H), 10.69 (s, 1H), 11.44 (s, 1H). ¹³C NMR (100 MHz, DMSO-d⁶): δ (ppm)=113.6, 114.1, 116.7, 120.3, 120.6, 121.6, 122.2, 122.7, 126.1, 126.8, 129.7, 130.2, 133.5, 135.2, 135.9, 139.4, 141.1. MS (ESI+): m/z=472.0. ESI-HR-MS calcd for C₂₅H₁₆BrClN₃[M⁺]: 472.0211, found: 472.0213.

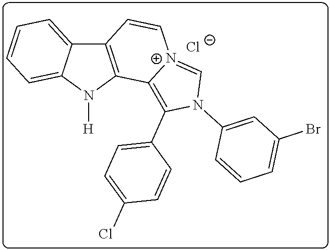 [SMILES: Clc1ccc(-c2c3c4[nH]c5ccccc5c4cc[n+]3cn2-c2cccc(Br)c2)cc1 ; [Cl-]]

2-(3-Chlorophenyl)-1-(4-chlorophenyl)-2,11-dihydroimidazo[1′,5′:1,2]pyrido[3,4-b]indol-4-ium chloride (8adf). Yield: 78% (0.354 g from 0.3 g); brown solid; mp: 299-301° C.; R_{f}=0.39 (MeOH:EtOAc, 5:5. v/v); IR (KBr) νₘₐₓ: 3413 cm⁻¹. ¹H NMR (400 MHz, DMSO-d⁶): δ (ppm)=7.29 (d, J=7.3 Hz, 1H), 7.40 (t, J=7.4 Hz, 1H), 7.49-7.54 (m, 4H), 7.57-7.58 (m, 3H), 7.66 (t, J=7.9 Hz, 1H), 7.76 (s, 1H), 8.17 (d, J=7.0 Hz, 2H), 8.54 (t, J=6.9 Hz, 1H), 10.63 (s, 1H). ¹³C NMR (100 MHz, DMSO-d⁶): δ (ppm)=113.7, 113.9, 114.0, 116.6, 120.2, 120.4, 121.5, 122.7, 123.4, 125.1, 126.0, 126.4, 127.6, 129.5, 129.9, 130.9, 131.1, 131.6, 133.9, 135.4, 139.5. MS (ESI+): m/z=428.0. ESI-HR-MS calcd for C₂₅H₁₆Cl₂N₃[M⁺]: 428.0716, found: 428.0718.

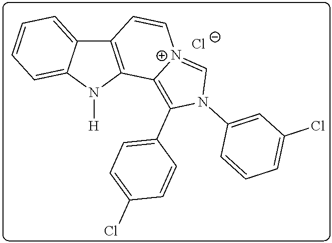 [SMILES: Clc1ccc(-c2c3c4[nH]c5ccccc5c4cc[n+]3cn2-c2cccc(Cl)c2)cc1 ; [Cl-]]

1-(4-Chlorophenyl)-2-(4-methoxyphenyl)-2,11-dihydroimidazo[1′,5′:1,2]pyrido[3,4-b]indol-4-ium chloride (8adg). Yield: 68% (0.306 g from 0.3 g); a light yellow solid; mp: 260-262° C.; R_{f}=0.39 (MeOH:EtOAc, 5:5. v/v); IR (KBr) νₘₐₓ: 3389 cm⁻¹. ¹H NMR (400 MHz, DMSO-d⁶): δ (ppm)=3.20 (S, 1H), 7.09-7.11 (m, 2H), 7.31 (t, J=7.2 Hz, 1H), 7.40-7.44 (m, 1H), 7.48-7.55 (m, 4H), 7.62-7.67 (m, 3H), 8.17 (t, J=8.7 Hz, 2H), 8.59 (d, J=7.2 Hz, 1H), 10.62 (s, 1H). ¹³C NMR (100 MHz, DMSO-d⁶): δ (ppm)=57.8, 113.8, 113.9, 114.0, 116.6, 120.2, 120.4, 121.4, 122.7, 123.4, 125.1, 125.9, 126.4, 127.6, 129.6, 129.9, 130.9, 131.1, 131.5, 131.6, 133.9, 135.5, 139.6, 167.0. MS (ESI+): m/z=424.1. ESI-HR-MS calcd for C₂₆H₁₉ClN₃O [M⁺]: 424.1211, found: 424.1208.

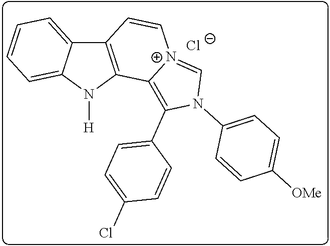 [SMILES: COc1ccc(-n2c[n+]3ccc4c5ccccc5[nH]c4c3c2-c2ccc(Cl)cc2)cc1 ; [Cl-]]

1-(2-Chlorophenyl)-2-phenyl-2,11-dihydroimidazo[1′,5′:1,2]pyrido[3,4-b]indol-4-ium chloride (8aea). Yield: 68% (0.286 g from 0.3 g); a white solid; mp: 298-300° C.; R_{f}=0.45 MeOH:EtOAc 5:5. v/v); IR (KBr) νₘₐₓ: 3380 cm⁻¹. ¹H NMR (400 MHz, DMSO-d⁶): δ (ppm)=7.30 (t, J=7.4 Hz, 1H), 7.41 (t, J=7.1 Hz, 1H), 7.49-7.53 (m, 4H), 7.55-7.60 (m, 3H), 7.65 (t, J=7.4 Hz, 1H), 7.76 (d, J=8.4 Hz, 2H), 8.15-8.18 (m, 2H), 8.53 (d, J=8.2 Hz, 2H), 10.59 (s, 1H). ¹³C NMR (100 MHz, DMSO-d⁶): δ (ppm)=113.7, 113.9, 114.7, 120.2, 121.5, 122.7, 123.5, 124.5, 125.1, 126.0, 129.5, 129.9, 130.9, 131.5, 133.0, 133.6, 139.4. MS (ESI+): m/z=394.1. ESI-HR-MS calcd for C₂₅H₁₇ClN₃[M⁺]: 394.1106, found: 394.1113.

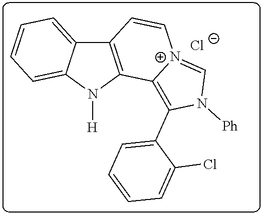 [SMILES: Clc1ccccc1-c1c2c3[nH]c4ccccc4c3cc[n+]2cn1-c1ccccc1 ; [Cl-]]

2-(4-Bromophenyl)-1-(2-chlorophenyl)-2,11-dihydroimidazo[1′,5′:1,2]pyrido[3,4-b]indol-4-ium chloride (8aeb). Yield: 69% (0.342 g from 0.3 g); a light yellow solid; mp: 230-232° C.; R_{f}=0.44 (Me H:EtOAc, 5:5. v/v); IR (KBr) νₘₐₓ: 3401 cm⁻¹. ¹H NMR (400 MHz, DMSO-d⁶): δ (ppm)=7.33 (t, J=7.5 Hz, 1H), 7.44 (t, J=7.4 Hz, 1H), 7.48-7.50 (m, 2H), 7.56 (t, J=7.3 Hz, 1H), 7.64 (d, J=8.3 Hz, 1H), 7.69-7.73 (m, 3H), 7.79 (d, J=8.2 Hz, 2H), 8.21 (d, J=7.3 Hz, 2H), 8.61 (d, J=7.0 Hz, 1H), 10.75 (s, 1H), 11.60 (s, 1H). ¹³C NMR (100 MHz, DMSO-d⁶): δ (ppm)=113.4, 114.1, 116.8, 119.6, 120.4, 121.6, 121.7, 122.6, 124.0, 124.5, 124.6, 126.2, 128.7, 128.9, 129.9, 130.8, 133.1, 133.4, 133.7, 135.0, 135.5, 139.6. MS (ESI+): m/z=472.0. ESI-HR-MS calcd for C₂₅H₁₆BrClN₃[M⁺]: 472.0211, found: 472.0208.

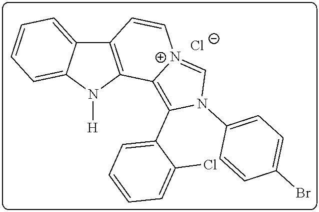 [SMILES: Clc1ccccc1-c1c2c3[nH]c4ccccc4c3cc[n+]2cn1-c1ccc(Br)cc1 ; [Cl-]]

1-(2-Chlorophenyl)-2-(4-chlorophenyl)-2,11-dihydroimidazo[1′,5′:1,2]pyrido[3,4-b]indol-4-ium chloride (8aec). Yield: 81% (0.367 g from 0.3 g); a white solid; mp: 284-286° C.; R_{f}=0.42 (MeOH:EtOAc, 5:5. v/v); IR (KBr) νₘₐₓ: 3391 cm⁻¹. ¹H NMR (400 MHz, DMSO-d⁶): δ (ppm)=7.31 (t, J=7.7 Hz, 1H), 7.43 (t, J=8.0 Hz, 1H), 7.53-7.58 (m, 3H), 7.64-7.68 (m, 4H), 7.70-7.73 (m, 2H), 8.20 (t, J=7.1 Hz, 2H), 8.65 (d, J=7.2 Hz, 1H), 10.85 (s, 1H), 11.65 (s, 1H). ¹³C NMR (100 MHz, DMSO-d⁶): δ (ppm)=113.5, 114.1, 116.8, 119.7, 120.4, 121.5, 121.6, 122.6, 124.0, 124.5, 126.2, 128.7, 130.0, 130.2, 130.8, 132.9, 133.7, 135.0, 135.6, 135.9, 139.6. MS (ESI+): m/z=428.0. ESI-HR-MS calcd for C₂₅H₁₆Cl₂N₃[M⁺]: 428.0716, found: 428.0718.

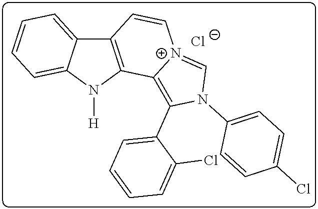 [SMILES: Clc1ccc(-n2c[n+]3ccc4c5ccccc5[nH]c4c3c2-c2ccccc2Cl)cc1 ; [Cl-]]

1-(2-Chlorophenyl)-2-(4-fluorophenyl)-2,11-dihydroimidazo[1′,5′:1,2]pyrido[3,4-b]indol-4-ium chloride (8aed). Yield: 80% (0.250 g from 0.3 g); a white solid; mp: 267-269° C.; R_{f}=0.46 (MeOH:EtOAc, 5:5. v/v); IR (KBr) νᵥₐₓ: 3367 cm⁻¹. ¹H NMR (400 MHz, DMSO-d⁶): δ (ppm)=7.33 (t, J=7.7 Hz, 1H), 7.42-7.46 (m, 3H), 7.54-7.57 (m, 1H), 7.59-7.64 (m, 3H), 7.65-7.68 (m, 1H), 7.69-7.72 (m, 2H), 8.21 (d, J=7.1 Hz, 2H), 8.57 (d, J=7.2 Hz, 1H), 10.62 (s, 1H), 11.57 (s, 1H). ¹³C NMR (100 MHz, DMSO-d⁶): δ (ppm)=113.4, 114.1, 116.8, 117.2 (d, J=20 Hz), 119.9, 120.4, 121.6 (d, J=10 Hz), 122.6, 124.1, 124.6, 126.2, 128.6, 129.3, 129.4, 130.0, 130.4, 130.8, 133.7, 135.0, 135.6, 139.6, 163.2 (d, J=260 Hz). MS (ESI+): m/z=412.1. ESI-HR-MS calcd for C₂₅H₁₆ClFN₃[M⁺]: 412.1011, found: 412.1017.

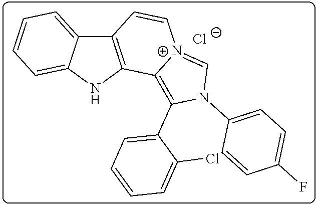 [SMILES: Fc1ccc(-n2c[n+]3ccc4c5ccccc5[nH]c4c3c2-c2ccccc2Cl)cc1 ; [Cl-]]

1-(3-Chlorophenyl)-2-(4-chlorophenyl)-2,11-dihydrolmidazo[1′,5′:1,2]pyrido[3,4-b]indol-4-ium chloride (8afc). Yield: 79% (0.358 g from 0.3 g); a white solid; mp: 290-292° C.; R_{f}=0.42 (MeOH:EtOAc, 5:5. v/v); IR (KBr) νₘₐₓ: 3360 cm⁻¹. ¹H NMR (400 MHz, DMSO-d⁶): δ (ppm)=7.31 (t, J=7.3 Hz, 1H), 7.41-7.48 (m, 2H), 7.57-7.63 (m, 5H), 7.68 (t, J=8.6 Hz, 3H), 8.19 (d, J=6.3 Hz, 2H), 8.55 (d, J=6.9 Hz, 1H), 10.57 (s, 1H), 11.51 (s, 1H). ¹³C NMR (100 MHz, DMSO-d⁶): δ (ppm)=113.6, 114.1, 120.3, 121.5, 122.0, 122.7, 126.2, 127.1, 129.4, 129.5, 130.2, 130.5, 131.1, 131.4, 131.7, 132.9, 134.3, 136.0. MS (ESI+): m/z=428.0. ESI-HR-MS calcd for C₂₅H₁₆N₂O₃[M⁺]: 428.0716, found: 428.0716.

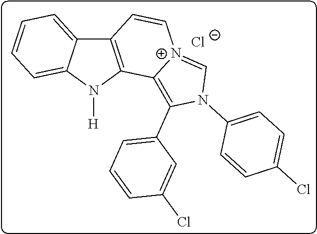 [SMILES: Clc1ccc(-n2c[n+]3ccc4c5ccccc5[nH]c4c3c2-c2cccc(Cl)c2)cc1 ; [Cl-]]

1-(3-Chlorophenyl)-2-(4-fluorophenyl)-2,11-dihydroimidazo[1′,5′:1,2]pyrido[3,4-b]indol-4-ium chloride (8afd). Yield: 78% (0.341 g from 0.3 g); a white solid; mp: 293-295° C.; R_{f}=0.44 (MeOH:EtOAc, 5:5. v/v); IR (KBr) νₘₐₓ: 3350 cm⁻¹. ¹H NMR (400 MHz, DMSO-d⁶): δ (ppm)=7.31 (t, J=7.1 Hz, 1H), 7.41-7.48 (m, 1H), 7.53-7.58 (m, 1H), 7.60 (s, 1H), 7.64-7.66 (m, 5H), 7.81 (d, J=8.3 Hz, 2H), 8.19 (d, J=7.1 Hz, 2H), 8.61 (d, J=6.8 Hz, 1H), 10.68 (s, 1H), 11.53 (s, 1H). ¹³C NMR (100 MHz, DMSO-d⁶): δ (ppm)=113.5, 113.8, 113.9, 116.7, 117.1, 117.4, 120.3, 120.6, 121.5, 122.5, 122.6, 124.5, 124.9, 126.1, 129.3, 129.6, 133.1, 133.5, 134.2, 134.3, 139.4, 163.8 (d, J=240 Hz). MS (ESI+): m/z=412.1. ESI-HR-MS calcd for C₂₅H₁₆ClFN₃[M⁺]: 412.1011, found: 412.1016.

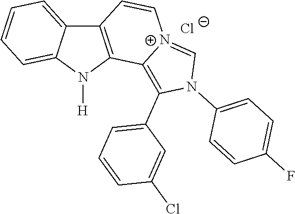 [SMILES: Fc1ccc(-n2c[n+]3ccc4c5ccccc5[nH]c4c3c2-c2cccc(Cl)c2)cc1 ; [Cl-]]

1-(4-Fluorophenyl)-2-phenyl-2,11-dihydroimidazo[1′,5′:1,2]pyrido[3,4-b]indol-4-ium chloride (8aga). Yield: 63% (0.269 g from 0.3 g); a light yellow solid; mp: 277-279° C.; R_{f}=0.47 (MeOH:EtOAc, 5:5. v/v); IR (KBr) νₘₐₓ: 3368 cm⁻¹. ¹H NMR (400 MHz, DMSO-d⁶): δ (ppm)=7.32 (t, J=8.2 Hz, 2H), 7.41 (t, J=6.8 Hz, 4H), 7.49-7.53 (m, 1H), 7.57-7.60 (m, 4H), 7.65 (t, J=8.1 Hz, 1H), 8.17 (t, J=8.4 Hz, 2H), 8.52 (d, J=6.8 Hz, 1H), 10.49 (d, J=7.8 Hz, 1H), 11.32 (s, 1H). ¹³C NMR (100 MHz, DMSO-d⁶): δ (ppm)=113.5, 113.8, 116.7, 117.0, 117.2, 120.2, 120.6, 121.5, 121.7, 122.5, 122.6, 122.7, 124.9, 126.0, 127.2, 124.4, 128.6, 129.2, 130.1, 131.1, 134.1, 134.2. MS (ESI+): m/z=378.1. ESI-HR-MS calcd for C₂₅H₁₇FN₃[M⁺]: 378.1401, found: 378.1406.

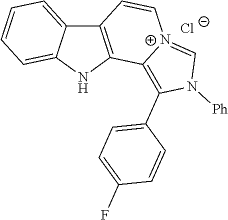 [SMILES: Fc1ccc(-c2c3c4[nH]c5ccccc5c4cc[n+]3cn2-c2ccccc2)cc1 ; [Cl-]]

2-(4-Bromophenyl)-1-(4-fluorophenyl)-2,11-dihydroimidazo[1′,5′:1,2]pyrido[3,4-b]indol-4-ium chloride (8agb). Yield: 64% (0.325 g from 0.3 g); a white solid; mp 283-285° C.; R_{f}=0.46 (MeOH:EtOAc, 5:5. v/v); IR (KBr) νₘₐₓ: 3401 cm⁻¹. ¹H NMR (400 MHz, DMSO-d⁶): δ (ppm)=7.32 (d, J=7.3 Hz, 1H), 7.43 (t, J=8.5 Hz, 3H), 7.53 (d, J=8.3 Hz, 2H), 7.59 (t, J=7.5 Hz, 2H), 7.66 (d, J=7.9 Hz, 1H), 7.81 (d, J=8.2 Hz, 2H), 8.20 (d, J=5.0 Hz, 2H), 8.60 (d, J=6.9 Hz, 1H), 10.68 (s, 1H), 11.37 (s, 1H). ¹³C NMR (100 MHz, DMSO-d⁶): δ (ppm)=113.5, 113.8, 114.0, 116.7, 117.1, 117.4, 120.3, 120.6, 121.5, 122.5, 122.7, 124.6, 124.9, 126.1, 129.3, 129.6, 133.1, 133.5, 134.2, 134.3, 139.4, 163.8 (d, J=250 Hz). MS (ESI+): m/z=456.0. ESI-HR-MS calcd for C₂₅H₁₆BrFN₃[M⁺]: 456.0506, found: 456.0501.

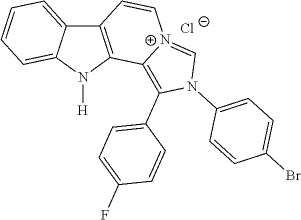 [SMILES: Fc1ccc(-c2c3c4[nH]c5ccccc5c4cc[n+]3cn2-c2ccc(Br)cc2)cc1 ; [Cl-]]

2-(4-Chlorophenyl)-1-(4-fluorophenyl)-2,11-dihydrolmidazo[1′,5′:1,2]pyrido[3,4-b]indol-4-ium chloride (8agc). Yield: 75% (0.346 g from 0.3 g); a white solid; mp: 298-300° C.; R_{f}=0.44 (MeOH:EtOAc, 5:5. v/v); IR (KBr) νₘₐₓ: 3372 cm⁻¹. ¹H NMR (400 MHz, DMSO-d⁶): δ (ppm)=7.31 (d, J=7.3 Hz, 1H), 7.42 (t, J=6.7 Hz, 2H), 7.60-7.66 (m, 8H), 8.18 (d, J=7.2 Hz, 2H), 8.61 (d, J=6.0 Hz, 1H), 10.71 (s, 1H), 11.37 (s, 1H). ¹³C NMR (100 MHz, DMSO-d⁶): δ (ppm)=113.6, 114.0, 114.1, 116.8, 117.2, 117.4, 120.4, 120.7, 121.6, 122.6, 122.7, 124.9, 126.1, 129.4, 129.5, 130.2, 130.6, 133.1, 134.3, 134.4, 135.9, 139.4, 163.8 (d, J=300 Hz). MS (ESI+): m/z=412.1. ESI-HR-MS calcd for C₂₅H₁₆ClFN₃[M⁺]: 412.1011, found: 412.1007.

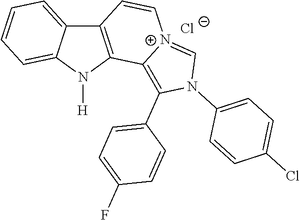 [SMILES: Fc1ccc(-c2c3c4[nH]c5ccccc5c4cc[n+]3cn2-c2ccc(Cl)cc2)cc1 ; [Cl-]]

1,2-Bis(4-fluorophenyl)-2,11-dihydroimidazo[1′,5′:1,2]pyrido[3,4-b]indol-4-ium chloride (8agd). Yield: 79% (0.324 g from 0.3 g); a white solid; mp: 290-292° C.; R_{f}=0.43 (MeOH:EtOAc, 5:5. v/v); IR (KBr) νₘₐₓ: 3412 cm₋₁. ¹H NMR (400 MHz, DMSO-d⁶): δ (ppm)=7.32 (d, J=7.7 Hz, 1H), 7.38-7.48 (m, 5H), 7.57-7.61 (m, 2H), 7.65-7.67 (m, 3H), 8.18 (t, J=6.2 Hz, 2H), 8.57 (d, J=7.2 Hz, 1H), 10.60 (s, 1H). ¹³C NMR (100 MHz, DMSO-d⁶): δ (ppm)=113.6, 113.8, 113.9, 116.6, 117.0, 117.1, 117.2, 117.3, 120.2, 120.6, 121.5, 121.6, 122.7, 122.8, 125.1, 126.0, 129.4, 129.9, 130.1, 130.5, 134.2, 134.3, 139.5, 162.2 (d, J=40 Hz), 164.7 (d, J=60 Hz). MS (ESI+): m/z=396.1. ESI-HR-MS calcd for C₂₅H₁₆F₂N₃[M⁺]: 396.1307, found: 396.1306.

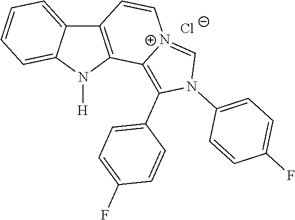 [SMILES: Fc1ccc(-c2c3c4[nH]c5ccccc5c4cc[n+]3cn2-c2ccc(F)cc2)cc1 ; [Cl-]]

2-(3-Bromophenyl)-1-(4-fluorophenyl)-2,11-dihydroimidazo[1′,5′:1,2]pyrido[3,4-b]indol-4-ium chloride (Sage). Yield: 69% (0.350 g from 0.3 g); a light brown solid; mp: 274-276° C.; R_{f}=0.49 (MeOH:EtOAc, 5:5. v/v); IR (KBr) νₘₐₓ: 3394 cm⁻¹. ¹H NMR (400 MHz, DMSO-d⁶): δ (ppm)=7.31 (t, J=7.2 Hz, 1H), 7.39-7.47 (m, 6H), 7.57-7.60 (m, 1H), 7.63-7.67 (m, 3H), 8.18 (t, J=6.5 Hz, 2H), 8.55 (d, J=7.2 Hz, 1H), 10.56 (s, 1H), 10.38 (s, 1H). ¹³C NMR (100 MHz, DMSO-d⁶): δ (ppm)=113.5, 113.8, 114.0, 114.6, 115.4, 116.7, 117.3 (d, J=30 Hz), 119.2, 119.5 (d, J=10 Hz), 120.3, 120.6, 121.5, 122.6 (d, J=20 Hz), 123.7, 123.9, 124.6, 124.9, 126.1, 129.3, 129.6, 133.1, 133.5, 134.2, 134.3, 139.4. MS (ESI+): m/z=456.0 ESI-HR-MS calcd for C₂₅H₁₆BrFN₃[M⁺]: 456.0506, found: 456.0509.

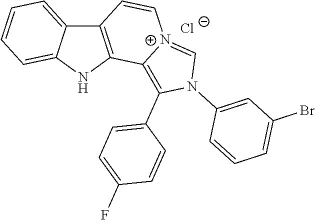 [SMILES: Fc1ccc(-c2c3c4[nH]c5ccccc5c4cc[n+]3cn2-c2cccc(Br)c2)cc1 ; [Cl-]]

2-(3-Chlorophenyl)-1-(4-fluorophenyl)-2,11-dihydroimidazo[1′,5′:1,2]pyrido[3,4-b]indol-4-ium chloride (8agf). Yield: 68% (0.314 g from 0.3 g); a light yellow solid; mp: 281-283° C.; R_{f}=0.45 (MeOH:EtOAc, 5:5. v/v); IR (KBr) νₘₐₓ: 3360 cm⁻¹. ¹H NMR (400 MHz, DMSO-d⁶): δ (ppm)=7.31 (t, J=7.7 Hz, 1H), ac 7.43 (t, J=8.8 Hz, 3H), 7.59-7.62 (m, 4H), 7.67 (t, J=8.8 Hz, 3H), 8.16-8.19 (m, 2H), 8.56 (d, J=7.2 Hz, 1H), 10.60 (s, 1H). ¹³C NMR (100 MHz, DMSO-d⁶): δ (ppm)=113.5, 113.9, 114.0, 116.7, 117.1, 117.3, 120.2, 120.7, 121.5, 122.6, 122.7, 124.9, 126.1, 129.3, 129.4, 130.1, 133.0, 134.2, 134.3, 135.9, 139.4, 162.8. MS (ESI+): m/z=412.1. ESI-HR-MS calcd for C₂₅H₁₆ClFN₃[M⁺]: 412.1011, found: 412.1018.

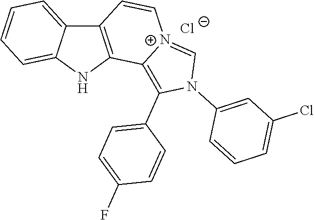 [SMILES: Fc1ccc(-c2c3c4[nH]c5ccccc5c4cc[n+]3cn2-c2cccc(Cl)c2)cc1 ; [Cl-]]

1-(4-Bromophenyl)-2-(4-chlorophenyl)-2,11-dihydroimidazo[1′,5′:1,2]pyrido[3,4-b]indol-4-ium (8ahc). Yield: 84% (0.364 g from 0.3 g); a light yellow solid; mp: 288-290° C.; R_{f}=0.44 (MeOH:EtOAc, 5:5. v/v); IR (KBr) νₘₐₓ: 3378 cm⁻¹. ¹H NMR (400 MHz, DMSO-d⁶): δ (ppm)=7.32 (t, J=7.5 Hz, 1H), 7.42-7.48 (m, 2H), 7.53-7.54 (m, 1H), 7.63 (t, J=7.2 Hz, 2H), 7.67-7.72 (m, 2H), 7.77-7.84 (m, 2H), 7.94 (s, 1H), 8.18-8.21 (m, 2H), 8.60 (d, J=7.2 Hz, 1H), 10.67 (s, 1H), 11.51 (s, 1H). ¹³C NMR (100 MHz, DMSO-d⁶): δ (ppm)=113.5, 114.1, 114.2, 116.7, 120.3, 120.7, 121.6, 121.9, 122.2, 122.7, 122.9, 124.8, 126.2, 127.4, 128.2, 129.4, 129.6, 130.2, 130.8, 131.3, 132.0, 132.1, 133.0, 134.0, 134.2, 135.8, 136.0, 137.2, 139.4. MS (ESI+): m/z=472.0. ESI-HR-MS calcd for C₂₅H₁₆BrClN₃[M⁺]: 472.0211, found: 472.0219.

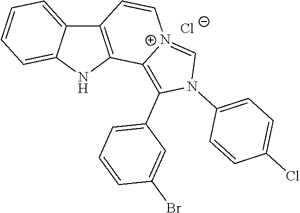 [SMILES: Clc1ccc(-n2c[n+]3ccc4c5ccccc5[nH]c4c3c2-c2cccc(Br)c2)cc1 ; [Cl-]]

2-(4-Chlorophenyl)-1-(3-fluorophenyl)-2,11-dihydrolmidazo[1′,5′:1,2]pyrido[3,4-b]indol-4-ium chloride (8aic). Yield: 76% (0.351 g from 0.3 g); a light brown solid; mp: 281-283° C.; R_{f}=0.46 (MeOH:EtOAc, 5:5. v/v); IR (KBr) νₘₐₓ: 3348 cm⁻¹. ¹H NMR (400 MHz, DMSO-d⁶): δ (ppm)=7.29 (t, J=7.4 Hz, 2H), 7.38-7.46 (m, 3H), 7.57 (t, J=6.3 Hz, 2H), 7.62-7.67 (m, 4H), 8.16 (d, J=7.2 Hz, 2H), 8.56 (d, J=7.1 Hz, 1H), 10.65 (s, 1H). ¹³C NMR (100 MHz, DMSO-d⁶): δ (ppm)=113.5, 113.8, 113.9, 116.7, 117.1, 117.4, 120.3, 120.6, 121.5, 122.5, 122.6, 124.5, 124.9, 126.1, 129.3, 129.6, 133.1, 133.5, 134.2, 134.3, 139.4, 163.8 (d, J=240 Hz). MS (ESI+): m/z=412.1. ESI-HR-MS calcd for C₂₅H₁₆ClFN₃[M⁺]: 412.1011, found: 412.1008.

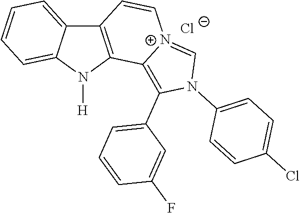 [SMILES: Fc1cccc(-c2c3c4[nH]c5ccccc5c4cc[n+]3cn2-c2ccc(Cl)cc2)c1 ; [Cl-]]

1-(3-Fluorophenyl)-2-(4-fluorophenyl)-2,11-dihydroimidazo[1′,5′:1,2]pyrido[3,4-b]indol-4-ium (8aid). Yield: 86% (0.383 g from 0.3 g); a white solid; mp: >250° C.; R_{f}=0.45 (MeOH:EtOAc, 5:5. v/v); IR (KBr) νₘₐₓ: 3346 cm⁻¹. ¹H NMR (400 MHz, DMSO-d⁶): δ (ppm)=7.31 (s, 2H), 7.42-7.45 (m, 5H), 7.58 (d, J=6.2 Hz, 1H), 7.66 (d, J=7.5 Hz, 3H), 8.18 (d, J=6.7 Hz, 2H), 8.59 (d, J=7.0 Hz, 1H), 10.66 (s, 1H). ¹³C NMR (100 MHz, DMSO-d⁶): δ (ppm)=113.6, 114.0, 116.6, 118.0, 118.2, 118.7, 118.9, 120.3, 120.7, 121.5, 122.2, 122.7, 124.9, 126.1, 127.2, 127.3, 128.0, 129.6, 130.0, 130.5, 132.0, 139.6, 162.0, 164.3 (d, J=60 Hz). MS (ESI+): m/z=396.1. ESI-HR-MS calcd for C₂₅H₁₆F₂N₃[M⁺]: 396.1307, found: 396.1301.

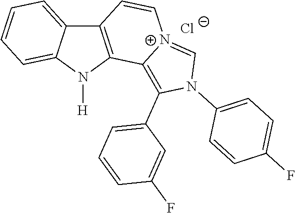 [SMILES: Fc1ccc(-n2c[n+]3ccc4c5ccccc5[nH]c4c3c2-c2cccc(F)c2)cc1 ; [Cl-]]

1-(2-Bromo-4-fluorophenyl)-2-(4-chlorophenyl)-2,11-dihydroimidazo[1′,5′:1,2]pyrido[3,4-b]indol-4-ium (8ajc). Yield: 78% (0.334 g from 0.3 g); a white solid; mp: 310-312° C.; R_{f}=0.43 (MeOH:EtOAc, 5:5. v/v); IR (KBr) νₘₐₓ: 3340 cm⁻¹. ¹H NMR (400 MHz, DMSO-d⁶): δ (ppm)=7.32 (t, J=7.5 Hz, 1H), 7.43 (t, J=7.6 Hz, 1H), 7.48-7.53 (m, 1H), 7.59 (d, J=8.7 Hz, 2H), 7.67 (t, J=8.8 Hz, 3H), 7.79-7.83 (m, 1H), 7.87-7.89 (m, 1H), 8.19-8.22 (m, 2H), 8.64 (d, J=7.2 Hz, 1H), 10.84 (s, 1H), 11.67 (s, 1H). ¹³C NMR (100 MHz, DMSO-d⁶): δ (ppm)=113.4, 114.0, 114.1, 116.6, 116.8, 120.5, 120.6, 121.3, 121.6, 121.7, 122.5, 122.7, 126.2, 128.8, 129.7, 130.2, 132.8, 136.0, 137.0, 139.6, 162.8, 168.1. MS (ESI+): m/z=490.0. ESI-HR-MS calcd for C₂₅H₁₅BrClFN₃[M⁺]: 490.0116, found: 490.0119.

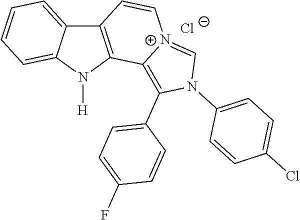 [SMILES: Fc1ccc(-c2c3c4[nH]c5ccccc5c4cc[n+]3cn2-c2ccc(Cl)cc2)cc1 ; [Cl-]]

2-(4-Fluorophenyl)-1-(p-tolyl)-2,11-dihydroimidazo[1′,5′:1,2]pyrido[3,4-b]indol-4-ium chloride (8akd). Yield: 73% (0.326 g from 0.3 g); light yellow solid; mp: 278-279° C.; R_{f}=0.47 (MeOH:EtOAc, 5:5. v/v); IR (KBr) νₘₐₓ: 93360 cm⁻¹. ¹H NMR (400 MHz, DMSO-d⁶): δ (ppm)=2.68 (s, 3H), 7.32 (t, J=6.4 Hz, 1H), 7.44 (t, J=8.1 Hz, 3H), 7.59-7.64 (m, 5H), 7.72 (s, 1H), 7.86 (s, 1H), 8.63 (s, 2H), 10.77 (s, 1H), 11.59 (s, 1H). ¹³C NMR (100 MHz, DMSO-d⁶): δ (ppm)=21.2, 113.5, 113.9, 114.1, 116.7, 117.1, 117.3, 120.3, 120.5, 121.6, 122.5, 122.6, 124.5, 124.8, 124.9, 126.1, 129.7, 129.9, 130.4, 133.0, 133.6, 139.4, 161.9, 164.5. MS (ESI+): m/z=392.1. ESI-HR-MS calcd for C₂₆H₁₉FN₃[M⁺]: 392.1558, found: 392.1560.

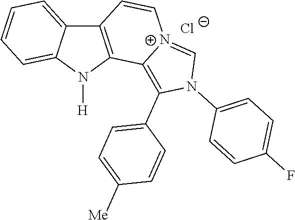 [SMILES: Cc1ccc(-c2c3c4[nH]c5ccccc5c4cc[n+]3cn2-c2ccc(F)cc2)cc1 ; [Cl-]]

2-(4-Fluorophenyl)-1-(4-nitrophenyl)-2,11-dihydrolmidazo[1′,5′:1,2]pyrido[3,4-b]indol-4-ium chloride (8ald). Yield: 74% (0.317 g from 0.3 g); yellow solid; mp: 278-280° C.; R_{f}=0.39 (MeOH:EtOAc, 5:5. v/v); IR (KBr) νₘₐₓ: 3389 cm⁻¹. ¹H NMR (400 MHz, DMSO-d⁶): δ (ppm)=7.33 (t, J=7.2 Hz, 1H), 7.48 (t, J=7.9 Hz, 3H), 7.68 (d, J=8.2 Hz, 3H), 7.80 (d, J=7.8 Hz, 2H), 8.21-8.24 (m, 2H), 8.43 (d, J=7.9 Hz, 2H), 8.65 (d, J=6.6 Hz, 1H), 10.76 (s, 1H), 11.52 (s, 1H) ¹³C NMR (100 MHz, DMSO-d⁶): δ (ppm)=113.5, 114.1, 114.6, 116.9, 117.2, 117.4, 120.4, 121.0, 121.5, 121.6, 122.6, 124.6, 125.1, 126.4, 130.0, 130.1, 130.3, 131.9, 133.1, 139.5, 148.8, 163.5 (d, J=10 Hz). MS (ESI+): m/z=423.1. ESI-HR-MS calcd for C₂₅H₁₆FN₄O₂[M⁺]: 423.1252, found: 423.1257.

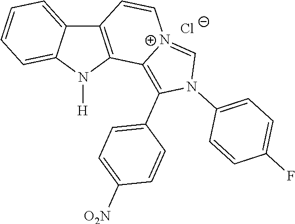 [SMILES: O=[N+]([O-])c1ccc(-c2c3c4[nH]c5ccccc5c4cc[n+]3cn2-c2ccc(F)cc2)cc1 ; [Cl-]]

1-(Benzofuran-2-yl)-2-phenyl-2,11-dihydroimidazo[1′,5′:1,2]pyrido[3,4-b]indol-4-ium chloride (8amd): Yield: 82% (0.343 g from 0.3 g); a white solid; mp: >350° C.; R_{f}=0.45 (MeOH:EtOAc, 5:5. v/v); IR (KBr) νₘₐₓ: 3366 cm. ¹H NMR (400 MHz, DMSO-d⁶): δ (ppm)=6.93 (s, 1H), 7.35-7.45 (m, 2H), 7.47-7.53 (m, 2H), 7.55-7.59 (m, 2H), 7.73-7.79 (m, 3H), 7.85-7.88 (m, 2H), 8.26 (t, J=7.1 Hz, 2H), 8.58 (t, J=7.2 Hz, 1H), 10.61 (s, 1H), 11.87 (s, 1H). ¹³C NMR (100 MHz, DMSO-d⁶): δ (ppm)=111.0, 112.6, 115.4, 117.1, 117.4, 117.6, 112.6, 124.2, 124.4, 126.5, 128.1, 129.8, 129.9, 131.7, 139.7, 155.5. MS (ESI+): m/z=400.1. ESI-HR-MS calcd for C₂₇H₁₈N₃O[M⁺]: 400.1444, found: 400.1449.

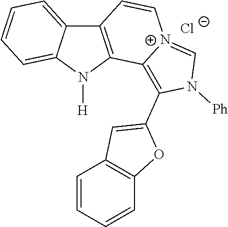 [SMILES: c1ccc(-n2c[n+]3ccc4c5ccccc5[nH]c4c3c2-c2cc3ccccc3o2)cc1 ; [Cl-]]

2-(4-Fluorophenyl)-1-(thiophen-2-yl)-2,11-dihydroimidazo[1′,5′:1,2]pyrido[3,4-b]indol-4-ium chloride (Sand). Yield: 85% (0.384 g from 0.3 g); a light yellow solid; mp: 325-327° C.; R_{f}=0.41 (MeOH:EtOAc, 5:5. v/v); IR (KBr) νₘₐₓ: 3377 cm⁻¹. ¹H NMR (400 MHz, DMSO-d⁶): δ (ppm)=7.29-7.34 (m, 2H), 7.43-7.50 (m, 4H), 7.73 (t, J=6.6 Hz, 3H), 7.95 (d, J=4.8 Hz, 1H), 8.19-8.21 (m, 2H), 8.61 (d, J=7.1 Hz, 1H), 10.71 (s, 1H), 11.53 (s, 1H) ¹³C NMR (100 MHz, DMSO-d⁶): δ (ppm)=104.2, 109.1, 113.8 (d, J=20 Hz), 114.4, 116.9, 117.2 (d, J=10 Hz), 120.3, 121.3, 121.6, 122.6, 124.0, 124.6, 126.2, 128.9, 130.1, 130.2, 130.3, 130.4, 132.5, 134.1, 136.9, 139.5, 143.8, 145.9. MS (ESI+): m/z=384.0. ESI-HR-MS calcd for C₂₃H₁₅FN₃S [M⁺]: 384.0965, found: 384.0969.

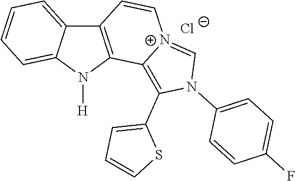 [SMILES: Fc1ccc(-n2c[n+]3ccc4c5ccccc5[nH]c4c3c2-c2cccs2)cc1 ; [Cl-]]

2-(4-Fluorophenyl)-1-(4-methoxyphenyl)-2,11-dihydroimidazo[1′,5′:1,2]pyrido[3,4-b]indol-4-ium (8aod). Yield: 90% (0.463 g from 0.3 g); a light yellow solid; mp: 255-257° C.; R_{f}=0.46 (MeOH:EtOAc, 5:5. v/v); IR (KBr) νₘₐₓ: 3388 cm⁻¹. ¹H NMR (400 MHz, DMSO-d⁶): δ (ppm)=3.55 (s, 3H), 3.87 (s, 3H), 6.69 (d, J=2.2 Hz, 1H), 6.72-6.74 (m, 1H), 7.28 (t, J=7.5 Hz, 1H), 7.37-7.40 (m, 2H), 7.43 (d, J=8.7 Hz, 2H), 7.53-7.57 (m, 2H), 7.68 (d, J=8.1 Hz, 1H), 8.12-8.17 (m, 2H), 8.54 (d, J=7.2 Hz, 1H), 10.63 (s, 1H), 11.35 (s, 1H). ¹³C NMR (100 MHz, DMSO-d⁶): δ (ppm)=56.0, 56.1, 99.6, 105.5, 107.0, 113.3, 113.6 (d, J=20 Hz), 116.8 (d, J=10 Hz), 117.0, 120.1, 120.4, 120.9, 121.4, 122.6, 125.1, 125.8, 128.9, 129.0, 129.2, 131.1, 134.3, 139.4, 159.6, 161.8, 163.8 (d, J=80 Hz). MS (ESI+): m/z=438.1. ESI-HR-MS calcd for C₂₇H₂₁FN₃O₂[M⁺]: 438.1612, found: 438.1616.

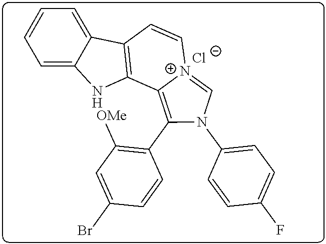 [SMILES: COc1cc(Br)ccc1-c1c2c3[nH]c4ccccc4c3cc[n+]2cn1-c1ccc(F)cc1 ; [Cl-]]

2-(4-Fluorophenyl)-1-(3,4,5-trimethoxyphenyl)-2,11-dihydroimidazo[1′,5′:1,2]pyrido[3,4-b]indol-4-ium chloride (8apd). Yield: 76% (0.317 g from 0.3 g); a brown solid; mp: 292-294° C.; R_{f}=0.40 (MeOH:EtOAc, 5:5. v/v); IR (KBr) μₘₐₓ: 3358 cm⁻¹. ¹H NMR (400 MHz, DMSO-d⁶): δ (ppm)=3.66 (s, 6H), 3.77 (d, J=0.93 Hz, 3H), 6.79 (s, 2H), 7.32 (t, J=7.1 Hz, 1H), 7.41 (d, J=7.2 Hz, 1H), 7.48 (t, J=6.9 Hz, 2H), 7.68-7.74 (m, 3H), 8.18 (t, J=6.6 Hz, 2H), 8.59 (d, J=7.1 Hz, 1H), 10.65 (s, 1H), 11.54 (s, 1H). ¹³C NMR (100 MHz, DMSO-d⁶): δ (ppm)=56.0, 56.1, 99.6, 105.5, 107.0, 113.3, 113.5, 113.7, 116.7, 116.8, 117.0, 120.1, 120.6 (d, J=50 Hz), 121.4, 122.6, 125.1, 125.8, 128.9, 129.0, 129.2, 131.1, 134.3, 139.4, 159.6, 161.8, 163.8 (d, J=80 Hz). MS (ESI+): m/z=468.1. ESI-HR-MS calcd for C₂₇H₂₃FN₃O₃[M⁺]: 468.1718, found: 468.1720.

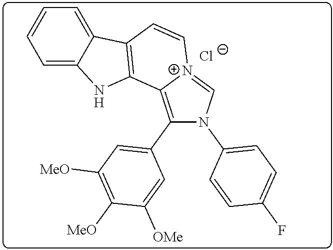 [SMILES: COc1cc(-c2c3c4[nH]c5ccccc5c4cc[n+]3cn2-c2ccc(F)cc2)cc(OC)c1OC ; [Cl-]]

1-(4-Bromophenyl)-2-(4-chlorophenyl)-5-(methoxycarbonyl)-2,11-dihydroimidazo[1′,5′:1,2]pyrido[3,4-b]indol-4-iumchloride (8bbc). Yield: 64% (0.265 g from 0.3 g); a pale yellow solid; mp: 121-123° C.; R_{f}=0.46 (MeOH:EtOAc, 5:5. v/v); IR (KBr) νₘₐₓ: 1719, 3456 cm⁻¹. ¹H NMR (400 MHz, DMSO-d⁶): δ (ppm)=4.02 (s, 3H), 7.31-7.35 (m, 1H), 7.41-7.45 (m, 3H), 7.52-7.56 (m, 2H), 7.59-7.65 (m, 3H), 7.72-7.75 (m, 2H), 8.33 (d, J=7.9 Hz, 1H), 9.12 (s, 1H), 10.71 (s, 1H), 11.75 (s, 1H). ¹³C NMR (100 MHz, DMSO-d⁶): δ (ppm)=53.7, 113.3, 113.9, 115.9, 120.6, 120.8, 122.4, 122.7, 123.0, 123.4, 124.4, 125.5, 126.9, 128.6, 129.7, 130.0, 130.4, 132.8, 133.2, 133.7, 136.0, 139.9, 162.0. MS (ESI+): m/z=530.0. ESI-HR-MS calculated for C₂₇H₁₈BrClN₃O₂ [M⁺]: 530.0265, found: 530.0270.

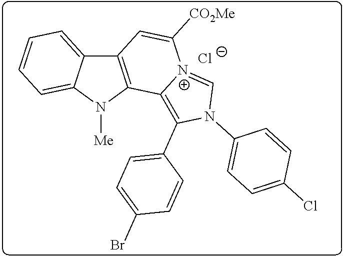 [SMILES: COC(=O)c1cc2c3ccccc3n(C)c2c2c(-c3ccc(Br)cc3)n(-c3ccc(Cl)cc3)c[n+]12 ; [Cl-]]

1-(4-Bromophenyl)-2-(4-fluorophenyl)-5-(methoxycarbonyl)-2,11-dihydroimidazo[1′,5′:1,2]pyrido[3,4-b]indol-4-iumchloride (8bbd): Yield: 71% (0.286 g from 0.3 g); a pale yellow solid; mp: 127-129° C.; R_{f}=0.43 (MeOH:EtOAc, 5:5. v/v); IR (KBr) νₘₐₓ: 1722, 3455 cm⁻¹. ¹H NMR (400 MHz, DMSO-d⁶): δ (ppm)=5.27 (s, 3H), 7.11-7.18 (m, 1H), 7.25 (s, 1H), 7.31 (s, 1H), 7.46 (s, 2H), 7.66 (s, 2H), 7.76-7.78 (m, 2H), 8.19 (s, 1H), 8.59 (d, J=6.4 Hz, 1H), 8.77 (s, 1H), 10.66 (s, 1H), 11.42 (s, 1H), 13.72 (s, B 1H). ¹³C NMR (100 MHz, DMSO-d⁶): δ (ppm)=49.2, 113.5, 114.0, 116.8, 117.0, 119.8, 120.3, 120.4, 121.5, 122.5, 124.5, 124.8, 125.3, 126.1, 127.0, 129.7, 130.0, 130.4, 133.0, 133.5, 137.5, 139.4, 149.8, 161.9, 163.3 (d, J=253.6 Hz). MS (ESI+): m/z=514.0. ESI-HR-MS calculated for C₂₇H₁₈BrFN₃O₂ [M⁺]: 514.0561, found: 514.0569.

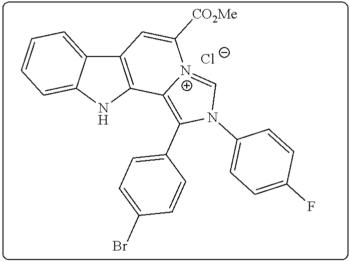 [SMILES: COC(=O)c1cc2c3ccccc3[nH]c2c2c(-c3ccc(Br)cc3)n(-c3ccc(F)cc3)c[n+]12 ; [Cl-]]

2-(4-Fluorophenyl)-5-(methoxycarbonyl)-1-(p-tolyl)-2,11-dihydroimidazo[1′,5′:1,2]pyrido[3,4-b]indol-4-iumchloride (8bkd). Yield: 67% (0.283 g from 0.3 g); a pale yellow solid; mp: 215-217° C.; R_{f}=0.47 (MeOH:EtOAc, 5:5. v/v); IR (KBr) νₘₐₓ: 1724, 3453 cm⁻¹. ¹H NMR (400 MHz, DMSO-d⁶): δ (ppm)=2.41 (s, 3H), 4.08 (s, 3H), 7.36-7.49 (m, 8H), 7.63-7.66 (m, 2H), 7.73 (d, J=8.2 Hz, 1H), 8.37-8.39 (m, 1H), 9.17 (s, 1H), 10.70 (s, 1H), 11.67 (s, 1H). ¹³C NMR (100 MHz, DMSO-d⁶): δ (ppm)=21.6, 53.7, 79.8, 113.1, 114.1, 115.8, 116.7, 116.9, 120.2, 120.7, 121.5, 122.4, 122.6, 123.0, 125.8, 126.7, 128.9, 130.3, 130.4, 130.6, 131.5, 139.9, 140.9, 162.1 (d, J=249.4 Hz), 162.6. MS (ESI+): m/z=450.1. ESI-HR-MS calculated for C₂₈H₂₁FN₃O₂ [M⁺]: 450.1612, found: 450.1618.

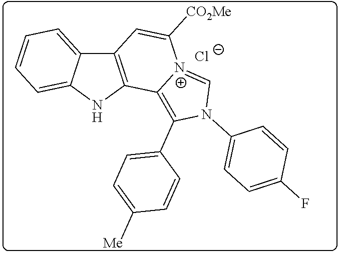 [SMILES: COC(=O)c1cc2c3ccccc3[nH]c2c2c(-c3ccc(C)cc3)n(-c3ccc(F)cc3)c[n+]12 ; [Cl-]]

8-Bromo-1-(2-bromophenyl)-2-phenyl-2,11-dihydrolmidazo[1′,5′:1,2]pyrido[3,4-b]indol-4-ium chloride (8ccd). Yield: 69% (0.266 g from 0.3 g); a light yellow solid; mp: 298-301° C.; R_{f}=0.44 (MeOH:EtOAc, 5:5. v/v); IR (KBr) νₘₐₓ: 3405 cm⁻¹. ¹H NMR (400 MHz, DMSO-d⁶): δ (ppm)=7.31 (t, J=7.4 Hz, 1H), 7.43 (t, J=8.5 Hz, 3H), 7.52 (d, J=8.2 Hz, 2H), 7.58 (t, J=7.6 Hz, 2H), 7.65 (d, J=7.9 Hz, 1H), 7.80 (d, J=8.1 Hz, 2H), 8.18 (d, J=5.0 Hz, 2H), 8.59 (d, J=6.9 Hz, 1H), 10.67 (s, 1H), 11.36 (s, 1H). ¹³C NMR (100 MHz, DMSO-d⁶): δ (ppm)=113.6, 114.0, 114.1, 116.6, 120.4, 121.5, 121.6, 122.6, 124.8, 126.1, 126.2, 126.3, 128.8, 129.0, 129.6, 130.1, 132.9, 133.8, 135.2, 135.9, 139.9. MS (ESI+): m/z=515.9. ESI-HR-MS calcd for C₂₅H₁₆Br₂N₃[M⁺]: 515.9705, found: 515.9711.

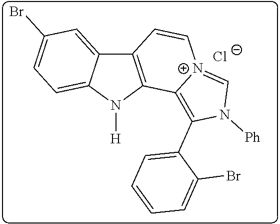 [SMILES: Brc1ccc2[nH]c3c(cc[n+]4cn(-c5ccccc5)c(-c5ccccc5Br)c34)c2c1 ; [Cl-]]

2-Benzyl-1-(4-bromophenyl)-2,11-dihydroimidazo[1′,5′:1,2]pyrido[3,4-b]indol-4-ium chloride (9aaa). Yield: 82% (0.343 g from 0.3 g); a light yellow solid; mp: 283-285° C.; R_{f}=0.49 (MeOH:EtOAc, 5:5. v/v); IR (KBr) νₘₐₓ: 3476 cm⁻¹. ¹H NMR (400 MHz, DMSO-d⁶): δ (ppm)=5.83 (s, 2H), 7.28 (t, J=7.5 Hz, 1H), 7.39 (t, J=7.8 Hz, 3H), 7.56 (d, J=8.4 Hz, 3H), 7.61 (d, J=8.2 Hz, 1H), 7.66 (d, J=8.1 Hz, 2H), 7.81 (d, J=8.3 Hz, 2H), 8.08 (d, J=7.2 Hz, 1H), 8.15 (d, J=7.9 Hz, 1H), 8.55 (d, J=7.2 Hz, 1H), 10.41 (s, 1H), 11.29 (s, 1H). ¹³C NMR (100 MHz, DMSO-d⁶): δ (ppm)=51.0, 113.4, 113.9, 117.1, 121.2, 121.4, 121.5, 122.6, 123.3, 124.4, 124.9, 125.3, 125.7, 125.8, 125.9, 129.0, 129.1, 129.2, 129.3, 130.2, 133.2, 133.6, 139.1, 139.3, 139.8. MS (ESI+): m/z=452.0. ESI-HR-MS calcd for C₂₆H₁₈BrN₃[M⁺]: 452.0757, found: 452.0759.

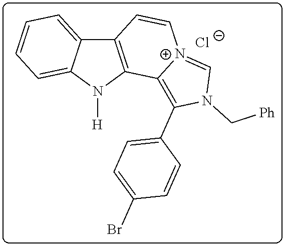 [SMILES: Brc1ccc(-c2c3c4[nH]c5ccccc5c4cc[n+]3cn2Cc2ccccc2)cc1 ; [Cl-]]

1-(4-Bromophenyl)-2-(4-fluorobenzyl)-2,11-dihydroimidazo[1′,5′:1,2]pyrido[3,4-b]indol-4-ium chloride (9aab). Yield: 75% (0.325 g from 0.3 g); a light yellow solid; mp: 172-174° C.; R_{f}=0.49 (MeOH:EtOAc, 5:5. v/v); IR (KBr) νₘₐₓ: 3390 cm⁻¹. ¹H NMR (400 MHz, DMSO-d⁶): δ (ppm)=5.62 (s, 2H), 7.16 (t, J=8.8 Hz, 2H), 7.20-7.24 (m, 2H), 7.28 (t, J=7.6 Hz, 1H), 7.39 (t, J=7.4 Hz, 1H), 7.55-7.61 (m, 1H), 7.85 (d, J=8.4 Hz, 2H), 8.06 (d, J=7.2 Hz, 1H), 8.13 (d, J=7.8 Hz, 1H), 8.47 (d, J=7.0 Hz, 1H), 10.11 (s, 1H), 11.24 (s, 1H). ¹³C NMR (100 MHz, DMSO-d⁶): δ (ppm)=50.9, 113.2, 113.6 (d, J=40 Hz), 116.0, 116.2, 117.1, 120.2, 121.1, 121.4, 121.5, 122.6, 124.5, 124.9, 125.2, 126.0, 128.8, 130.4, 130.5, 130.9, 131.0, 133.2, 133.6, 139.3, 162.5 (d, J=240 Hz). MS (ESI+): m/z=470.0. ESI-HR-MS calcd for C₂₆H₁₈BrFN₃[M⁺]: 470.0663, found: 470.0667.

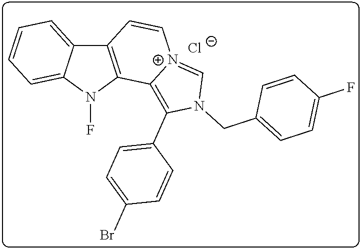 [SMILES: Fc1ccc(Cn2c[n+]3ccc4c5ccccc5n(F)c4c3c2-c2ccc(Br)cc2)cc1 ; [Cl-]]

1-(4-Bromophenyl)-2-(4-(tert-butyl)benzyl)-2,11-dihydroimidazo[1′,5′:1,2]pyrido[3,4-b]indol-4-ium chloride (9aac). Yield: 61% (0.284 g from 0.3 g); a white solid; mp: 251-253° C.; R_{f}=0.50 ((MeOH:EtOAc, 5:5. v/v); IR (KBr) νₘₐₓ 3388 cm⁻¹. ¹H NMR (400 MHz, DMSO-d⁶): δ (ppm)=1.25 (s, 9H), 5.57 (s, 2H), 7.10 (d, J=7.9 Hz, 2H), 7.33 (d, J=8.0 Hz, 2H), 7.42 (s, 2H), 7.58-7.65 (m, 2H), 7.85 (d, J=7.8 Hz, 3H), 8.06 (d, J=7.5 Hz, 1H), 8.14 (d, J=7.9 Hz, 1H), 8.48 (d, J=7.9 Hz, 1H), 10.14 (s, 1H), 11.26 (s, 1H). ¹³C NMR (100 MHz, DMSO-d⁶): δ (ppm)=51.3, 113.3, 113.4, 113.8, 117.0, 120.2, 120.9, 121.4, 121.5, 122.6, 124.5, 124.9, 125.2, 125.7, 126.0, 128.5, 128.7, 129.3, 131.4, 131.6, 133.3, 133.5, 133.7, 139.3, 151.6. MS (ESI+): m/z=508.1. ESI-HR-MS calcd for C₃₀H₂₇BrN₃[M⁺]: 508.1383, found: 508.1387.

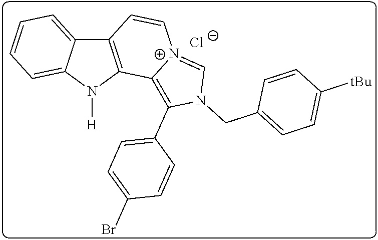 [SMILES: CC(C)(C)c1ccc(Cn2c[n+]3ccc4c5ccccc5[nH]c4c3c2-c2ccc(Br)cc2)cc1 ; [Cl-]]

1-(4-Bromophenyl)-2-(4-(trifluoromethyl)benzyl)-2,11-dihydroimidazo[1′,5′:1,2]pyrido[3,4-b]indol-4-ium chloride (9aad). Yield: 80% (0.380 g from 0.3 g); a white solid; mp: 267-269° C.; R_{f}=0.53 (MeOH:EtOAc, 5:5. v/v); IR (KBr) μₘₐₓ: 3404 cm⁻¹. ¹H NMR (400 MHz, DMSO-d⁶): δ (ppm)=5.81 (s, 2H), 7.30 (d, J=7.5 Hz, 1H), 7.38-7.42 (m, 3H), 7.56 (d, J=8.2 Hz, 2H), 7.61 (d, J=8.2 Hz, 1H), 7.66 (d, J=8.1 Hz, 2H), 7.81 (d, J=8.1 Hz, 2H), 8.09 (d, J=7.1 Hz, 1H), 8.16 (d, J=7.8 Hz, 1H), 8.55 (d, J=7.1 Hz, 1H), 10.32 (s, 1H). ¹³C NMR (100 MHz, DMSO-d⁶): δ (ppm)=51.0, 113.4, 113.9, 117.1, 121.2, 121.4, 121.5, 122.6, 123.3, 124.4, 124.9, 125.7, 125.8, 129.1, 129.2, 129.3, 130.3, 133.2, 133.6, 139.1, 139.3, 139.8. MS (ESI+): m/z=520.0. ESI-HR-MS calcd for C₂₇H₁₈BrF₃N₃+[M⁺]: 520.0631, found: 520.0638.

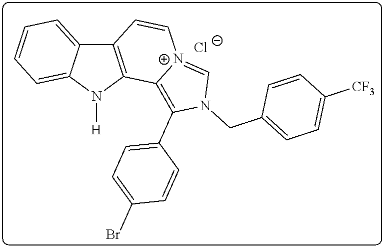 [SMILES: FC(F)(F)c1ccc(Cn2c[n+]3ccc4c5ccccc5[nH]c4c3c2-c2ccc(Br)cc2)cc1 ; [Cl-]]

2-(4-Methoxybenzyl)-1-phenyl-2,11-dihydroimidazo[1′,5′:1,2]pyrido[3,4-b]indol-4-ium chloride (9aae). Yield: 89% (0.394 g from 0.3 g); a light yellow solid; mp: 230-232° C.; R_{f}=0.53 (MeOH:EtOAc, 5:5. v/v); IR (KBr) μₘₐₓ: 3412 cm⁻¹. ¹H NMR (400 MHz, DMSO-d⁶): δ (ppm)=3.73 (d, J=3.4 Hz, 3H), 5.55 (s, 2H), 6.89 (d, J=8.7 Hz, 2H), 7.14 (d, J=8.7 Hz, 2H), 7.27 (t, J=7.8 Hz, 1H), 7.37-7.41 (m, 1H), 7.61 (t, J=8.5 Hz, 3H), 7.88 (d, J=8.4 Hz, 2H), 8.06 (d, J=7.2 Hz, 1H), 8.13 (d, J=7.9 Hz, 1H), 8.49 (d, J=7.2 Hz, 1H), 10.19 (m, 1H). ¹³C NMR (100 MHz, DMSO-d⁶): δ (ppm)=51.2, 55.7, 113.1, 113.4, 113.8, 114.3, 114.6, 117.0, 120.2, 120.9, 121.4, 121.5, 122.6, 124.6, 124.9, 125.2, 125.9, 128.6, 130.5, 133.2, 133.6, 139.3, 159.9. MS (ESI+): m/z=482.0. ESI-HR-MS calcd for C₂₇H₂₁BrClN₃O [M⁺]: 482.086, found: 482.0867.

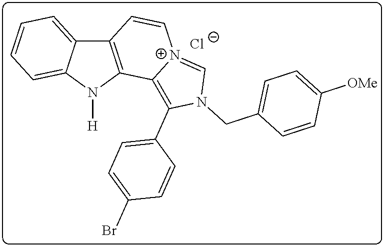 [SMILES: COc1ccc(Cn2c[n+]3ccc4c5ccccc5[nH]c4c3c2-c2ccc(Br)cc2)cc1 ; [Cl-]]

1-(4-Bromophenyl)-2-(2,5-difluorophenyl)-2,11-dihydroimidazo[1′,5′:1,2]pyrido[3,4-b]indol-4-ium chloride (9aaf). Yield: 69% (0.309 g from 0.3 g); a pale yellow solid; mp: 270-272° C.; R_{f}=0.43 (MeOH:EtOAc, 5:5. v/v); IR (KBr) μₘₐₓ: 3428 cm⁻¹. ¹H NMR (400 MHz, DMSO-d⁵): δ (ppm)=5.76 (s, 2H), 7.11 (t, J=7.2 Hz, 1H), 7.23 (d, J=5.8 Hz, 1H), 7.28 (d, J=7.6 Hz, 1H), 7.33-7.36 (m, 1H), 7.39 (t, J=7.5 Hz, 1H), 7.56-7.62 (m, 2H), 7.72-7.75 (m, 1H), 7.83 (d, J=8.3 Hz, 1H), 8.09 (d, J=7.2 Hz, 1H), 8.15 (d, J=7.8 Hz, 1H), 8.56 (d, J=7.1 Hz, 1H), 9.93 (s, 1H), 10.38 (s, 1H), 11.29 (s, 1H). ¹³C NMR (100 MHz, DMSO-d⁶): δ (ppm)=113.4, 113.8, 117.1, 117.4, 117.7, 118.0, 118.8, 119.1, 120.2, 121.1, 121.4, 122.5, 124.3, 124.8, 125.3, 125.9, 129.3, 133.2, 133.5, 139.3, 155.2, 155.9, 157.3, 157.6, 159.7. MS (ESI+): m/z=488.0. ESI-HR-MS calculated for C₂₆H₁₇BrF₂N₃[M⁺]: 488.0568, found: 488.0570.

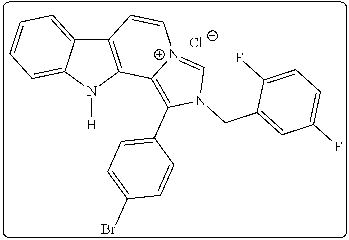 [SMILES: Fc1ccc(F)c(Cn2c[n+]3ccc4c5ccccc5[nH]c4c3c2-c2ccc(Br)cc2)c1 ; [Cl-]]

1-(4-Bromophenyl)-2-(3,4-dimethoxybenzyl)-2,11-dihydroimidazo[1′,5′:1,2]pyrido[3,4-b]indol-4-ium chloride (9aag). Yield: 78% (0.366 g from 0.3 g); a white solid; mp: 240-242° C.; R_{f}=0.41 (MeOH:EtOAc, 5:5. v/v); IR (KBr) νₘₐₓ: 3378 cm⁻¹. ¹H NMR (400 MHz, DMSO-d⁶): δ (ppm)=3.58 (s, 3H), 3.64 (s, 3H), 5.43 (s, 2H), 6.66-6.67 (m, 2H), 6.80-6.82 (m, 1H), 7.18 (t, J=7.6 Hz, 1H), 7.30 (t, J=7.7 Hz, 1H), 7.51 (s, 1H), 7.54 (t, J=8.3 Hz, 2H), 7.95 (d, J=7.2 Hz, 2H), 8.04 (d, J=7.8 Hz, 1H), 8.38 (d, J=7.3 Hz, 1H), 10.05 (s, 1H). ¹³C NMR (100 MHz, DMSO-d⁶): δ (ppm)=55.9, 56.0, 112.0, 112.3, 112.9, 113.1, 113.4, 113.7, 117.0, 120.1, 120.9, 121.3, 121.8, 122.6, 124.7, 124.8, 125.2, 125.9, 126.0, 128.6, 133.3, 133.7, 139.2, 149.1, 149.5. MS (ESI+): m/z=512.0. ESI-HR-MS calcd for C₂₈H₂₃BrN₃O₂+ [M⁺]: 512.0968, found: 512.0970.

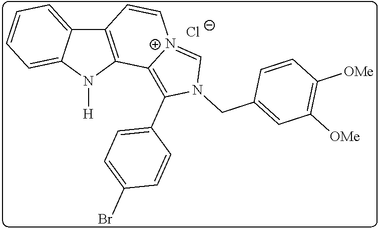 [SMILES: COc1ccc(Cn2c[n+]3ccc4c5ccccc5[nH]c4c3c2-c2ccc(Br)cc2)cc1OC ; [Cl-]]

1-(4-Chlorophenyl)-2-(4-methoxybenzyl)-2,11-dihydrolmidazo[1′,5′:1,2]pyrido[3,4-b]indol-4-ium chloride (9abe). Yield 79% (0.366 g from 0.3 g); a white solid; mp: 280-282° C.; R_{f}=0.51 (MeOH:EtOAc, 5:5. v/v); IR (KBr) νₘₐₓ: 3380 cm⁻¹. ¹H NMR (400 MHz, DMSO-d⁶): δ (ppm)=3.89 (s, 3H), 5.54 (s, 2H), 6.87 (d, J=8.4 Hz, 1H), 6.93 (d, J=8.3 Hz, 1H), 7.12 (d, J=8.5 Hz, 1H), 7.26 (t, J=7.3 Hz, 1H), 7.38 (t, J=7.6 Hz, 1H), 7.43 (d, J=8.3 Hz, 2H), 7.59 (d, J=8.0 Hz, 1H), 7.66-7.74 (m, 3H), 8.04 (d, J=7.2 Hz, 1H), 8.12 (d, J=7.9 Hz, 1H), 8.48 (d, J=7.1 Hz, 1H), 10.19 (s, 1H). ¹³C NMR (100 MHz, DMSO-d⁶): δ (ppm)=42.1, 55.6, 113.1, 113.4, 113.8, 114.3, 114.6, 117.0, 120.2, 121.0, 121.4, 122.6, 124.2, 124.8, 125.9, 126.6, 128.5, 130.3, 130.4, 131.0, 133.4, 136.2, 139.3, 159.7, 159.9. MS (ESI+): m/z=438.1. ESI-HR-MS calcd for C₂₇H₂₁ClN₃O [M⁺]: 438.1368, found: 438.1371.

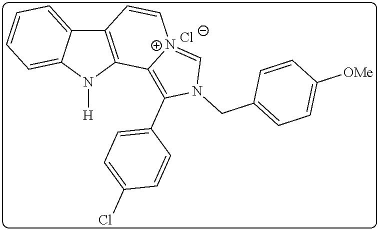 [SMILES: COc1ccc(Cn2c[n+]3ccc4c5ccccc5[nH]c4c3c2-c2ccc(Cl)cc2)cc1 ; [Cl-]]

1-(4-Chlorophenyl)-2-(4-(trifluoromethoxy)benzyl)-2,11-dihydroimidazo[1′,5′:1,2]pyrido[3,4-b]indol-4-ium chloride (9abh). Yield: 65% (0.336 g 41 rom 0.3 g); a light yellow solid; mp: 230-232° C.; R_{f}=0.44 (MeOH:EtOAc, 5:5. v/v); IR (KBr) νₘₐₓ: 3381 cm⁻¹. ¹H NMR (400 MHz, DMSO-d⁶): δ (ppm)=5.75 (s, 2H), 7.28 (s, 3H), 7.38 (d, J=6.9 Hz, 5H), 7.38 (d, J=6.9 Hz, 4H), 7.62-7.71 (m, 1H), 8.07 (d, J=6.8 Hz, 1H), 8.14 (d, J=7.5 Hz, 1H), 8.55 (d, J=6.6 Hz, 1H), 10.39 (s, 1H). ¹³C NMR (100 MHz, DMSO-d⁶): δ (ppm)=50.8, 113.2, 113.4, 113.8, 116.7, 117.1, 119.1, 119.2, 120.2, 121.2, 121.3, 121.4, 121.6, 121.7, 121.8, 122.6, 124.1, 124.3, 124.9, 125.9, 129.0, 130.2, 130.7, 139.7, 133.4, 133.7, 134.1, 136.2, 139.3, 148.7. MS (ESI+): m/z=492.1. ESI-HR-MS calcd for C₂₇H₁₈ClF₃N₃O [M⁺]: 492.1085, found: 492.1081.

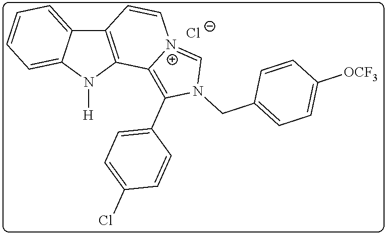 [SMILES: FC(F)(F)Oc1ccc(Cn2c[n+]3ccc4c5ccccc5[nH]c4c3c2-c2ccc(Cl)cc2)cc1 ; [Cl-]]

1-(4-Chlorophenyl)-2-(3,4-dimethoxybenzyl)-2,11-dihydroimidazo[1′,5′:1,2]pyrido[3,4-b]indol-4-ium chloride (9abg). Yield: 75% (0.278 g from 0.3 g); a white solid; mp: 240-242° C.; R_{f}=0.49 (MeOH:EtOAc, 5:5. v/v); IR (KBr) νₘₐₓ: 3370 cm⁻¹. ¹H NMR (400 MHz, DMSO-d⁶): δ (ppm)=3.83 (s, 3H), 3.85 (s, 3H), 5.62 (s, 2H), 6.83-6.85 (m, 1H), 6.86 (d, J=1.7 Hz, 1H), 6.97-7.01 (s, 1H), 7.03 (s, 1H), 7.06-7.08 (m, 1H), 7.33-7.34 (m, 1H), 7.46-7.49 (m, 1H), 7.69 (d, J=8.3 Hz, 1H), 7.80 (d, J=8.5 Hz, 1H), 7.84-7.86 (m, 1H), 8.13 (d, J=7.2 Hz, 1H), 8.22 (d, J=7.9 Hz, 1H), 8.57 (d, J=7.2 Hz, 1H), 10.29 (s, 1H). ¹³C NMR (100 MHz, DMSO-d⁶): δ (ppm)=51.6, 55.9, 56.0, 112.0, 112.2, 112.9, 113.1, 113.4, 113.7, 117.0, 120.2, 120.9, 121.3, 121.4, 121.7, 121.8, 122.6, 124.3, 124.9, 125.9, 126.0, 127.6, 128.6, 130.4, 133.5, 136.2, 139.3, 149.0, 149.1, 149.5. MS (ESI+): m/z=468.1. ESI-HR-MS calcd for C₂₈H₂₃ClN₃O₂ [M⁺]: 468.1473, found: 468.1470.

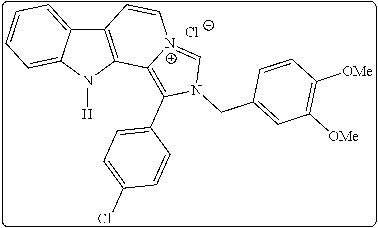 [SMILES: COc1ccc(Cn2c[n+]3ccc4c5ccccc5[nH]c4c3c2-c2ccc(Cl)cc2)cc1OC ; [Cl-]]

1-(4-Chlorophenyl)-2-(thiophen-3-ylmethyl)-2,11-dihydroimidazo[1′,5′:1,2]pyrido[3,4-b]indol-4-ium chloride (9acj). Yield: 80% (0.352 g from 0.3 g); a yellow solid; mp 310-312° C.; R_{f}=0.49 (MeOH:EtOAc, 5:5. v/v); IR (KBr) νₘₐₓ: 3369 cm⁻¹. ¹H NMR (400 MHz, DMSO-d⁶): δ (ppm)=5.90 (s, 2H), 6.98 (t, J=7.5 Hz, 1H), 7.05 (d, J=2.3 Hz, 1H), 7.25 (t, J=7.5 Hz, 1H), 7.37 (t, J=7.1 Hz, 1H), 7.54-7.61 (m, 2H), 7.73-7.78 (m, 3H), 8.04 (d, J=7.0 Hz, 1H), 8.11 (d, J=8.0 Hz, 1H), 8.53 (d, J=7.1 Hz, 1H), 10.39 (s, 1H), 11.26 (s, 1H). ¹³C NMR (100 MHz, DMSO-d⁶): δ (ppm)=46.2, 113.3, 113.4, 113.8, 117.0, 120.1, 120.9, 121.1, 121.3, 122.5, 123.9, 124.7, 125.9, 127.7, 128.5, 128.6, 129.8, 130.5, 133.5, 135.5, 136.3, 139.2. MS (ESI+): m/z=414.1. ESI-HR-MS calcd for C₂₄H₁₇ClN₃S [M⁺]: 414.0826, found: 414.0829.

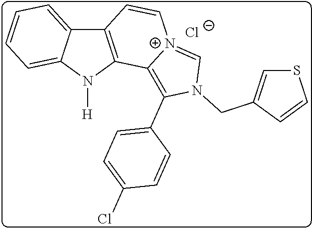 [SMILES: Clc1ccc(-c2c3c4[nH]c5ccccc5c4cc[n+]3cn2Cc2ccsc2)cc1 ; [Cl-]]

### Example 2

### Synthesis of 2-(4-Fluorophenyl)-5-(methoxycarbonyl)-1-methyl-2,11-dihydroimidazo[1′,5′:1,2]pyrido[3,4-b]indol-4-ium chloride (8bpd)

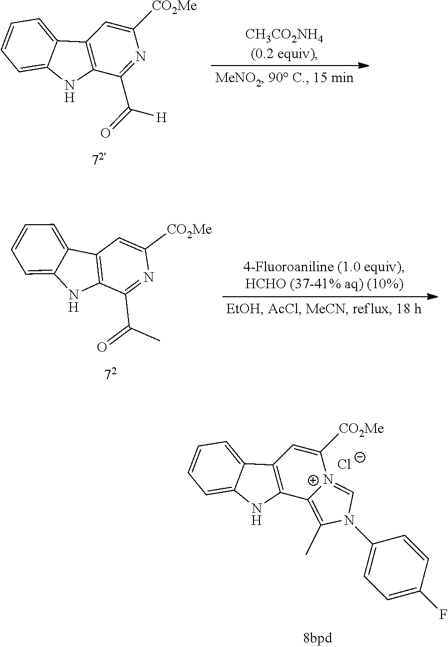 [SMILES: COC(=O)c1cc2c([nH]c3ccccc32)c(C=O)n1 ; "CH3CO2NH4(0.2 equiv)," ; COC(=O)c1cc2c([nH]c3ccccc32)c(C(C)=O)n1 ; COC(=O)c1cc2c3ccccc3[nH]c2c2c(C)n(-c3ccc(F)cc3)c[n+]12 ; [Cl-]]

Step A-1. To a stirred mixture of 1-formyl-9H-β-carboline (0.3 g, 1.2 mmol) in nitromethane (4.0 mL) was added NH₄OAc (0.018 g, 0.24 mmol) and the reaction was heated at 90° C. for 15 min. Upon completion the reaction was quenched with water. The aqueous layer was further extracted with EtOAc (2×25 mL). The organic layers were combined and washed with brine, dried over anhyd. Na₂SO₄ and concentrated under reduced pressure to obtain a residue which was purified by column chromatography on silica gel using hexanes/EtOAc (6:4, v/v) to obtain pure d (0.27 g from 0.3 g, 85%) as a brown solid.

Step B. To a stirred solution of formaldehyde 37-41% solution in water (50 μL, 1.6 mmol) and 4-fluoroaniline d (0.127 mL, 1.34 mmol) was added a freshly prepared ethanolic hydrochloride (2.0 mL) and the reaction was allowed to proceed for 20 min. A solution of 1-acetyl-9H-β-carboline (0.3 g, 1.11 mmol) in acetonitrile (9 mL) was added and the reaction was continued for 18 h at room temperature. After completion of the reaction, as monitored by TLC, the solvent was evaporated under reduced pressure to afford the crude product, which was purified by column chromatography (MeOH/EtOAc, 3:7, R_{f}=0.45) to afford 82% (0.375 g from 0.3 g) as a white solid. The product afforded by column chromatography was further crystallized by triturating with diethyl ether/ethanol mixture to give pure solid product. 2-(4-Fluorophenyl)-5-(methoxycarbonyl)-1-methyl-2,11-dihydroimidazo[1′,5′:1,2]pyrido[3,4-b]indol-4-ium chloride (8bpd). Yield: 82% (0.375 g from 0.3 g); a white solid; mp: 265° C.; R_{f}=0.45 (MeOH:EtOAc, 5:5. v/v); IR (KBr) vax: 1720, 3346 cm⁻¹. ¹H NMR (400 MHz, DMSO-d⁶): δ (ppm)=3.36 (s, 3H), 4.05 (s, 3H), 7.40 (t, J=7.8 Hz, 1H), 7.52 (t, J=8. 0 Hz, 1H), 7.63 (t, J=8.7 Hz, 2H), 7.85-7.91 (m, 3H), 8.35 (d, J=7.9 Hz, 1H), 9.07 (s, 1H), 10.52 (s, 1H), 13.51 (s, 1H). ¹³C NMR (100 MHz, DMSO-d⁶): δ (ppm)=10.7, 53.6, 93.1, 105.4, 111.9, 113.4, 115.7, 117.3, 117.5, 120.4, 120.8, 122.5, 122.6, 122.9, 123.2, 129.5, 130.1, 139.9, 164.6 (d, J=510 Hz), MS (ESI+): m/z=375.1. ESI-HR-MS calcd for C₂₂H₁₇FN₃O₂ [M⁺+H]: 375.1378, found: 375.1375.

### Example 3

### 1-(4-Bromophenyl)-2-(4-chlorophenyl)-11-methyl-2,11 dihydroimidazo[1′,5′:1,2]pyrido[3,4-b]indol-4-iumchloride (8dbc)

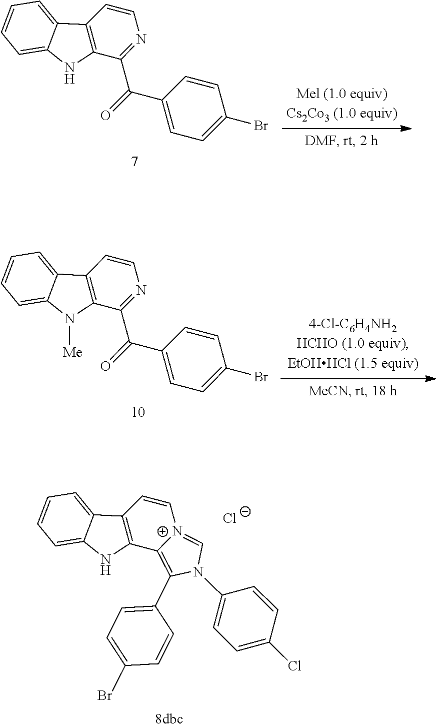 [SMILES: O=C(c1ccc(Br)cc1)c1nccc2c1[nH]c1ccccc12 ; Cn1c2ccccc2c2ccnc(C(=O)c3ccc(Br)cc3)c21 ; Clc1ccc(-n2c[n+]3ccc4c5ccccc5[nH]c4c3c2-c2ccc(Br)cc2)cc1 ; [Cl-]]

Step A-3. To a stirred solution of (4-bromophenyl)(9H-pyrido[3,4-b]indol-1-yl)methanone 1 (0.5 g, 1.4 mmol) in DMF (5 mL), cesium carbonate (0.46 g, 1.4 mmol) was added and the reaction was continued at room temperature for 10 minute. Thereafter, methyl iodide (0.088 mL, 1.4 mmol) was added and the stirring was continued for another 2 h. Upon completion, the reaction mixture was poured on to the crushed ice to obtain a solid precipitate (crude product) that was filtered and dried under vacuum. The product (9-methyl-9H-pyrido[3,4-b]indol-1-yl)(phenyl)methanone 10 was used for further reaction without any purification.

Step B. To a stirred solution of formaldehyde 37-41% solution in water (108 μL, 1.23 mmol) and 4-chloroaniline (0.12 g, 0.98 mmol) was added a freshly prepared ethanolic hydrochloride (2.0 mL) and the reaction was allowed to proceed for 20 min. A solution of (4-bromophenyl)(9H-pyrido[3,4-b]indol-1-yl)methanone (0.30 g, 0.82 mmol) in acetonitrile (9 mL) was added and the reaction was heated at 45° C. until it was dissolved and then continued for 18 h at room temperature. After completion of the reaction, as monitored by TLC, the solvent was evaporated under reduced pressure to afford the crude product, which was purified by column chromatography (MeOH/EtOAc, 3:7) to afford 0.325 g (88%) as a white solid. The product was recrystallized by triturating with diethyl ether/ethanol mixture to give pure solid product. 1-(4-Bromophenyl)-2-(4-chlorophenyl)-11-methyl-2,11 dihydrolmidazo[1′,5′:1,2]pyrido[3,4-b]indol-4-iumchloride (8dbc). Yield: 88% (0.325 g from 0.30 g); a white solid; mp: 217-219° C.; R_{f}=0.46 (MeOH:EtOAc, 5:5. v/v); ¹H NMR (400 MHz, DMSO-d⁶): δ (ppm)=3.28 (s, 3H), 7.35-7.39 (m, 1H), 7.40-7.45 (m, 2H), 7.51 (t, J=7.3 Hz, 1H), 7.62 (d, J=8.3 Hz, 2H), 7.66 (d, J=8.4 Hz, 1H), 7.68-7.22 (m, 2H), 7.75 (d, J=8.4 Hz, 2H), 8.19-8.25 (m, 2H), 8.62 (d, J=7.2 Hz, 1H), 10.70 (s, 1H). ¹³C NMR (100 MHz, DMSO-d⁶): δ (ppm)=34.3, 111.4, 113.5, 114.5, 116.9, 120.4, 121.5, 121.8, 122.4, 122.8, 125.3, 126.3, 126.8, 126.9, 129.7, 129.8, 130.0, 132.5, 133.1, 135.4, 136.0, 140.4. MS (ESI+): m/z=486.0. ESI-HR-MS calculated for C₂₆H₁₈BrClN₃ [M⁺]: 486.0367, found: 486.0373.

The following derivarives were prepared via identical experimental procedure.

1-(4-Bromophenyl)-2-(4-fluorophenyl)-11-methyl-2,11-dihydroimidazo[1′,5′:1,2]pyrido[3,4-b]indol-4-iumchloride (8dbd). Yield: 84% (0.301 g from 0.3 g); a white solid; mp: 223-225° C.; R_{f}=0.45 (MeOH:EtOAc, 5:5. v/v); ¹H NMR (400 MHz, DMSO-d⁶): δ (ppm)=4.07 (s, 3H), 7.33-7.36 (m, 1H), 7.43-7.56 (m, 4H), 7.63-7.68 (m, 4H), 7.76 (d, J=7.7 Hz, 2H), 8.34 (d, J=7.7 Hz, 1H), 9.13 (s, 1H), 10.71 (s, 1H), 11.84 (s, 1H). ¹³C NMR (100 MHz, DMSO-d⁶): δ (ppm)=53.5, 113.4, 113.5, 114.6, 116.8, 116.9, 117.1, 120.6, 122.2, 122.4, 123.4, 124.1, 124.3, 125.1, 126.5, 130.1, 130.3, 130.9, 132.9, 133.8, 134.3, 162.2, 163.2 (d, J=249.1 Hz). MS (ESI+): m/z=470.0. ESI-HR-MS calculated for C₂₆H₁₈BrFN₃ [M⁺]: 470.0663, found: 470.0668.

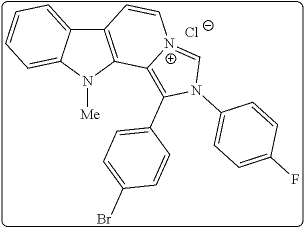 [SMILES: Cn1c2ccccc2c2cc[n+]3cn(-c4ccc(F)cc4)c(-c4ccc(Br)cc4)c3c21 ; [Cl-]]

1,2-Bis(4-chlorophenyl)-11-methyl-2,11-dihydroimidazo[1′,5′:1,2]pyrido[3,4-b]indol-4-iumchloride (8ddc). Yield: 81% (0.306 g from 0.3 g); a white solid; mp: 334-337° C.; R_{f}=0.44 (MeOH:EtOAc, 5:5. v/v); ¹H NMR (400 MHz, DMSO-d⁶): δ (ppm)=3.19 (s, 3H), 7.26-7.30 (m, 1H), 7.39-7.44 (m, 1H), 7.52-7.54 (m, 2H), 7.56-7.59 (m, 5H), 7.61-7.63 (m, 2H), 8.11-8.16 (m, 2H), 8.55 (d, J=7.1 Hz, 1H), 10.67 (s, 1H). ¹³C NMR (100 MHz, DMSO-d⁶): δ (ppm)=34.3, 111.4, 113.5, 114.4, 116.9, 120.4, 121.5, 121.8, 122.3, 122.8, 126.3, 126.4, 126.8, 129.6, 129.7, 129.8, 130.0, 133.1, 135.2, 136.0, 136.5, 140.5. MS (ESI+): m/z=442.0. ESI-HR-MS calculated for C₂₆H₁₈Cl₂N₃ [M⁺]: 442.0872, found: 442.0878.

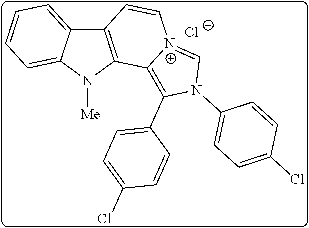 [SMILES: Cn1c2ccccc2c2cc[n+]3cn(-c4ccc(Cl)cc4)c(-c4ccc(Cl)cc4)c3c21 ; [Cl-]]

### Example 4

### Synthesis of 1-phenyl-2-(4-fluorophenyl)-2,13-dihydroimidazo[1,5-a]indolo[2,3-c]quinolin-4-ium chloride

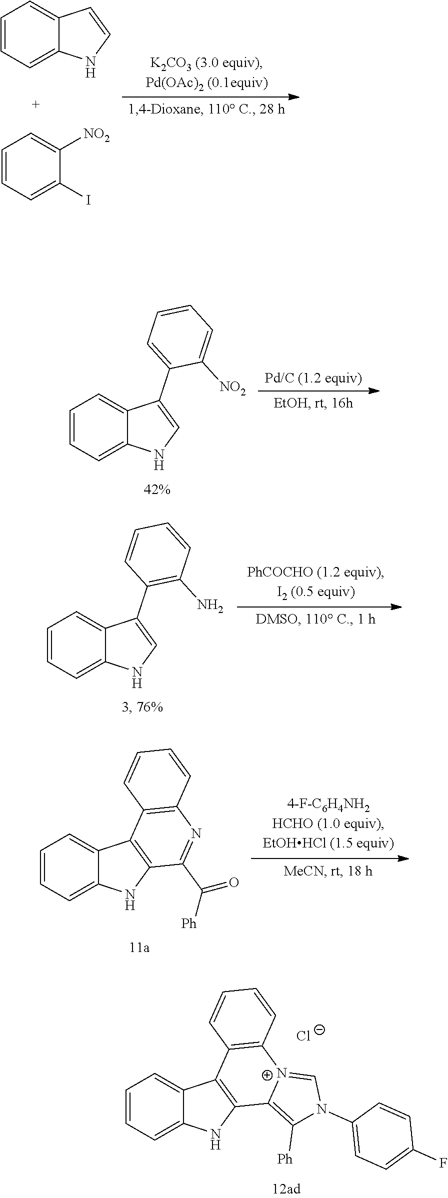

Compound 3 was prepared by the method known in the prior art (Batra et al, Eur. J. Org. Chem. 2009, 6211-6216). In 500 mL round bottom flask, solution of indole (5.0 g, 42.6 mmol) and o-iodonitrobenzene (12.7 μm, 51.2 mmol) in 1,4-dioxane (40 mL) was degassed with nitrogen for 15 min. K₂CO₃ (17.6 gm, 127.8 mmol) and Pd(OAc)₂ (0.95 gm, 4.26 mmol) were added to the above mixture under continuous flow of nitrogen. The resultant reaction mixture was heated at 110° C. for 28 h. After completion of reaction as monitored by TLC, the mixture was cooled to room temperature and filtered through the Celite, which was washed with EtOAc (100 mL×3). The combined filtrate was concentrated under reduced pressure. The crude product was purified by silica gel column chromatography using hexane/ethylacetate (90:10) as eluent to afford 3-(2-nitrophenyl)-1H-indole as a yellow solid (yield 4.3 g, 42%).

The compound 3-(2-nitrophenyl)-1H-indole (4.3 g, 18 mmol) was dissolved in ethanol (50 mL) and Pd—C (10%) (2.3 g, 21.6 mmol) was added portion wise under inert atmosphere. Resultant mixture was subjected to hydrogenation for 16 h using hydrogen balloon at room temperature. Thereafter, the reaction mixture was filtered through the Celite bed to remove the catalyst and washed with ethanol. The combined filtrate was evaporated in vacuum to obtain crude oil, which was purified by silica gel column chromatography using hexane/ethyl acetate (85:15, v/v) as eluent to obtain 2-(1H-indol-3-yl)aniline 3 as brown semisolid (yield 2.8 gm, 76%). 2-(1H-Indol-3-yl)aniline (3). Yield: 76% (2.8 g from 4.3 g); a yellow solid, R_{f}=0.69 (hexanes:EtOAc, 9:1, v/v); ¹H NMR (500 MHz, CDCl₃) δ (ppm)=8.42 (s, 1H), 7.72 (d, J=7.9 Hz, 1H), 7.42 (d, J=7.7 Hz, 2H), 7.34-7.23 (m, 3H), 6.96 (t, J=7.4 Hz, 1H), 6.91 (d, J=8.0 Hz, 1H), 3.90 (s, 2H) ppm; ¹³C NMR (125 MHz, CDCl₃) δ (ppm)=143.5, 135.1, 130.2, 126.9, 125.3, 122.0, 121.3, 119.57, 119.09, 118.89, 117.40, 114.41, 113.37, 110.37. MS (ESI+): m/z=209.4.

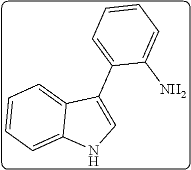 [SMILES: Nc1ccccc1-c1c[nH]c2ccccc12]

Step A. To a stirred solution of 2-(1H-indol-3-yl)aniline 3 (0.5 g, 2.4 mmol) in DMSO (6 mL), glyoxaldehyde (0.4 g, 2.9 mmol) was added portion wise and stirred at room temperature for 10 min. Iodine (0.3 g, 1.2 mmol) was added to the above mixture and the reaction was heated at 110° C. for 1 h. After complete consumption of amine (as evident by TLC) the mixture was cooled to room temperature and quenched with dilute Na₂S₂O₈ solution and extracted with ethyl acetate (25 mL×3). The organic layer was combined and washed with brine solution (25 mL), dried over anhydrous Na₂SO₄ and evaporated in vacuum to furnish the crude product. Purification via column chromatography over silica gel (60-120) using hexanes/EtOAc (95:5) as eluent gave compound 11a as a yellow solid (yield 0.65 gm, 84%). (7H-Indolo[2,3-c]quinolin-6-yl)(phenyl)methanone (11a). Yield: 84% (0.65 g from 0.5 g); a yellow solid, mp 198-200° C., R_{f}=0.68 (hexanes:EtOAc, 9:1, v/v); ¹H NMR (500 MHz, CDCl₃) δ (ppm) 10.61 (s, 1H), 8.75 (d, J=8.3 Hz, 1H), 8.59 (d, J=8.0 Hz, 1H), 8.50-8.48 (m, 2H), 8.36 (d, J=8.4 Hz, 1H), 7.82 (t, J=7.5 Hz, 1H), 7.73-7.68 (m, 2H), 7.66-7.60 (m, 2H), 7.58 (t, J=7.6 Hz, 2H), 7.45 (t, J=7.3 Hz, 1H).; ¹³C NMR (125 MHz, CDCl₃) δ (ppm) 195.2, 141.6, 139.76, 139.3, 137.1, 132.9, 132.7, 131.9, 131.9, 129.4, 128.1, 127.6, 126.2, 126.1, 123.9, 123.2, 123.1, 121.7, 121.1, 112.5. MS (ESI+): m/z=323.5 ESI-HRMS calculated for C₂₂H₁₄N₂O [M⁺+H]: 323.1179, found: 323.1169.

Step B. To a stirred solution of formaldehyde 37-41% solution in water (65 μL, 2.32 mmol) and 4-fluoroaniline (0.18 mL, 1.86 mmol) was added a freshly prepared ethanolic hydrochloride (0.5 mL) and the reaction was allowed to proceed for 20 min. A solution of 11 (0.50 g, 1.55 mmol) in acetonitrile (6 mL) was added and the reaction was continued stir for 18 h at room temperature. After completion of the reaction, as monitored by TLC, the solvent was evaporated under reduced pressure to afford the crude product, which was purified by column chromatography (MeOH/EtOAc. 1:9) to obtain compound 12 as a pale yellow solid. The product was recrystallized via triturating with diethyl ether/ethanol mixture to give pure product (0.4 g, 62%). 2-(4-Fluorophenyl)-1-phenyl-2,13-dihydroimidazo[1,5-a]indolo[2,3-c]quinolin-4-ium chloride (12ad). Yield: 62% (0.41 g from 0.5 g); a pale yellow solid, mp 323-326 C, R_{f}=0.37 (MeOH:EtOAc, 2:8, v/v); ¹H NMR (400 MHz, DMSO-d⁶) δ (ppm) 11.32 (s, 1H), 11.19 (s, 1H), 8.82 (q, J=7.8 Hz, 2H), 8.54 (d, J=8.0 Hz, 1H), 7.89 (t, J=7.6 Hz, 1H), 7.78 (t, J=7.8 Hz, 1H), 7.73-7.54 (m, 8H), 7.49-7.44 (m, 3H), 7.42-7.36 (m, 1H); ¹³C NMR (100 MHz, DMSO-d₆) δ (ppm) 139.5, 131.7, 131.4, 131.3, 130.4, 130.1, 130.0, 129.8, 127.0, 126.4, 125.7, 125.4, 125.1, 124.8, 123.5, 123.5, 122.6, 122.1, 121.3, 119.6, 118.0, 117.1, 116.9, 114.0, 110.3. MS (ESI): m/z=428.6. ESI-HRMS calculated for C₂₉H₁₉FN₃ [M⁺]: 428.1558, found: 428.1553.

All analogous compounds described below were prepared using identical experimental procedure.

(4-Bromophenyl)(7H-indolo[2,3-c]quinolin-6-yl)methanone (11b). Yield: 82% (0.79 g from 0.5 g); a yellow solid, mp: 227-230° C., R_{f}=0.72 (hexanes:EtOAc, 9:1, v/v); ¹H NMR (400 MHz, CDCl₃) δ (ppm) 10.60 (s, 1H), 8.76 (d, J=7.8 Hz, 1H), 8.60 (d, J=8.0 Hz, 1H), 8.41 (d, J=8.6 Hz, 2H), 8.35 (dd, J₁=8.4 Hz, J₁=0.6 Hz 1H), 7.90-7.82 (m, 1H), 7.74-7.70 (m, 4H), 7.70-7.62 (m, 1H), 7.50-7.45 (m, 1H); ¹³C NMR (125 MHz, CDCl₃) δ (ppm) 194.1, 141.6, 139.8, 138.9, 135.9, 133.4, 132.7, 131.9, 131.4, 129.6, 128.2, 127.7, 126.3, 126.2, 123.3, 123.2, 121.2, 112.5. MS (ESI+): m/z=402.2. ESI-HRMS calculated for C₂₂H₁₃BrN₂O [M⁺+H]: 403.0264, found: 403.0263.

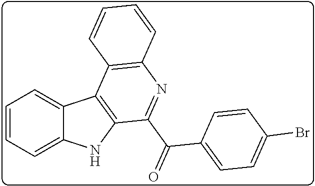 [SMILES: O=C(c1ccc(Br)cc1)c1nc2ccccc2c2c1[nH]c1ccccc12]

(4-Chlorophenyl)(7H-indolo[2,3-c]quinolin-6-yl)methanone (11c). Yield: 85% (0.73 g from 0.5 g); a yellow solid, mp: 226-229° C., R_{f}=0.68 (hexanes:EtOAc, 9:1, v/v); ¹H NMR (400 MHz, CDCl₃) δ (ppm) 10.60 (s, 1H), 8.76 (d, J=8.0 Hz, 1H), 8.60 (d, J=8.0 Hz, 1H), 8.50 (d, J=8.6 Hz, 1H), 8.35 (d, J=8.1 Hz, 1H), 7.90-7.82 (m, 1H), 7.74-7.70 (m, 2H), 7.64 (t, J=7.3 Hz, 1H), 7.55 (d, J=8.6 Hz, 2H), 7.47 (t, J=7.2 Hz, 1H); ¹³C NMR (125 MHz, CDCl₃) δ (ppm) 193.8, 135.4, 133.3, 132.6, 131.8, 129.5, 128.4, 127.7, 126.3, 126.2, 124.0, 123.2, 123.1, 121.7, 121.1, 112.5. MS (ESI+): m/z=357.6 (M⁺+H). ESI-HRMS calculated for C₂₂H₁₃ClN₂O [M⁺+H]: 357.0789, found: 357.0790.

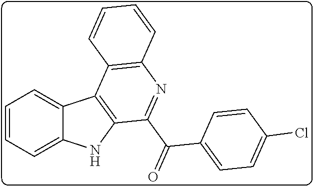 [SMILES: O=C(c1ccc(Cl)cc1)c1nc2ccccc2c2c1[nH]c1ccccc12]

(7H-Indolo[2,3-c]quinolin-6-yl)(p-tolyl)methanone (11d). Yield: 85% (0.68 g from 0.5 g); a ellow solid mp 164-167° C., R_{f}=0.69 (hexanes:EtOAc, 9:1, v/v); 1H NMR (400 MHz, CDCl₃) δ (ppm)=10.62 (s, 1H), 8.76 (d, J=8.3 Hz, 1H), 8.60 (d, J=8.1 Hz, 1H), 8.43 (d, J=8.1 Hz, 2H), 8.38 (d, J=8.4 Hz, 1H), 7.83 (t, J=7.5 Hz, 1H), 7.73-7.70 (m, 2H), 7.63 (t, J=7.6 Hz, 1H), 7.46 (t, J=7.5 Hz, 1H), 7.39 (d, J=8.1 Hz, 2H), 2.50 (s, 3H); ¹³C NMR (125 MHz, CDCl₃) δ (ppm)=194.7, 143.8, 141.6, 139.7, 139.6, 134.5, 132.7, 132.0, 131.8, 129.2, 128.8, 127.5, 126.1, 123.8, 123.2, 123.1, 121.7, 120.9, 112.4, 21.8. MS (ESI+): m/z=337.5. ESI-HRMS calculated for C₂₃H₁₆N₂O [M⁺]: 337.1335, found: 337.1319.

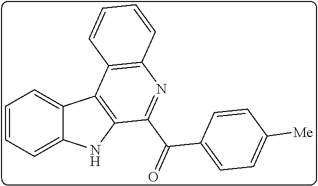 [SMILES: Cc1ccc(C(=O)c2nc3ccccc3c3c2[nH]c2ccccc23)cc1]

2-(4-Fluorophenyl)-1-(p-tolyl)-2,13-dihydrolmidazo[1,5-a]indolo[2,3-c]quinolin-4-ium chloride (12bc). Yield: 66% (0.44 g from 0.5 g); a pale yellow solid, mp above 300° C., R_{f}=0.37 (MeOH:EtOAc, 2:8, v/v); ¹H NMR (400 MHz, DMSO d⁶) δ (ppm) 11.26 (s, 1H), 11.17 (s, 1H), 8.84 (d, J=7.9 Hz, 1H), 8.77 (d, J=8.3 Hz, 1H), 8.54 (d, J=8.0 Hz, 1H), 7.89 (t, J=7.5 Hz, 1H), 7.80-7.72 (m, 2H), 7.70-7.66 (m, 2H), 7.50-7.33 (m, 9H), 7.30-7.24 (m, 1H), 2.43 (s, 3H); ¹³C NMR (100 MHz, (DMSO d⁶) δ (ppm) 140.9, 139.5, 131.5, 131.3, 130.7, 130.1, 130.1, 129.9, 127.0, 126.4, 125.9, 125.4, 125.1, 125.1, 124.9, 123.6, 123.3, 122.6, 122.1, 121.8, 121.3, 119.6, 117.9, 117.2, 116.9, 116.7, 114.1, 110.2, 21.7. MS (ESI+): m/z=442.5. ESI-HRMS calculated for C₃₀H₂₁FN₃ [M⁺]: 442.1714, found: 442.1712.

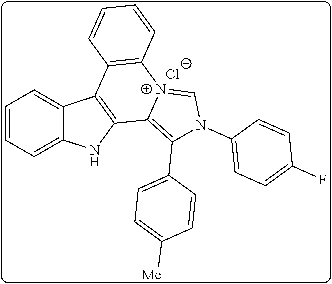 [SMILES: Cc1ccc(-c2c3c4[nH]c5ccccc5c4c4ccccc4[n+]3cn2-c2ccc(F)cc2)cc1 ; [Cl-]]

1-(4-Bromophenyl)-2-(4-chlorophenyl)-2,13-dihydroimidazo[1,5-a]indolo[2,3-c]quinolin-4-ium chloride (12bd). Yield: 65% (0.42 g from 0.5 g); a pale yellow solid, mp 249-253° C., R_{f}=0.39 (MeOH:EtOAc, 2:8, v/v); ¹H NMR (400 MHz, DMSO d⁶) δ (ppm) 11.29 (s, 1H), 8.85 (d, J=8.0 Hz, 1H), 8.77 (d, J=8.4 Hz, 1H), 8.55 (d, J=8.0 Hz, 1H), 7.90 (t, J=7.6 Hz, 1H), 7.83-7.70 (m, 5H), 7.64 (d, J=8.6 Hz, 2H), 7.51-7.37 (m, 4H).; ¹³C NMR (100 MHz, DMSO d⁶) δ (ppm) 139.6, 136.0, 133.7, 133.2, 132.8, 131.5, 130.2, 129.9, 129.4, 127.1, 126.3, 125.6, 125.3, 125.2, 124.3, 124.0, 123.3, 123.3, 122.6, 122.5, 121.5, 120.0, 117.9, 113.9, 110.5. MS (ESI+): m/z=524.4. ESI-HRMS calculated for C₂₉H₁₈BrClN₃ [M⁺]: 524.0347, found: 524.0331.

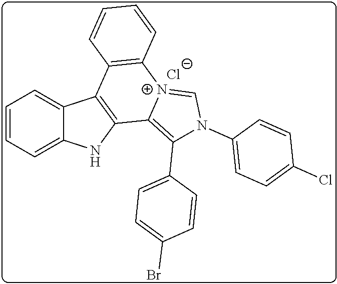 [SMILES: Clc1ccc(-n2c[n+]3c4ccccc4c4c5ccccc5[nH]c4c3c2-c2ccc(Br)cc2)cc1 ; [Cl-]]

1-4-Bromphenyl)-2-(4-fluorophenyl)-2,13-dihydroimidazo[1,5-a]indolo[2,3-c]quinolin-4-ium chloride (12cc). Yield: 63% (0.4 g from 0.5 g); a pale yellow solid, mp above 300° C., R_{f}=0.41 (MeOH:EtOAc, 2:8, v/v); ¹H NMR (400 MHz, DMSO d⁶) δ (ppm)=11.35 (s, 1H), 11.32 (s, 1H), 8.83 (q, J=8.4 Hz, 2H), 8.56 (d, J=8.1 Hz, 1H), 7.90 (t, J=7.6 Hz, 1H), 7.82 7.78 (m, 3H), 7.73-7.68 (m, 3H), 7.52-7.44 (m, 5H), 7.39 (t, J=7.5 Hz, 1H). ¹³C NMR (100 MHz, DMSO d⁶) δ (ppm)=139.6, 133.7, 133.2, 131.6, 130.2, 130.1, 129.9, 127.1, 126.4, 125.5, 125.3, 125.1, 124.5, 124.1, 123.3, 123.2, 122.6, 122.1, 121.4, 119.9, 118.1, 117.3, 117.0, 113.9, 110.4. MS (ESI+): m/z=506.4. ESI-HRMS calculated for C₂₉H₁₈BrFN₃ [M⁺]: 506.0663, found: 506.0664.

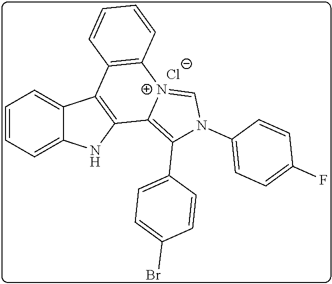 [SMILES: Fc1ccc(-n2c[n+]3c4ccccc4c4c5ccccc5[nH]c4c3c2-c2ccc(Br)cc2)cc1 ; [Cl-]]

2(4-Bromophenyl-1-(4-chlorophenyl)-2,13-dihydroimidazo[1,5-a]indolo[2,3-c]quinolin-4-ium chloride (12db). Yield, 61% (0.48 g from 0.5 g); a pale yellow solid, mp 330-335° C., R_{f}=0.50 (MeOH:EtOAc, 2:8, v/v); ¹H NMR (400 MHz, DMSO-d₆): δ (ppm) 11.30 (s, 2H), 8.85-8.76 (m, 2H), 8.55 (d, J=5.7 Hz, 1H), 7.87-7.68 (m, 8H), 7.57-7.39 (m, 6H).; ¹³C NMR (100 MHz, DMSO-d₆) δ (ppm) 139.6, 136.4, 133.5, 133.2, 131.4, 130.4, 129.9, 129.6, 127.1, 126.3, 125.6, 125.2, 124.7, 124.2, 123.6, 123.3, 122.6, 122.2, 121.5, 120.1, 117.9, 113.9, 110.5. MS (ESI+): m/z=522.1. ESI-HRMS calculated for C₂₉H₁₅BrN₃ [M⁺]: 522.0367, found: 522.0384.

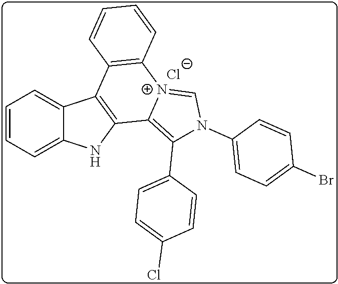 [SMILES: Clc1ccc(-c2c3c4[nH]c5ccccc5c4c4ccccc4[n+]3cn2-c2ccc(Br)cc2)cc1 ; [Cl-]]

1,2-Bis(4-chlorophenyl)-2,13-dihydroimidazo[1,5-a]indolo[2,3-c]quinolin-4-ium chloride (12dd). Yield: 64% (0.43 g from 0.5 g); a pale yellow solid, mp above 300° C., R_{f}=0.40 (MeOH:EtOAc, 2:8, v/v); ¹H NMR (400 MHz, DMSO d⁶) δ (ppm)=11.42 (s, 1H), 11.31 (s, 1H), 8.84 (t, J=7.4 Hz, 2H), 8.56 (d, J=7.8 Hz, 1H), 7.90-7.88 (m, 1H), 7.81-7.64 (m, 8H), 7.58 (d, J=7.8 Hz, 2H), 7.47-7.37 (m, 2H); ¹³C NMR (100 MHz, DMSO d⁶) δ (ppm)=139.6, 136.3, 135.9, 133.6, 132.8, 131.6, 130.3, 130.2, 129.9, 129.5, 127.1, 126.4, 125.5, 125.1, 124.3, 123.7, 123.3, 123.2, 122.6, 122.1, 121.4, 120.1, 118.1, 113.9, 110.5. MS (ESI+): m/z=478.5. ESI-HRMS calculated for C₂₉H₁₈Cl₂N₃ [M⁺]: 478.0872, found: 478.0868.

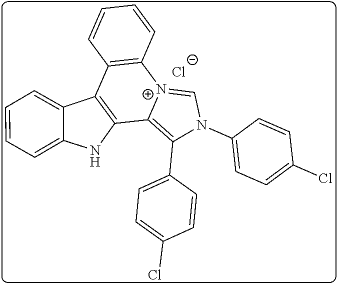 [SMILES: Clc1ccc(-c2c3c4[nH]c5ccccc5c4c4ccccc4[n+]3cn2-c2ccc(Cl)cc2)cc1 ; [Cl-]]

The compounds of the present invention can be used either alone or in combination with one or more therapeutic agents or pharmaceutically acceptable salts thereof. Such use will depend on the condition of the patient being treated and is well within the scope of a skilled practitioner.

Biological Activity: The compounds of general formula I have a stronger affinity towards KOR and modulate its property for potential therapeutic use.

### Agonist or Antagonist Activity of Synthesized Compounds at KOR, MOR and DOR

All compounds were evaluated at human KOR using GloSensor assay that measures the formation of cAMP in live cells expressing this receptor and luciferase-based cAMP biosensor (pGloSensof™-22F; Promega Corp. USA). As shown in Table 1, 41 compounds exhibited agonist activity at KOR with variable affinity (Log IC₅₀<−9 to −5). Notably, it was discovered that 14 compounds (8abc, 8abd, 8abe, 8acb, 8acc, 8acd, 8adb, 8adc, 8add, 8aec, 8aed 8ajc, 8akd, 8aod) act as agonist at KOR (pIC₅₀>7.5) with at least 100-fold selectivity over MOR and DOR. Given the adverse effect of KOR agonists such as aversion, sedation, lack of motor coordination and physchomimetic effects attributed to arrestinergic signaling, above mentioned 14 high affinity KOR agonists were evaluated for recruitment of arrestin to the activated receptor. To measure arrestin recruitment, β arrestin2-Tango assay was performed using HTLA cells transiently transfected with human KOR as described previously (Barnea G. et al, 2008, Proc Natl Acad Sci, USA, 105: 64-69). As shown in Table 2, 8 compounds (8abc, 8abd, 8acb, 8acc, 8acd, 8adc, 8add, 8ajc, 8akd) were discovered to exhibit greater than 100-fold potency towards G-protein dependent cAMP inhibition (GloSensor Assay) than recruitment of β-arrestin to the KOR. Thus, these compounds are suggested to be G-Protein biased KOR agonists.

### In Vivo Efficacy of Model Compound 8Abd

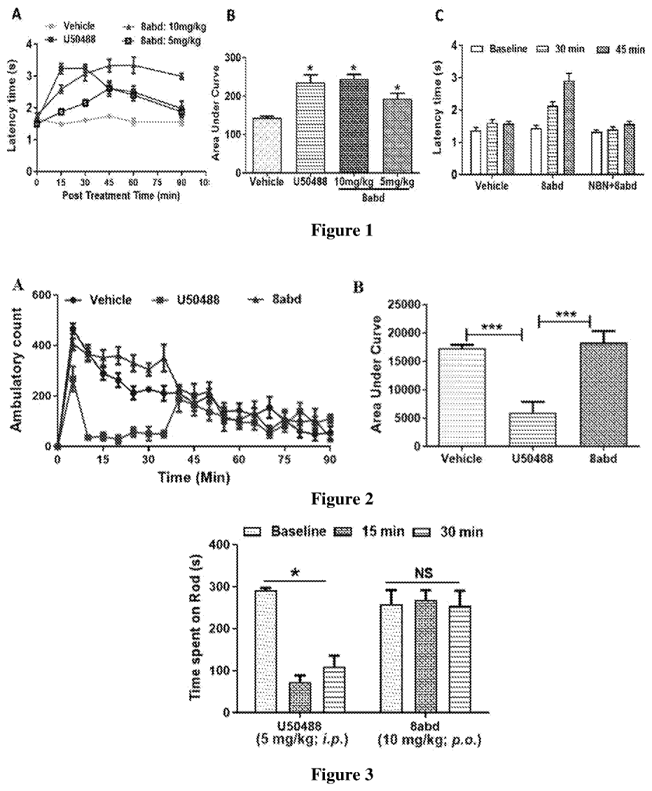

To determine if the highly biased agonist 8abd induces thermal analgesic response similar to other known agonists such as U50488. the effect of 8abd was tested in tail flick assay in C57Bl/6 mice following a preciously published protocol (Dogra et al. Sci Rep. 2016 Sep. 16; 6:33401). As shown in FIG. 1, this compound at 10 mg/kg or 5 mg/kg (p.o.) significantly increased tail flick latency time in comparison to vehicle treated mice. Also, the duration of the effect of this compound is comparable to U50488 (5 mg/kg, i.p.). Furthermore, the analgesic effect of this compound is completely blocked by selective KOR antagonist Norbinaltrophamine (NBN, 5 mg/kg, i.p.), clearly confirming specifically the engagement of KOR in vivo by this compound. [FIG. 1: Analgesic effect of 8abd in mice measured using tail flick assay (A) Time dependent analgesic effect of 8abd (5 and 10 mg/kg, p.o.) and U50488, (5 mg/kg, i.p.). Tail withdrawal latency was determined and presented as % baseline (tail flick measured at 15 min prior to vehicle or drug administration). (B) Bar graph showing area under the curve of tail flick test graph (*) p<0.01 by unpaired Student's t test (n=6-7 mice/group). (C) KOR selective antagonist norbinaltorphimine (NBN, 10 mg/kg, i.p.) blocked the analgesic effect of 8abd].

To determine if 8abd causes sedation or motor incoordination similar to a unbiased KOR agonist U50488, 8abd (10 mg/kg, p.o.) was administered in C57Bl/6 mice and performed open field test and rotarod test to measure sedative effect and latency to fall from rotating rod, respectively. As shown in FIGS. 2 and 3, 8abd does not induce any significant effect on both of these parameters. Whereas, unbiased and orthosteric KOR agonist U50488 induced marked sedation up to 30 minutes' post administration and also significantly decreased latency time to fall from the rotating rod. Thus. these results clearly suggest that due to G-Protein biased activity of 8abd, it lacks adverse effect profile associated with full and unbiased KOR agonists such as U50488 and others. [FIG. 2: Effect of 8abd on novelty induced locomotor in open field test. (A) Spontaneous locomotor activity for each 15-minute period. Total ambulatory counts were measured for 90 minutes after administration of vehicle or U50488 (5 mg/kg, i.p.) or 8abd (10 mg/kg, p.o.) using Opto-varimex 3 system. (B) Area under curve (shown in A) was calculated using Prizm Graphpad V software (*) p<0.01 by unpaired Student's t test (n=5-6 mice/group). FIG. 3: Effect of 8abd on motor coordination as evaluated by rotarod test. Treatment with U50488 (5 mg/kg; i.p.) significantly decreased the latency time to fall off the rotarod as compared to the baseline at 15 and 30 minutes after administration. Treatment with 8abd (10 mg/kg; p.o.) while did not effect the latency to fall. *p<0.05, ***p<0.01, one way ANOVA followed by Bonferroni post-hoc analysis (n=6 mice/group)].

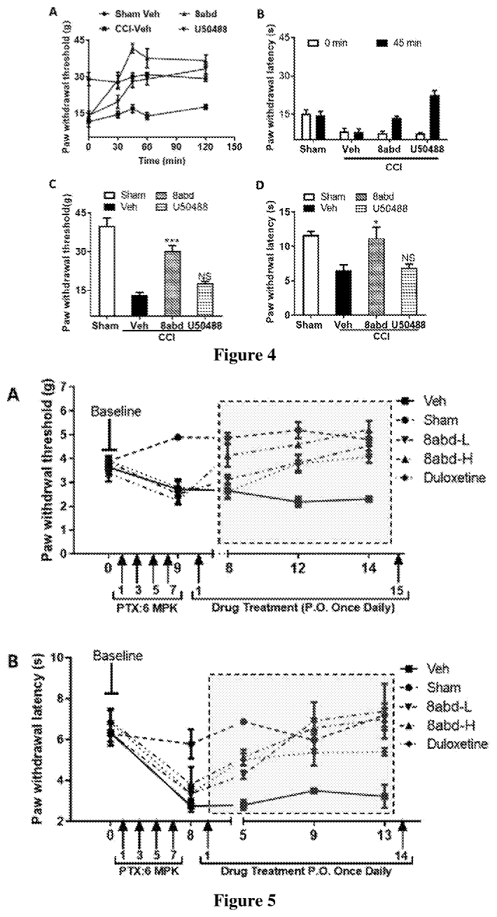

The acute and sub-chronic effects of model compound 8abd were also evaluated in rat model of neuropathic pain induced by chronic constriction injury (CCI) of sciatic nerve. As expected, acute administration model compound 8abd (10 mg/kg, p.o.) as well as U50488 (5 mg/kg. i.p.) robustly inhibited CCI-induced mechanical pain (FIG. 4A) and thermal hyperalgesia (FIG. 4B). Whereas, sub-chronic administration of only 8abd (10 mg/kg/day, 7 days, p.o.), not U50488 (5 mg/kg/day, 7 days, i.p.) significantly blocked the CCI-induced mechanical pain (FIG. 4C) and thermal hyperalgesia (FIG. 4D). These observations clearly highlight the therapeutic utility of G-protein biased agonist of KOR such as compound 8abd. [FIG. 4: Beneficial effect of 8abd on neuropathic pain (mechanical and thermal hyperalgesia) as evaluated by von frey test. (A, B) Both KOR agonists (8abd and U50488) attenuated allodynia (von frey hair threshold, A) and hyperalgesia (Hargreaves thermal pain, B) after acute administration in CCI model of neuropathic pain. (C, D) Subchronic treatment with 8abd (7 days, p.o.) attenuated both mechanical pain and hyperalgesia measured tested after 16 hours of drug administration, but subchronic treatment with U50488 did not attenuate CCI-induced pain and hyperalgesia. *p<0.05, ***p<0.01, one way ANOVA followed by Bonferroni post-hoc analysis (n=5-6 rats/group)].

Next, the chronic effect of 8abd was evaluated in the paclitaxel-induced peripheral neuropathy model in mice. Chronic administration of model compound 8abd (5 and 10 mg/kg, p.o.) and duloxetine (10 mg/kg, p.o.) alleviates mechanical pain (FIG. 5A) and thermal hyperalgesia (FIG. 5B). Compound 8abd shows better improvement in both pain behaviors as compared to duloxetine, a widely prescribed serotonin & norepinephrine reuptake inhibitor chronic pain. [FIG. 5: Chronic effect of 8abd on Paclitaxel-induced peripheral neuropathic pain. (A) Chronic treatment with 8abd (5 and 10 mg/kg, p.o.; 16 days) and Duloxetine (10 mg/kg, i.p.; 16 days) significantly improved the mechanical hyperalgesia in paclitaxel (PTX)-treated mice as measured by Von frey hair test (n=5 mice/group). (B) Chronic treatment with 8abd (5 and 10 mg/kg, p.o.; 16 days) and Duloxetine (10 mg/kg, i.p.; 16 days) significantly improved the thermal hyperalgesia in paclitaxel (PTX)-treated mice as measured by Hargreaves test (n=5 mice/group).]

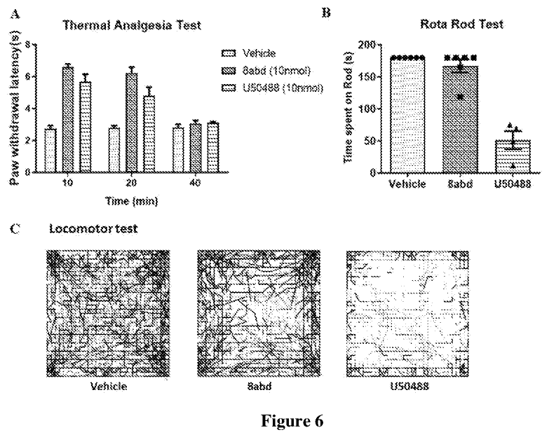

To determine if 8abd is mediating its effects via acting selectively in the brain, the behavioral effects of 8abd (10 nmol) and U50488 (10 nmol) after administrating directly into the brain ventricles using a cannula (FIG. 6) was determined. The Hargreaves assay for thermal analgesia, rotarod test for motor coordination and locomotor activity test for sedation was performed and it was found that 8abd exhibited analgesic activity without any sedation and motor incoordination. These data suggest that compound 8abd induces its beneficial effects via KOR expressed in the brain. [FIG. 6: Behavioral effects of intracerebroventricular (i.e.v.) of 8abd. (A) Time dependent analgesic effect of 8abd (10 nmol, i.e.v.) and U50488 (10 nmol, i.e.v.) was measured by paw withdrawal latency (n=5 mice/group). (B) Neuromuscular coordination was measured by Rotarod test after 10 minutes of 8abd administration (10 nmol, i.e.v.), mice spent more time on rod as compared to U50488 mice (n=5-6 mice/group). (C) Locomotor activity of 8abd (10nmol, i.e.v.) and U50488 (10nmol, i.e.v.) was measured in an open field test for 60 minutes after drug administration. The representive trajectory plot of the activity of the mice is shown, 8abd mice group showed locomotor activity without any sedation effect unlike U50488 group]

TABLE 1
Agonist activity of beta carboline
analogues at KOR, MOR and DOR.

| S. | Compound | | GloSensor Assay (Log IC₅₀) | | |
|---|---|---|---|---|---|
| No. | Codes | Molecular Formula | KOR | MOR | DOR |
| 1 | 8aaa | C₂₅H₁₈N₃Cl | >−5 | >−5 | >−5 |
| 2 | 8aab | C₂₅H₁₇BrClN₃ | −7.18 | >−5 | >−5 |
| 3 | 8aac | C₂₅H₁₇Cl₂N₃ | −6.9 | >−5 | >−5 |
| 4 | 8aad | C₂₅H₁₇ClFN₃ | −6.57 | >−5 | >−5 |
| 5 | 8aae | C₂₅H₁₇BrClN₃ | >−5 | >−5 | >−5 |
| 6 | 8aaf | C₂₅H₁₇Cl₂N₃ | >−5 | >−5 | >−5 |
| 7 | 8aba | C₂₅H₁₇BrClN₃ | −7.15 | >−5 | >−5 |
| 8 | 8abb | C₂₅H₁₆Br₂ClN₃ | >−5 | >−5 | >−5 |
| 9 | 8abc | C₂₅H₁₆BrCl₂N₃ | −9.2 | >−5 | >−5 |
| 10 | 8abd | C₂₅H₁₆BrClFN₃ | −8.3 | >−5 | >−5 |
| 11 | 8abe | C₂₅H₁₆Br₂ClN₃ | −8.9 | >−5 | >−5 |
| 12 | 8abf | C₂₅H₁₆BrCl₂N₃ | >−5 | >−5 | >−5 |
| 13 | 8abg | C₂₆H₁₉BrClN₃O | >−5 | >−5 | >−5 |
| 14 | 8abh | C₂₆H₁₆BrClF₃N₃O | >−5 | >−5 | >−5 |
| 15 | 8abi | C₂₅H₁₆BrClFN₃ | >−5 | >−5 | >−5 |
| 16 | 8abj | C₂₅H₁₆BrCl₂N₃ | −6.6 | −6.6 | >−5 |
| 17 | 8abk | C₂₅H₁₆BrClFN₃ | −7.1 | >−5 | >−5 |
| 18 | 8abl | C₂₅H₁₅BrCl₂FN₃ | >−5 | >−5 | >−5 |
| 19 | 8abm | C₂₅H₁₅BrClF₂N₃ | >−5 | >−5 | >−5 |
| 20 | 8aca | C₂₅H₁₆BrClFN₃ | −6.7 | >−5 | −5.5 |
| 21 | 8acb | C₂₅H₁₆Br₂ClN₃ | −8.6 | >−5 | >−5 |
| 22 | 8acc | C₂₅H₁₆BrCl₂N₃ | −8.8 | −5.7 | −5.4 |
| 23 | 8acd | C₂₅H₁₆BrClFN₃ | 8.8 | >−5 | >−5 |
| 24 | 8acf | C₂₅H₁₆BrCl₂N₃ | −7.2 | >−5 | >−5 |
| 25 | 8aci | C₂₅H₁₆BrClFN₃ | −7.2 | >−5 | >−5 |
| 26 | 8adb | C₂₅H₁₆BrCl₂N₃ | −7.7 | >−5 | >−5 |
| 27 | 8adc | C₂₅H₁₆Cl₃N₃ | −8.8 | >−5 | >−5 |
| 28 | 8add | C₂₅H₁₆Cl₂FN₃ | −8.4 | >−5 | >−5 |
| 29 | 8ade | C₂₅H₁₆BrCl₂N₃ | >−5 | >−5 | >−5 |
| 30 | 8adf | C₂₅H₁₆Cl₃N₃ | >−5 | >−5 | >−5 |
| 31 | 8adg | C₂₆H₁₉Cl₂N₃O | −6.1 | >−5 | >−5 |
| 32 | 8aea | C₂₅H₁₇Cl₂N₃ | >−5 | >−5 | >−5 |
| 33 | 8aeb | C₂₅H₁₆BrCl₂N₃ | >−5 | −5.9 | −5.7 |
| 34 | 8aec | C₂₅H₁₆Cl₃N₃ | −8.1 | −6.3 | >−5 |
| 35 | 8aed | C₂₅H₁₆Cl₂FN₃ | −8 | >−5 | >−5 |
| 36 | 8afc | C₂₅H₁₆Cl₃N₃ | −6.5 | >−5 | >−5 |
| 37 | 8afd | C₂₅H₁₆Cl₂FN₃ | −5.74 | >−5 | >−5 |
| 38 | 8aga | C₂₅H₁₇ClFN₃ | >−5 | >−5 | >−5 |
| 39 | 8agb | C₂₅H₁₆BrClFN₃ | >−5 | >−5 | >−5 |
| 40 | 8agc | C₂₅H₁₆Cl₂FN₃ | >−5 | >−5 | >−5 |
| 41 | 8agd | C₂₅H₁₆ClF₂N₃ | >−5 | >−5 | >−5 |
| 42 | 8age | C₂₅H₁₆BrClFN₃ | >−5 | >−5 | >−5 |
| 43 | 8agf | C₂₅H₁₆Cl₂FN₃ | >−5 | >−5 | >−5 |
| 44 | 8ahc | C₂₅H₁₆BrCl₂N₃ | >−5 | 6.8 | >−5 |
| 45 | 8aic | C₂₅H₁₆Cl₂FN₃ | −7.04 | >−5 | >−5 |
| 46 | 8aid | C₂₅H₁₆ClF₂N₃ | −6.7 | >−5 | >−5 |
| 47 | 8ajc | C₂₅H₁₆Cl₂FN₃ | −8 | >−5 | >−5 |
| 48 | 8akd | C₂₆H₁₉ClFN₃ | −8.8 | >−5 | >−5 |
| 49 | 8ald | C₂₅H₁₆ClFN₄O₂ | >−5 | >−5 | >−5 |
| 50 | 8amd | C₂₇H₁₈ClN₃O | −7.4 | >−5 | >−5 |
| 51 | 8and | C₂₃H₁₅ClFN₃S | −6.68 | >−5 | >−5 |
| 52 | 8aod | C₂₇H₂₁Cl₂N₃O₂ | −7.9 | >−5 | >−5 |
| 53 | 8apd | C₂₈H₂₃ClFN₃O₃ | >−5 | >−5 | >−5 |
| 54 | 8bbc | C₂₇H₁₈BrCl₂N₃O₂ | >−5 | >−5 | >−5 |
| 55 | 8bbd | C₂₇H₁₈BrCl₂FN₃O₂ | >−5 | >−5 | >−5 |
| 56 | 8bkd | C₂₈H₂₁ClFN₃O₂ | >−5 | >−5 | >−5 |
| 57 | 8bpd | C₂₂H₁₇ClFN₃O₂ | −5.5 | >−5 | >−5 |
| 58 | 8cca | C₂₅H₁₆Br₂ClN₃ | >−5 | >−5 | >−5 |
| 59 | 8dbc | C₂₆H₁₈BrCl₂N₃ | >−5 | >−5 | >−5 |
| 60 | 8dbd | C₂₆H₁₈BrClFN₃ | >−5 | >−5 | >−5 |
| 61 | 8ddc | C₂₆H₁₈Cl₃N₃ | >−5 | >−5 | >−5 |
| 62 | 9aaa | C₂₆H₁₉BrClN₃ | >−5 | >−5 | >−5 |
| 63 | 9aab | C₂₆H₁₈BrClFN₃ | −6.58 | >−5 | >−5 |
| 64 | 9aac | C₃OH₂₇BrClN₃ | >−5 | >−5 | >−5 |
| 65 | 9aad | C₂₆H₁₇BrCl₂FN₃ | >−5 | >−5 | >−5 |
| 66 | 9aae | C₂₇H₂₁BrClN₃O | −5.48 | >−5 | >−5 |
| 67 | 9aaf | C₂₆H₁₇BrClF₂N₃ | >−5 | >−5 | >−5 |
| 68 | 9aag | C₂₈H₂₃BrClN₃O₂ | −6.78 | >−5 | >−5 |
| 69 | 9abe | C₂₇H₂₁Cl₂N₃O | −7.12 | >−5 | >−5 |
| 70 | 9abg | C₂₈H₂₃Cl₂N3O₂ | −6.02 | >−5 | >−5 |
| 71 | 9abh | C₂₇H₁₈Cl₂F₃N₃O | >−5 | >−5 | >−5 |
| 72 | 9acj | C₂₄H₁₇Cl₂N₃S | >−5 | >−5 | >−5 |
| 73 | 12ad | C₂₉H₁₉ClFN₃ | >−5 | >−5 | >−5 |
| 74 | 12bc | C₃₀H₂₁ClFN₃ | >−5 | >−5 | >−5 |
| 75 | 12bd | C₂₉H₁₈BrCl₂N₃ | >−5 | >−5 | >−5 |
| 76 | 12cc | C₂₉H₁₈BrClFN₃ | >−5 | >−5 | >−5 |
| 77 | 12db | C₂₉H₁₈BrClN₃ | −6.6 | ND | ND |
| 78 | 12dd | C₂₉H₁₈Cl₃N₃ | >−5 | >−5 | >−5 |

TABLE 2
Compounds exhibiting G-protein bias in
comparison to arrestinergic signaling

| | GloSensor | | G-Protein Bias factor |
|---|---|---|---|
| Compound | Assay | B-arrestin Tango | (IC₅₀ -Glosensor/- EC₅₀ - |
| ID | (pIC50) | Assay (pEC50) | Tango) |
| 8abc | 8.95 | 6.6 | 2.35 |
| 8abd | 8.98 | 6.65 | 2.33 |
| 8abe | 7.89 | 6.17 | 1.72 |
| 8acb | 9.94 | 6.34 | 3.6 |
| 8acc | 9.23 | 6.34 | 2.89 |
| 8acd | 8.8 | 5.8 | 3 |
| 8adb | 7.7 | 6.26 | 1.44 |
| 8adc | 9.30 | 6.02 | 3.28 |
| 8add | 8.532 | 5.86 | 2.67 |
| 8aec | 8.1 | 6.87 | 1.23 |
| 8aed | 8.0 | 6.94 | 1.06 |
| 8ajc | 8.002 | 5.385 | 2.61 |
| 8akd | 9.193 | 6.038 | 3.15 |
| 8aod | 7.9 | 6.22 | 1.68 |
| U50488 | 9.3 | 8.51 | 0.79 |

### Biological Methods

### GloSensor Assay:

GloSensor assay was performed to measure KOR-induced (Gᵢ-mediated) cAMP inhibition following the protocol as described previously (Kumar B A et al, Methods Cell Biol. 2017; 142: 27-50). Briefly, HEK-293T cells were co-transfected with human KOR and luciferase based cAMP biosensor (pGloSensor™-22F plasmid; Promega) using PEImax method of transfection in the ratio of 1:4. The transfected cells were incubated at 37° C. in tissue culture incubator with 95% O₂/5% CO₂ for 14-16 hours. After incubation, media from the plate was aspirated and 100 μl of sodium luciferin solution (1 mg/ml) was added to the cell plates. The plates were then incubated at 37° C. in a tissue culture incubator with 95% O₂/5% CO₂ for 90 minutes. After incubation, cells were treated with 20 μl of 6× diluted standard U50488 (10000 nM to 0.01 nM) along with test compounds (in triplicates) and were incubated in a humidified tissue culture incubator at 37° C. with 95% O₂/5% CO₂ for 10-15 minutes to get a steady state condition. Ligand incubation was followed by addition of 20 μl of 70 μM forskoline (FSK) to the plate. FSK incubation was followed by incubation at 37° C. for 10-15 minutes and measurement of luminescence per well using luminescence plate reader (BMG Labtech). The results were plotted as inhibition of FSK-stimulated cAMP response (relative luminescence units) using nonlinear regression analysis by Graph-Pad prism.

### β-Arrestin Tango Assay

β-arrestin recruitment was measured by previously described “Tango” assay method (Dogra S. et al, Methods Cell Biol. 2016; 132, 233-54). Tango assay is carried out in HEK293T cells genetically modified to express human β-arrestin2 fused with tobacco etch virus (TEV) protease and a tetracycline-controlled transactivator (tTA)-driven luciferase reporter gene. These cells were transiently transfected with Tango-compatible human kappa opioid receptor cDNA. Briefly, HTLA cells (kind gift from Dr. Gilad Barnea, Brown University, USA) were transiently transfected with human KOR-Tango receptor plasmid DNA (Kind gift from Dr. Bryan Roth, UNC-Chapel Hill, USA) and plated in 96 well tissue culture plate with high glucose media containing 5% fetal bovine serum. Transfected cells were incubated at 37° C. with 5% CO₂ in a humidified incubator for six hours. After six hours incubation, cells were treated with test ligands at different concentrations and further incubated for overnight. Finally, media from treated cell culture plate were discarded and 100 mL of sodium luciferin substrate (1 mg/ml) to each well of 96 well plates were added. After substrate addition luminescence was measured using multimode plate reader (BMG, Labtech) and normalized luminescence data were analyzed using GraphPad Prism 5.0 to calculate EC₅₀/IC₅₀.

### Behavioral Tests:

All animal experiments and procedures were executed in accordance with the guidelines established in the guide for the care and use of laboratory animals and were approved by the institutional animal ethics committee (IAEC) of CSIR-Central Drug Research Institute, Lucknow, India. In this study, male C57BL/6 mice (6-8 weeks old) weighing 22-25 g and Sprague Dawley (SD) rats weighing 200-250 g were used. Animals were housed on a 12-h light/dark cycle (lights on at 8.00 am) and food and water were provided ad libitum. Animals were acclimatized to the experimental room 30 minutes prior to the beginning of behavioral procedures.

Locomotor activity: Measurement of locomotor activity in normal C57BIU6 mice were performed using either Opto-varimex 3 system (Columbus Instruments, OH, USA) or, which is an advanced system using the advance technology to quantify locomotor activity and trace the animal's path for behavioral analysis. This system senses motion with a grid of infra red photocells placed around the arena (17.5″×17.5), and provides total, ambulatory and vertical counts of mice locomotor activity in this open arena. Briefly, mice were administrated with either vehicle (0.9% normal saline) or U50488 or 8abd and mice were placed to the centre of a clear Plexiglas (40×40×30 cm) open-field arena to let them explore the entire arena for 90 minutes. The animals were removed from the arena and the whole arenas were cleaned with water and smell-free detergent solution to remove the olfactory cues of previous animals associated with the chamber.

Measurement of locomotor activity in I.C.V. cannulated mice was performed using Rodent Locomotor activity test system (Panlab, Harvard Apparatus). Briefly, mice were administered with either ACSF or 8abd (10 nmol) or U50488 (10 nmol) directly in the right ventricle using guide cannula and placed in the centre of the arena (45×45×30 cm) to let them explore the arena for 60 minutes. The trajectory plot of the activity was measured using ACTITRACK software provided by manufacturer.

Tail flick test for analgesia: This test is used to access the pain response in small laboratory animals. By observing the reaction of animal's tail to heat, it is used to measure the effectiveness of analgesics. It is based upon the fact that the tail skin temperature plays an important role in the critical temperature (the temperature at which the tail flicks in response to pain). This test was done in C57Bl/6 as described previously with minor modifications (Ravilla L et al, J Med Chem. 2017 Aug. 10; 60(15):6733-6750). Specifically, hot water bath set at 52°±0.5° C. was used as a heat source and mice were individually acclimatized to the restrainers for one minute without tail immersion. After acclimatization, each mouse was gently introduced into the restrainers and the distal one-third of the tail was dipped into the hot water bath aet at 52°±0.5° C. and tail withdrawal latencies were recorded thrice at a time point by an observer blinded to the identity of the animal. Latency time recorded in three trials was averaged and presented as mean±SEM. For every mouse, cut-off time was set at 15 seconds to avoid heat-mediated damage to tail.

Rotarod test for motor coordination: Balance and motor coordination were measured using a fixed speed (20 rpm) rotarod after drug treatment (Rotamex-4/8 system, Columbus Instruments) as previously reported (Huang H S et al, Behav Brain Res. 2013; 243:79-90). Briefly, mice were trained on the rotarod apparatus for two trials/day at 5 rpm, 10 rpm and finally at 20 rpm, with a 5-minute inter trial intervals. Once the mice were trained to stay on rotating rod for 5 minutes, the effect of drugs on motor coordination was evaluated. On testing day, each mouse first completed a drug-free trial to determine baseline performance before drug administration. After measuring baseline performance, mice were injected with test compounds and were placed on the rotating rod (20 rpm) 15 minutes after drug administration. The latency to fall off the rod was measured by the rotarod timer over a period of 5-minutes. Additionally, the testing was stopped for mice that rotated off the top of the rod.

Chronic Constriction Injury (CCI) of sciatic nerve in SD rats: Neuropathic pain was induced by chronic constriction injury (CCI) of the sciatic nerve according to previously described model (Bennett and Xie, Pain. 1988 April; 33(1):87-107). Briefly, SD rats were anesthetized with ketamine and zylazine (90 and 10 mg/kg, i.p., respectively), and the left sciatic nerve was exposed and 3 loose ligatures with 5-0 silk suture thread were made on the nerve at 1.0-1.5 mm intervals. The muscle and skin were sutured after complete hemostasis. The rats were divided into 4 groups according to the ligation/medicinal intervention as follows: The sham (Con) (n=5), CCI (n=5), CCI+U50488 (n=5, 5 mg/kg, i.p.), CCI+8abd (n=5, 10 mg/kg, p.o.). In the Sham group, the sciatic nerve was exposed but without ligation; the rats in all the other groups underwent both exposure and ligation of the left sciatic nerve.

Chemotherapy-induced Peripheral Neuropathic Pain (CIPN): The paclitaxel-induced peripheral neuropathy was developed as describes previously (Polomono et al., Pain. 2001; 94:293-304). In this assay, 8-10 weeks old SD rats were injected with paclitaxel (Taxol; 6 mg/kg, i.p.) every alternate day for a total of four injections (1, 3, 5 and 7 days), resulting in a cumulative dose of 24 mg/kg. The control animals received an equal volume of saline solution. Mechanical and thermal withdrawal thresholds were tested before, during and after paclitaxel treatment.

Mechanical and thermal hyperalgesia assessment: The presence of mechanical hypersensitivity was accessed by measuring paw withdrawal threshold using electronic von frey anesthesiometer (800 gm rigid tips, IITC Life Science Inc., CA, USA) as described previously (Yalcin et al, Ann Neurol. 2009 February; 65(2):218-25). Briefly, the animals were placed individually in transparent Perspex boxes with wire mesh walls and floor for 20 min of habituation time prior to behavioral tests. The filaments were individually applied vertically to the plantar side of the right hind paw and repeated 3 times at intertrial interval of 5 minutes at each time point per paw. A positive response was defined as the minimal force that caused at least 2 withdrawals observed out of 3 consecutive trials. A maximal cut-off value of 60 g was used to prevent tissue damage.

Thermal hyperalgesia was measured as previously described (Hargreaves et al, Pain. 1988 January; 32(1):77-88). The animals were placed on a glass plate surrounded by Plexiglas to acclimate to the device (IITC Life Science Inc., CA, USA). The temperature was increased by a heat source under the plantar surface of the hind paw. Clear paw withdrawal, shaking and/or licking were considered nociceptive-like responses. The nociceptive threshold was recorded in seconds and repeated 3 times at 5-min intervals. A cut-off time of 30 sec was used to avoid tissue damage.

### Intracerebroventricular (ICV) Cannula Installation for Drug Delivery:

C57BL/6 mice were anesthetized with ketamine and xylazine (90 mg/kg and 10 mg/kg, respectively) and were positioned in a stereotaxic frame (Stoelting Co.). Briefly, an incision was made in the skin and muscle atop the skull was pulled to the side and cleaned. A craniotomy was made following the stereotaxic coordinates (in mm relative to Bregma): AP −0.5, ML±1.1. A 2.2 mm deep 26-gauge intracerebroventricular cannula (Plastic One, Roanoke, USA) reaching the right ventricle was installed over the skull using screws followed by application of dental cement. Once the dental cement was hardened, the remaining skin was adhered using a tissue adhesive (Vetbond, 3M Science, USA). The animals were further allowed to acclimate with implanted cannula for 1 week during which they were given ibuprofen (0.04 mg/ml of drinking water) and gentamycin (1 mg/kg i,p) for first three days to alleviate pain and to avoid infection.

We claim:

1. A compound of formula I 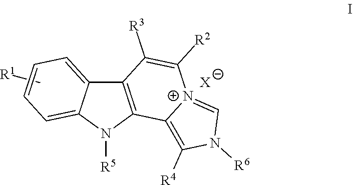
tautomer, stereoisomer or pharmaceutically acceptable salts thereof, wherein:
R¹ is selected from hydrogen, halogen, alkoxy, benzyloxy, sulphonic acid, sulphonamide, cyano, nitro or amide;
R² is selected from hydrogen, C₁-2 alkyl, alkyloycarbonyl or aryloxycarbonyl;
R³ is selected from hydrogen or aryl;
R² and R³ may jointly form an aromatic ring having substituent selected from hydrogen, trifluoromethyl, nitro or halogen;
R⁴ is selected from substituted or unsubstituted alkyl, aryl or heteroaryl, wherein the substitutents are selected from the groups consisting of halogen, alkyl, aryl, alkoxy, aryloxy, thiomethyl, trifluoromethyl, trifluoromethyloxy, thioalkyl, sulphonylamide and nitro;
R⁵ is selected from H, alkyl, alkenyl, cycloalkyl aryl, arylalkyl, heteroaryl, benzoyl, sulphonylphenyl, or alkoxycarbonyl;
R⁶ is selected from substituted or unsubstituted alkyl, aryl, arylalkyl, 5 or 6-membered heteroaryl, alkoxycarbonylalkyl, cycloalkyl, or hetercycloalkyl, wherein the substitutents are selected from halogen, alkyl, alkoxy, aryl or alkyl aminoalkyl; and
X is selected from Cl, I, ClO₄, CF₃SO₂, BF₄ or PF₆.

2. The compound as claimed in claim 1, wherein R¹, R², R³ and R⁵ are H; R⁴ is selected from aryl, heteroaryl optionally substituted with halogen, alkyl, alkoxy or nitro and R⁶ is selected from alkyl, aryl, arylalkyl, 5 or 6-membered heteroaryl optionally substituted with halogen, alkyl, alkoxy or aryl.

3. The compound as claimed in claim 1, wherein R¹ and R⁵ are H; R² and R³ jointly form a aromatic ring, R⁴ is selected from aryl, heteroaryl optionally substituted with halogen, alkyl, alkoxy or nitro and R⁶ is alkyl, aryl, arylalkyl, 5 or 6-membered heteroaryl optionally substituted with halogen, alkyl, alkoxy, or aryl.

4. The compound as claimed in claim 1, wherein the compound is selected from the group consisting of:
1,2-Diphenyl-2,11-dihydroimidazo[1′,5′:1,2]pyrido[3,4-b]indol-4-ium chloride (8aaa), 2-(4-Bromophenyl)-1-phenyl-2,11-dihydroimidazo[1′,5′:1,2]pyrido[3,4-b]indol-4-ium chloride (8aab), 2-(4-Chlorophenyl)-1-phenyl-2,11-dihydroimidazo[1′,5′:1,2]pyrido[3,4-b]indol-4-ium chloride (8aac), 2-(4-Fluorophenyl)-1-phenyl-2,11-dihydroimidazo[1′,5′:1,2]pyrido[3,4-b]indol-4-ium chloride (8aad), 2-(3-Bromophenyl)-1-phenyl-2,11-dihydroimidazo[1′,5′:1,2]pyrido[3,4-b]indol-4-ium chloride (8aae), 2-(3-Chlorophenyl)-1-phenyl-2,11-dihydroimidazo[1′,5′:1,2]pyrido[3,4-b]indol-4-ium chloride (8aaf), 1-(4-Bromophenyl)-2-phenyl-2,11-dihydroimidazo[1′,5′:1,2]pyrido[3,4-b]indol-4-ium chloride (8aba), 1,2-Bis(4-bromophenyl)-2,11-dihydroimidazo[1′,5′:1,2]pyrido[3,4-b]indol-4-ium chloride (8abb), 1-(4-Bromophenyl)-2-(4-chlorophenyl)-2,11-dihydroimidazo[1′,5′:1,2]pyrido[3,4-b]indol-4-ium chloride (8abc), 1-(4-Bromophenyl)-2-(4-fluorophenyl)-2,11-dihydroimidazo[1′,5′:1,2]pyrido[3,4-b]indol-4-ium chloride (8abd), 2-(3-Bromophenyl)-1-(4-bromophenyl)-2,11-dihydroimidazo[1′,5′:1,2]pyrido[3,4-b]indol-4-ium chloride (8abe), 1-(4-Bromophenyl)-2-(3-chlorophenyl)-2,11-dihydroimidazo[1′,5′:1,2]pyrido[3,4-b]indol-4-ium chloride (8abf), 1-(4-Bromophenyl)-2-(4-methoxyphenyl)-2,11-dihydroimidazo[1′,5′:1,2]pyrido[3,4-b]indol-4-ium chloride (8abg), 1-(4-Bromophenyl)-2-(4-(trifluoromethoxy)benzyl)-2,11dihydroimidazo[1′,5′:1,2]pyrido[3,4-b]indol-4-ium chloride (8abh), 1-(4-Bromophenyl)-2-(4-5′:1,2]pyrido[3,4-b]indol-4-ium chloride (5abh), 1-(4-Bromophenyl)-2-(3-fluorophenyl)-2,11-dihydroimidazo[1′,5′:1,2]pyrido[3,4-b]indol-4-ium chloride (8abi), 1-(4-Bromophenyl)-2-(2-chlorophenyl)-2,11-dihydroimidazo[1′,5′:1,2]pyrido[3,4-b]indol-4-ium (8abj), 1-(4-Bromophenyl)-2-(2-fluorophenyl)-2,11-dihydroimidazo[1′,5′:1,2]pyrido[3,4-b]indol-4-ium chloride (8abk), 1-(4-Bromophenyl)-2-(2-chloro-4-fluorophenyl)-2,11-dihydroimidazo[1′,5′:1,2]pyrido[3,4-b]indol-4-ium chloride (8abl), 1-(4-Bromophenyl)-2-(3,4-difluorophenyl)-2,11-dihydroimidazo[1′,5′:1,2]pyrido[3,4-b]indol-4-ium chloride (8abm), 1-(2-Bromophenyl)-2-phenyl-2,11-dihydroimidazo[1′,5′:1,2]pyrido[3,4-b]indol-4-ium chloride (8aca), 1-(2-Bromophenyl)-2-(4-bromophenyl)-2,11-dihydroimidazo[1′,5′:1,2]pyrido[3,4-b]indol-4-ium chloride (8acb), 1-(2-Bromophenyl)-2-(4-chlorophenyl)-2,11-dihydroimidazo[1′,5′:1,2]pyrido[3,4-b]indol-4-ium (8acc), 1-(2-Bromophenyl)-2-(4-fluorophenyl)-2,11-dihydroimidazo[1′,5′:1,2]pyrido[3,4-b]indol-4-ium chloride (8acd), 1-(2-Bromophenyl)-2-(3-chlorophenyl)-2,11-dihydroimidazo[1′,5′:1,2]pyrido[3,4-b]indol-4-ium chloride (8acf), 1-(2-Bromophenyl)-2-(3-fluorophenyl)-2,11-dihydroimidazo[1′,5′:1,2]pyrido[3,4-b]indol-4-ium (8aci), 2-(4-Bromophenyl)-1-(4-chlorophenyl)-2,11-dihydroimidazo[1′,5′:1,2]pyrido[3,4-b]indol-4-ium chloride (8adb), 1,2-Bis(4-chlorophenyl)-2,11-dihydroimidazo[1′,5′:1,2]pyrido[3,4-b]indol-4-ium chloride (8adc), 1-(4-Chlorophenyl)-2-(4-fluorophenyl)-2,11-dihydroimidazo[1′,5′:1,2]pyrido[3,4-b]indol-4-ium chloride (8add), 2-(3-Bromophenyl)-1-(4-chlorophenyl)-2,11-dihydroimidazo[1′,5′:1,2]pyrido[3,4-b]indol-4-ium chloride (8ade), 2-(3-Chlorophenyl)-1-(4-chlorophenyl)-2,11-dihydroimidazo[1′,5′:1,2]pyrido[3,4-b]indol-4-ium chloride (8adf), 1-(4-Chlorophenyl)-2-(4-methoxyphenyl)-2,11-dihydroimidazo[1′,5′:1,2]pyrido[3,4-b]indol-4-ium chloride (8adg), 1-(2-Chlorophenyl)-2-phenyl-2,11-dihydroimidazo[1′,5′:1,2]pyrido[3,4-b]indol-4-ium chloride (8aea), 2-(4-Bromophenyl)-1-(2-chlorophenyl)-2,11-dihydroimidazo[1′,5′:1,2]pyrido[3,4-b]indol-4-ium chloride (8aeb), 1-(2-Chlorophenyl)-2-(4-chlorophenyl)-2,11-dihydroimidazo[1′,5′:1,2]pyrido[3,4-b]indol-4-ium chloride (8aec), 1-(2-Chlorophenyl)-2-(4-fluorophenyl)-2,11-dihydroimidazo[1′,5′:1,2]pyrido[3,4-b]indol-4-ium chloride (8aed), 1-(3-Chlorophenyl)-2-(4-chlorophenyl)-2,11-dihydroimidazo[1′,5′:1,2]pyrido[3,4-b]indol-4-ium chloride (8afc), 1-(3-chlorophenyl)-2-(4-fluorophenyl)-2,11-dihydroimidazo[1′,5′:1,2]pyrido[3,4-b]indol-4-ium chloride (8afd), 1-(4-Fluorophenyl)-2-phenyl-2,11-dihydroimidazo[1′,5′:1,2]pyrido[3,4-b]indol-4-ium chloride (8aga), 2-(4-Bromophenyl)-1-(4-fluorophenyl)-2,11-dihydroimidazo[1′,5′:1,2]pyrido[3,4-b]indol-4-ium chloride (8agb), 2-(4-Chlorophenyl)-1-(4-fluorophenyl)-2,11-dihydroimidazo[1′,5′:1,2]pyrido[3,4-b]indol-4-ium chloride (8agc), 1,2-Bis(4-fluorophenyl)-2,11-dihydroimidazo[1′,5′:1,2]pyrido[3,4-b]indol-4-ium chloride (8agd), 2-(3-Bromophenyl)-1-(4-fluorophenyl)-2,11-dihydroimidazo[1′,5′:1,2]pyrido[3,4-b]indol-4-ium chloride (Sage), 2-(3-Chlorophenyl)-1-(4-fluorophenyl)-2,11-dihydroimidazo[1′,5′:1,2]pyrido[3,4-b]indol-4-ium chloride (8agf), 1-(4-Bromophenyl)-2-(4-chlorophenyl)-2,11-dihydroimidazo[1′,5′:1,2]pyrido[3,4-b]indol-4-ium (8ahc), 2-(4-Chlorophenyl)-1-(3-fluorophenyl)-2,11-dihydroimidazo[1′,5′:1,2]pyrido[3,4-b]indol-4-ium chloride (8aic), 1-(3-Fluorophenyl)-2-(4-fluorophenyl)-2,11-dihydroimidazo[1′,5′:1,2]pyrido[3,4-b]indol-4-ium (8aid), 1-(2-Bromo-4-fluorophenyl)-2-(4-chlorophenyl)-2,11-dihydroimidazo[1′,5′:1,2]pyrido[3,4-b]indol-4-ium (8ajc), 2-(4-Fluorophenyl)-1-(p-tolyl)-2,11-dihydroimidazo[1′,5′:1,2]pyrido[3,4-b]indol-4-ium chloride (8akd), 2-(4-Fluorophenyl)-1-(4-nitrophenyl)-2,11-dihydroimidazo[1′,5′:1,2]pyrido[3,4-b]indol-4-ium chloride (8ald), 1-(Benzofuran-2-yl)-2-phenyl-2,11-dihydroimidazo[1′,5′:1,2]pyrido[3,4-b]indol-4-ium chloride (8amd), 2-(4-Fluorophenyl)-1-(thiophen-2-yl)-2,11-dihydroimidazo[1′,5′:1,2]pyrido[3,4-b]indol-4-ium chloride (Sand), 2-(4-Fluorophenyl)-1-(4-methoxyphenyl)-2,11-dihydroimidazo[1′,5′:1,2]pyrido[3,4-b]indol-4-ium (8aod), 2-(4-fluorophenyl)-1-(3,4,5-trimethoxyphenyl)-2,11-dihydroimidazo[1′,5′:1,2]pyrido[3,4-b]indol-4-ium chloride (8apd), 1-(4-Bromophenyl)-2-(4-chlorophenyl)-5-(methoxycarbonyl)-2,11-dihydroimidazo[1′,5′:1,2]pyrido[3,4-b]indol-4-iumchloride (8bbc), 1-(4-Bromophenyl)-2-(4-fluorophenyl)-5-(methoxycarbonyl)-2,11-dihydroimidazo[1′,5′:1,2]pyrido[3,4-b]indol-4-iumchloride (8bbd), 2-(4-Fluorophenyl)-5-(methoxycarbonyl)-1-(p-tolyl)-2,11-dihydroimidazo[1′,5′:1,2]pyrido[3,4-b]indol-4-iumchloride (8bkd), 2-(4-Fluorophenyl)-5-(methoxycarbonyl)-1-methyl-2,11-dihydroimidazo[1′,5′:1,2]pyrido[3,4-b]indol-4-ium chloride (8bpd), 8-Bromo-1-(2-bromophenyl)-2-phenyl-2,11-dihydroimidazo[1′,5′:1,2]pyrido[3,4-b]indol-4-ium chloride (8ccd), 1-(4-Bromophenyl)-2-(4-chlorophenyl)-11-methyl-2,11 dihydroimidazo[1′,5′:1,2]pyrido[3,4-b]indol-4-iumchloride (8dbc), 1-(4-Bromophenyl)-2-(4-fluorophenyl)-11-methyl-2,11-dihydroimidazo[1′,5′:1,2]pyrido[3,4-b]indol-4-iumchloride (8dbd), 1,2-Bis(4-chlorophenyl)-11-methyl-2,11-dihydroimidazo[1′,5′:1,2]pyrido[3,4-b]indol-4-iumchloride (8ddc), 2-Benzyl-1-(4-bromophenyl)-2,11-dihydroimidazo[1′,5′:1,2]pyrido[3,4-b]indol-4-ium chloride (9aaa), 1-(4-Bromophenyl)-2-(4-fluorobenzyl)-2,11-dihydroimidazo[1′,5′:1,2]pyrido[3,4-b]indol-4-ium chloride (9aab), 1-(4-Bromophenyl)-2-(4-(tert-butyl)benzyl)-2,11-dihydroimidazo[1′,5′:1,2]pyrido[3,4-b]indol-4-ium chloride (9aac), 1-(4-Bromophenyl)-2-(4-(trifluoromethyl)benzyl)-2,11-dihydroimidazo[1′,5′:1,2]pyrido[3,4-b]indol-4-ium chloride (9aad), 2-(4-Methoxybenzyl)-1-phenyl-2,11-dihydroimidazo[1′,5′:1,2]pyrido[3,4-b]indol-4-ium chloride (9aae), 1-(4-Bromophenyl)-2-(2,5-difluorophenyl)-2,11-dihydroimidazo[1′,5′:1,2]pyrido[3,4-b]indol-4-ium chloride (9aaf), 1-(4-Bromophenyl)-2-(3,4-dimethoxybenzyl)-2,11-dihydroimidazo[1′,5′:1,2]pyrido[3,4-b]indol-4-ium chloride (9aag), 1-(4-Chlorophenyl)-2-(4-methoxybenzyl)-2,11-dihydroimidazo[1′,5′:1,2]pyrido[3,4-b]indol-4-ium chloride (9abe), 1-(4-Chlorophenyl)-2-(4-(trifluoromethoxy)benzyl)-2,11-dihydroimidazo[1′,5′:1,2]pyrido[3,4-b]indol-4-ium chloride (9abh), 1-(4-Chlorophenyl)-2-(3,4-dimethoxybenzyl)-2,11-dihydroimidazo[1′,5′:1,2]pyrido[3,4-b]indol-4-ium chloride (9abg), 1-(4-Chlorophenyl)-2-(thiophen-3-ylmethyl)-2,11-dihydroimidazo[1′,5′:1,2]pyrido[3,4-b]indol-4-ium chloride (9acj), 2-(4-Fluorophenyl)-1-phenyl-2,13-dihydroimidazo[1,5-a]indolo[2,3-c]quinolin-4-ium chloride (12ad), 1-(4-Bromophenyl)-2-(4-chlorophenyl)-2,13-dihydroimidazo[1,5-a]indolo[2,3-c]quinolin-4-ium chloride (12bc), 1-(4-Bromophenyl)-2-(4-fluorophenyl)-2,13-dihydroimidazo[1,5-a]indolo[2,3-c]quinolin-4-ium chloride (12bd), 1,2-Bis(4-chlorophenyl)-2,13-dihydroimidazo[1,5-a]indolo[2,3-c]quinolin-4-ium chloride (12cc), 2-(4-Bromophenyl)-1-(4-chlorophenyl)-2,13-dihydroimidazo[1,5-a]indolo[2,3-c]quinolin-4-ium chloride (12db) and 2-(4-Fluorophenyl)-1-(p-tolyl)-2,13-dihydroimidazo[1,5-a]indolo[2,3-c]quinolin-4-ium chloride (12dd).

5. A process for preparing the compound of formula I, 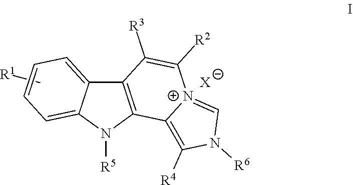
wherein:
R¹ is selected from hydrogen, halogen, alkoxy, benzyloxy, sulphonic acid, sulphonamide, cyano, nitro or amide;
R² is selected from hydrogen, C₁₋₂ alkyl, alkyloycarbonyl or aryloxycarbonyl;
R³ is selected from hydrogen or aryl;
R² and R³ may jointly form an aromatic ring having substituent selected from hydrogen, trifluoromethyl, nitro or halogen;
R⁴ is selected from substituted or unsubstituted alkyl, aryl or heteroaryl, wherein the substitutents are selected from the groups consisting of, halogen, alkyl, aryl, alkoxy, aryloxy, thiomethyl, trifluoromethyl, trifluoromethyloxy, thioalkyl, sulphonylamide or nitro;
R⁵ is selected from H, alkyl, alkenyl, aryl, arylalkyl, heteroaryl, benzoyl, sulphonylphenyl, or alkoxycarbonyl;
R⁶ is selected from substituted or unsubstituted alkyl, aryl, arylalkyl, 5 or 6-membered heteroaryl, alkoxycarbonylalkyl, cycloalkyl, or hetercycloalkyl, wherein the substitutents are selected from halogen, alkyl, alkoxy, aryl or alkyl aminoalkyl and
X is selected from Cl, I, ClO₄, CF₃SO₂, BF₄ or PF₆, comprising the steps of:
a) reacting an indole derivative of formula A with a compound of formula A′ selected from arylpyruvaldehyde, terminal alkyne or substituted acetophene in the presence of iodine in DMSO as solvent for 1 to 12 h at rt −110° C., washing the reaction mixture with Na₂S₂O₃ solution and extracting with an organic solvent to obtain a compound of formula B, 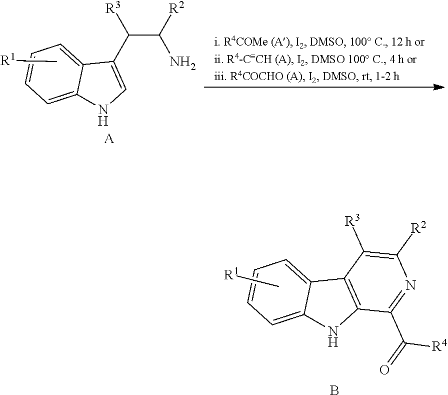
wherein R¹, R², R³ and R⁴ are as defined above;
b) evaporating the organic solvent and performing the column chromatography over silica gel using hexane: ethylacetate as eluent to purify the compound of formula B;
c) reacting the compound of formula B with R⁵X, wherein R⁵ is selected from H, alkyl, alkenyl, aryl, arylalkyl, heteroaryl, benzoyl, sulphonylphenyl, alkoxycarbonyl and X is a halogen, in the presence of cesium carbonate in DMF and adding crushed ice to obtain solid precipitate of the N-substituted carboline compound of formula C; 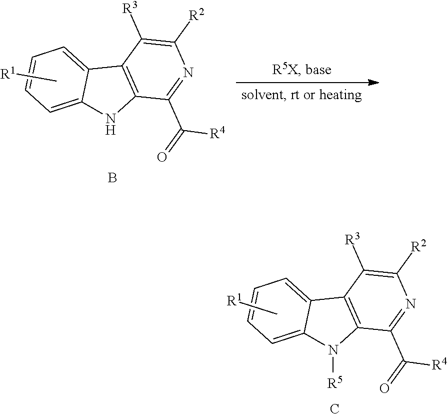
d) reacting formaldehyde with the substituted p-carboline compound of formula B or C and amine of formula R⁶NH₂, wherein R⁶ is selected from substituted or unsubstituted alkyl, aryl, arylalkyl, 5 or 6-membered heteroaryl, alkoxycarbonylalkyl, cycloalkyl, hetercycloalkyl, wherein the substitutents are selected from halogen, alkyl, alkoxy, aryl or alkyl aminoalkyl, to obtain compound for formula I. 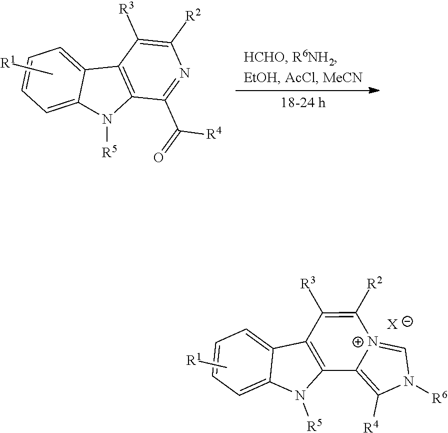
e) optionally purifying the compound for formula I by column chromatography over silica gel (MeOH/EtOAc).

6. The process as claimed in claim 5, wherein the indole derivative is selected from the group consisting of tryptamine, alkyl tryptophan ester and 2-(1H-indole-3-yl)aniline.

7. The process as claimed in claim 5, wherein the organic solvent is selected from ethyl acetate, dichloromethane or chloroform.

8. The process as claimed in claim 5, wherein in step (a) 1-formyl-9H-β-carboline is reacted with nitromethane and NH₄OAc at 80-90° C. for 15 min, extracted with ethylacetate and subjected to column chromatography over silica gel to obtain 1-acetyl-9H-β-carboline.

9. A pharmaceutical composition comprising of an effective amount of compound as for formula I as claimed in claim 1, or a pharmaceutical acceptable salt thereof and a pharmaceutically acceptable carrier.

10. The composition as claimed in claim 9, wherein the pharmaceutically acceptable carrier is suitable for systemic or oral administration.

11. A compound according to any one of claims 1-4, or a composition according to any one of claims 9-10, for use in a method for preventing, ameliorating or treating a disease, disorder or syndrome.

12. The compound or composition for use according to claim 11, wherein the disease, disorder or syndrome is neuropathic pain, visceral pain, drug addiction, hyperalgesia, arthritic inflammation, autoimmune inflammation or a combination thereof.

13. A method for preventing, ameliorating or treating a disease, disorder or syndrome in a subject in need thereof comprising administering to the subject a therapeutically effective amount of a compound according to any one of claims 1-4 or a composition according to any one of claims 9-10.

14. The method according to claim 13, wherein the disease, disorder or syndrome is neuropathic pain, visceral pain, drug addiction, hyperalgesia, arthritic inflammation, autoimmune inflammation, or a combination thereof.

15. Use of a compound according to any one of claims 1-4, or a composition according to any one of claims 9-10, in the manufacture of a medicament for the treatment of pain, substance abuse, and pruritus in a subject in need thereof, wherein the medicament is to be administered to the subject.
